(19) United States
(12) Patent Application Publication
(10) Pub. No.: US 2022/0381787 A1
(43) Pub. Date: Dec. 1, 2022

(54) NOVEL LOX-1 ANTIBODY COMPOSITIONS, LOX1 NEUTRALIZATION ASSAY AND METHODS OF TREATMENT USING SAME

(71) Applicant: The Wistar Institute of Anatomy and Biology, Philadelphia, PA (US)

(72) Inventors: Dmitry I. Gabrilovich, Villanova, PA (US); Kar Muthumani, Cherry Hill, NJ (US)

(21) Appl. No.: 17/634,968

(22) PCT Filed: Aug. 12, 2020
(86) PCT No.: PCT/US2020/045952

§ 371 Date: Feb. 12, 2022

Related U.S. Application Data
(60) Provisional application No. 62/886,185, filed on Aug. 13, 2019.

(51) Int. Cl. G01N 33/574; C07K 16/28

(52) U.S. Cl. CPC G01N 33/57492; C07K 16/28; C07K 2317/31; G01N 2333/705

(57) ABSTRACT

Provided herein is a recombinant antibody or an epitope binding fragment thereof that specifically binds to a lectin-like oxidized low-density lipoprotein (LDL) receptor-1 (LOX-1) epitope, as well as compositions and methods using these antibodies and fragments for therapeutic and diagnostic protocols.

### CROSS-REFERENCE TO RELATED APPLICATIONS

This application claims the benefit of the priority of U.S. Provisional Patent Application No. 62/886,185, filed Aug. 13, 2019, which application is incorporated herein by reference.

### STATEMENT REGARDING FEDERALLY SPONSORED RESEARCH OR DEVELOPMENT

This invention was made with government support under grant numbers R01CA165065 and P30CA010815 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “WST183PCT_ST25.txt”, created Aug. 12, 2020, and having 45 kilobytes (KB) in size.

### BACKGROUND OF THE INVENTION

Lectin-like oxidized low-density lipoprotein (LDL) receptor-1 (LOX-1) is an endothelial receptor for oxidized low-density lipoprotein (oxLDL), a key molecule in the pathogenesis of several diseases and may play a role as a scavenger receptor. LOX-1 is a transmembrane protein with 273 residues comprising 4 domains. The first 36 residues form a cytoplasmic tail, followed by a single transmembrane domain (21 residues), and an extracellular region comprising two domains. The first extracellular domain (aa58-142) is predicted to be a coil, and the second extracellular domain (aa143-273) is a C-type lectin-like domain (CTLD) responsible for ox-LDL recognition. It exists as a disulfide-linked homodimer.

LOX-1 is expressed on the surface of endothelial cells as well as several other cell types, including smooth muscle cells, platelets, and fibroblasts, it is linked to multiple functions including immune regulation, hypercholesteremia, hypertension, obesity and diabetes. LOX-1 is also expressed on the surface of other immune cells, such as dendritic cells (DCs) and macrophages. The expression level of LOX-1 can be modulated by inflammatory stimuli as well as by its ligands (Yoshimoto, R. et al. 2011 The Discovery of LOX-1, its Ligands and Clinical Significance, Cardiovascular Drug Target, 25:379-391, doi: 10.1007/s10557-011-6324-6), and targeting LOX-1 with a tumor antigen using anti-LOX-1 antibody 23C11 induces anti-tumor immunity. (Delneste et al Involvement of LOX-1 in dendritic cell-mediated antigen cross-presentation. Immunity. 2002 September; 17(3):353-62.

Further, LOX-1 expression is identified on a distinct population of neutrophils in cancer patients which are associated with accumulation of polymorphonuclear myeloid-derived suppressor cells (PMN-MDSC) and therefore serve as a marker of PMN-MDSC associated with ER stress and lipid metabolism in humans. See, for example, Condamine et al Lectin-type oxidized LDL receptor-1 distinguishes population of human polymorphonuclear myeloid-derived suppressor cells in cancer patients. Sci Immunol. 2016 August; 1(2): pii: aaf8943. doi: 10.1126/sciimmunol.aaf8943. Epub 2016 Aug. 5; Kim et al The Ratio of Peripheral Regulatory T Cells to Lox-1+ Polymorphonuclear Myeloid-derived Suppressor Cells Predicts the Early Response to Anti-PD-1 Therapy in Patients with Non-Small Cell Lung Cancer. Am J Respir Crit Care Med. 2019 Jan. 15; 199(2):243-246. doi: 10.1164/rccm.201808-1502LE.; and US Patent Application Publication Nos. US-2018-0059115 and US-2018-0164313.

PMN-MSC are important regulators of immune responses in cancer supports tumor progression. LOX-1 is practically undetectable in neutrophils in peripheral blood of healthy donors, whereas 5-15% of total neutrophils in cancer patients and 15-50% of neutrophils in tumor tissues are LOX-1 positive (LOX-1+). In contrast to their LOX-1 negative (LOX-1−) counterparts, LOX-1+ neutrophils exhibit a gene signature associated with immune suppressive activity and up-regulation of ER stress, among other biochemical characteristics of PMN-MDSC. Moreover, induction of ER stress in neutrophils from healthy donors up-regulates LOX-1 expression and converted these cells to suppressive PMN-MDSC.

Several antibodies recognizing LOX-1 have been developed. See, for example, www.hycultbiotech.com/hm2138, www.biolegend.com/en-us/products/pe-anti-human-lox-1-antibody-8592, as well as US Patent Application Publication Nos. US-2008-0267984 and US-2009-0203039. These antibodies recognize LOX-1 but have absent or undesired neutralizing activity.

### SUMMARY OF THE INVENTION

In one aspect, provided herein is a recombinant antibody or an epitope binding fragment thereof that specifically binds to a lectin-like oxidized low-density lipoprotein (LDL) receptor-1 (LOX-1) epitope. The antibody or fragment comprises at least one of: a heavy chain variable region encoded by a nucleic acid sequence of SEQ ID NOs: 2, 6, 10, 14, 18, or 24, or a nucleic acid sequence at least 85% identical thereto; or a light chain variable region encoded by a nucleic acid sequence of SEQ ID NOs: 4, 8, 12, 16, 20, 22, or 26, or a nucleic acid sequence at least 85% identical thereto; or a heavy chain variable region having an amino acid sequence of SEQ ID NOs: 3, 7, 11, 15, 19, or 25, or an amino acid sequence at least 85% identical thereto; or a light chain variable region having an amino acid sequence of SEQ ID NOs: 5, 9, 13, 17, 21, 23, or 27, or an amino acid sequence at least 85% identical thereto. In one embodiment, the antibody or epitope binding fragment thereof comprises one or more of variable regions of 3D8, 6A10, 9E12, 12D9, 12E4 and 4D6 antibodies. In one embodiment, the antibody or epitope binding fragment thereof recognizes an epitope of LOX-1 protein in native condition. In one embodiment, the recombinant or monoclonal antibodies 3D8, 6A10, 9E12, 12D9, 12E4 and 4D6 are provided. In certain embodiments, provided is a host cell producing the antibody or epitope binding fragment thereof. These antibodies are able to neutralize LOX-1 mediated signaling.

In another aspect, provided a pharmaceutical composition, a diagnostic composition, or a kit comprising one or more of the antibody or epitope binding fragment thereof.

In yet another aspect, a method is provided for monitoring the population of polymorphonuclear myeloid derived suppressor cells (PMN-MDSCs) in a subject comprising contacting a biological sample from the subject containing polymorphonuclear neutrophils (PMNs) and PMN-MDSC with one or more of the antibody or epitope binding fragment thereof; and detecting and optionally distinguishing/separating LOX-1 positive (LOX-1+) cells/neutrophils/PMNs from LOX-1 negative (LOX-1−) cells/neutrophils/PMNs in the sample, wherein the LOX-1+ cells/neutrophils/PMNs are PMN-MDSCs substantially free of PMN, in one embodiment, the method may be used for diagnosing a cancer, a cancer progression or metastasis in a subject. In one embodiment, the subject is diagnosed with a cancer if percentage of LOX-1⁺ cells in the total neutrophils in the sample is greater than a control.

In a further aspect, provided is a method for evaluating differentiation of PMN-MDSCs from polymorphonuclear neutrophils (PMNs) in a biological sample containing both types of cells, or a method of identifying an antagonist or inhibitor of LOX-1 expression, or for diagnosing a cancer, or monitoring a cancer progression or metastasis in a subject. Such method utilized one or more of the antibody or epitope binding fragment thereof and/or the composition as described in detecting LOX-1 positive cells/neutrophils/PMNs/PMN-MDSCs.

Also provided is a method of treating a cancer (for example, a solid cancer) in a subject comprising administering an effective amount of a composition that reduces or inhibits ER stress response in mammalian neutrophils or reduces or inhibits LOX-1 expression on neutrophil populations (for example, an ER stress antagonist, B-I09), and/or administering an effective amount of one or more of the antibody or epitope binding fragment thereof as described herein, or a LOX-1 antagonist or inhibitor identified using the method described herein.

Other aspects and advantages of these compositions and methods are described further in the following detailed description of the embodiments thereof.

### BRIEF DESCRIPTION OF THE DRAWINGS

FIG. 1 is a graph showing results of human LOX-1 immunization and antibody responses in mouse immune sera, 100% seroconversion is observed after immunization series using human LOX-1 (hLOX-1) antigen. Mice were immunized and seroconversion of mice sera was evaluated as indicated as in Example.

FIG. 2 is a bar graph showing sensitivity and specificity of anti-hLOX-1 monoclonal antibodies (mAbs) for recombinant protein. Detection of anti-hLOX-1 mAbs was performed by ELISA against recombinant hLOX-1 as capture. The specific anti-hLOX-1 mAb binder is listed by clone number.

FIG. 3 shows a series of 12 graphs characterizing mAbs IgGs avidity to hLOX-1 recombinant protein. Anti-hLOX-1 mAbs samples were tested for anti-hLOX-1-specific binding to recombinant antigen using a modified ELISA and avidity determined by 4M urea washes against recombinant human LOX-1 protein.

FIG. 4 shows 6 graphs characterizing mAbs IgGs avidity to hLOX-1 recombinant protein. Anti-LOX-1 mAbs samples were tested for anti-LOX-1-specific binding to recombinant antigen using a modified ELISA and avidity determined by 4M urea washes against recombinant human LOX-1 protein. Most clones showed low avidity. Overall out of 16 clones identified, 6 were stronger binders, and 4 were high binders in rank order of strength of binding 6A 10=12D9>12E4>4D6.

FIGS. 5A and 5B provide a series of gels showing the binding and specificity of anti-LOX-1 mAbs to target protein analyzed by Western blot analysis. In each figure, the first row of gels was loaded with denatured LOX-1, while the second row of gels was loaded with native LOX-1, as indicated in Example. The clone numbers of the anti-LOX-1 antibodies are listed below the gels.

FIG. 6 shows three graphs characterizing the measure of the overall strength of an antibody-antigen complex of three anti-hLox1 mAb clones, 4D6, 12D9 and 6A10 measured by the antibody Avidity assay.

FIGS. 7A-7C show the construction and expression of recombinant, anti-LOX-1 antibody clone 6A10. FIG. 7A Schematic representation of antibody cloning. The anti-Lox1 (Clone 6A10) sequence was incorporated into IgG1 and IgG3 framework, sub-cloned into the protein expression vector pcDNA3.4 and recombinant antibodies were expressed in Expi293F cells. FIGS. 7B-7C are SDS-PAGE analyses of recombinantly expressed anti-Lox1 IgG1 and anti-Lox1-IgG3 antibodies under non-reducing and reducing conditions. Visualized by Coomassie blue staining.

FIGS. 8A-8B show that recombinantly expressed monoclonal antibodies are immunoreactive and functional. FIG. 8A is a flow cytometric histogram showing fluorescent intensity of cells incubated with recombinantly expressed anti-LOX-1 (IgG1) on the plasmid hLOX-1 transfected cells. Anti-hLOX-1/DNA transfected cells were stained with 1 μg/ml of anti-Lox1 (IgG1) (6A10) antibody plus FITC-labelled goat anti-human IgG secondary antibody. Specific binding to cells transfected with LOX-1 antigen present on the cell surface of 293T cells (filled) and no binding above background to non-transfected cells (open). FIG. 8B show flow cytometry analysis of PBMCs from whole blood. Flow cytometry analysis of anti-hLOX-1 binding to CD11c⁺, CD14⁺, CD19⁺ and CD3⁺ cells in PBMCs from healthy donors. Flow cytometric analyses of anti-CD3, CD4, CD14, CD19 and anti-Lox1 monoclonal antibodies were used to detect their lineage specificity. Antibody binds to immune subset cells but not to CD3⁺ cells in PBMCs.

FIG. 9 shows an immunohistochemical analysis of LOX-1 expression in tumor tissues. 5 μm sized paraffin embedded tissue sections were de-paraffinized. Standard IHC protocol was followed to stain the tumor tissue samples using the monoclonal antibody against LOX-1 (IgG1) (1:10) for the detection of LOX-1 followed by staining with hematoxylin-eosin. LOX-1 detection was performed using a biotinylated anti-rabbit IgG secondary antibody and streptavidin-Horseradish peroxidase (HRP), followed by colorimetric detection. The LOX-1 antigen is stained brown. LOX-1 expression was observed, in various tumor tissues (liver, breast, bladder pancreatic, ovarian, kidney, colon, lung; see arrows).

FIGS. 10A and 10B show FACS analyses of PMN cells showing the expression of LOX-1. Representative histograms of untreated or Thapsigargin (THG)-treated cells were stained after incubating with anti-LOX-1 antibody (Ab) are shown. Percentage of LOX-1⁺ PMN and expression of LOX-1 in PMN isolated from a healthy donor and treated with 1 μM 1HG and 1 mM Dithiothreitol (DTT). Representative LOX-1 expression level with anti-LOX-1 and commercial Abs. Representative LOX-1 expression level with DMAb derived Abs. Summary of data derived from FACS analysis of LOX-1 level is expressed as the % of LOX-1⁺/CD15⁺. Higher percentages of PMN expressed LOX-1 after they were cultured from THG-treated cells.

FIGS. 11A-11B. Representative flow cytometric analysis of CD15⁺/LOX-1⁺ PMNCs from a pancreatic cancer patient sample. FIG. 11A shows the gating strategy for CD15⁺ and LOX-1⁺ in multi-parameter FACS which was conducted to examine LOX-1 surface expression in blood pancreatic cancer patient sample. FIG. 11B shows the percentage of CD15⁺/LOX-1⁺ in the patient sample. Anti-LOX-1 antibody binds to PMN from pancreatic cancer.

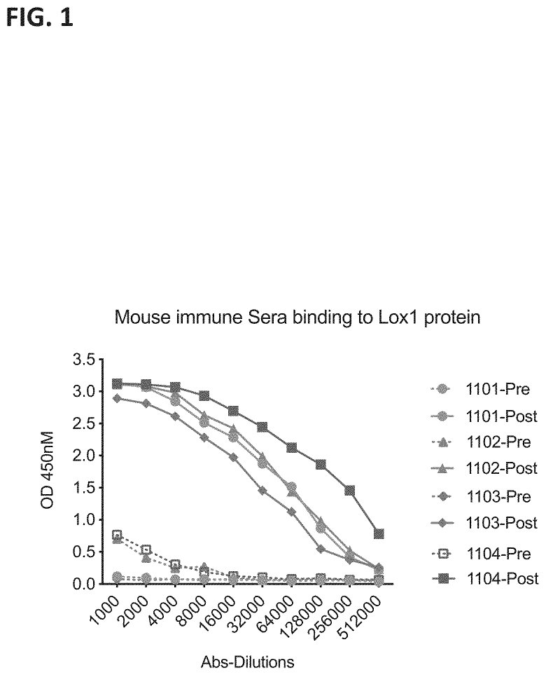

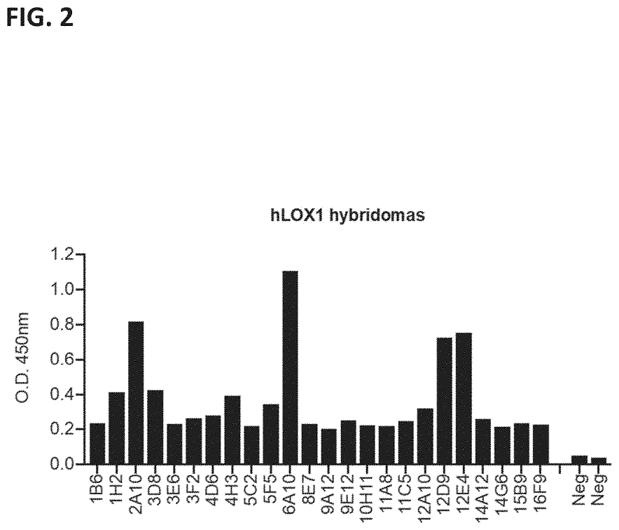

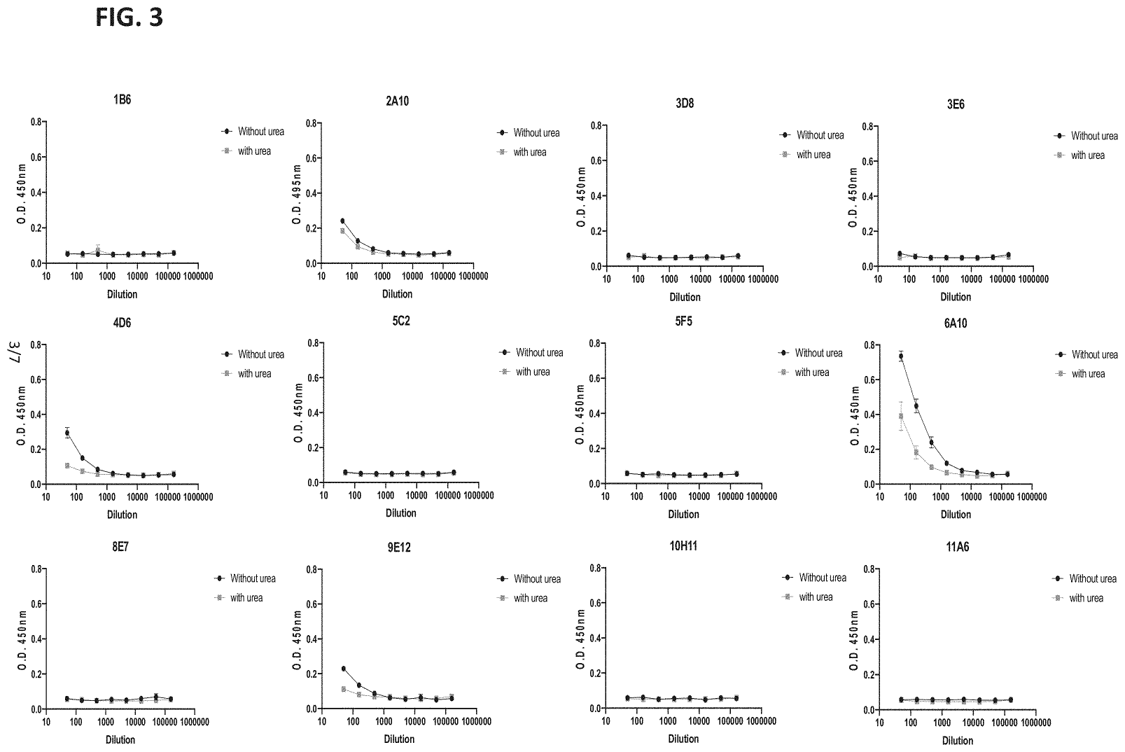

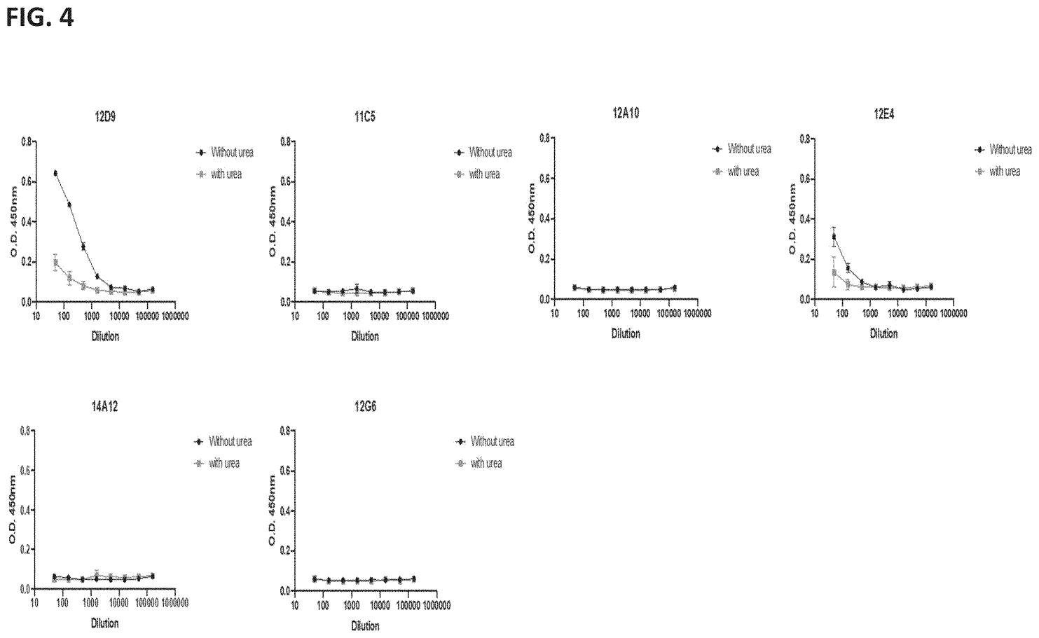

### DETAILED DESCRIPTION OF THE INVENTION

Antibodies and epitope binding fragments thereof recognizing a lectin-like oxidized low-density lipoprotein (LDL) receptor-1 (LOX-1) epitope are disclosed. In certain embodiments, the antibody or an epitope binding fragment thereof recognizes LOX-1 in its native condition or conformation, for example, LOX-1 expressed on cells (e.g., neutrophils, or PMN-MDSC) with strong avidity. In certain embodiments, the antibody or an epitope binding fragment thereof binds to LOX-1 in its native condition with stronger avidity compared to binding to LOX-1 in denatured condition.

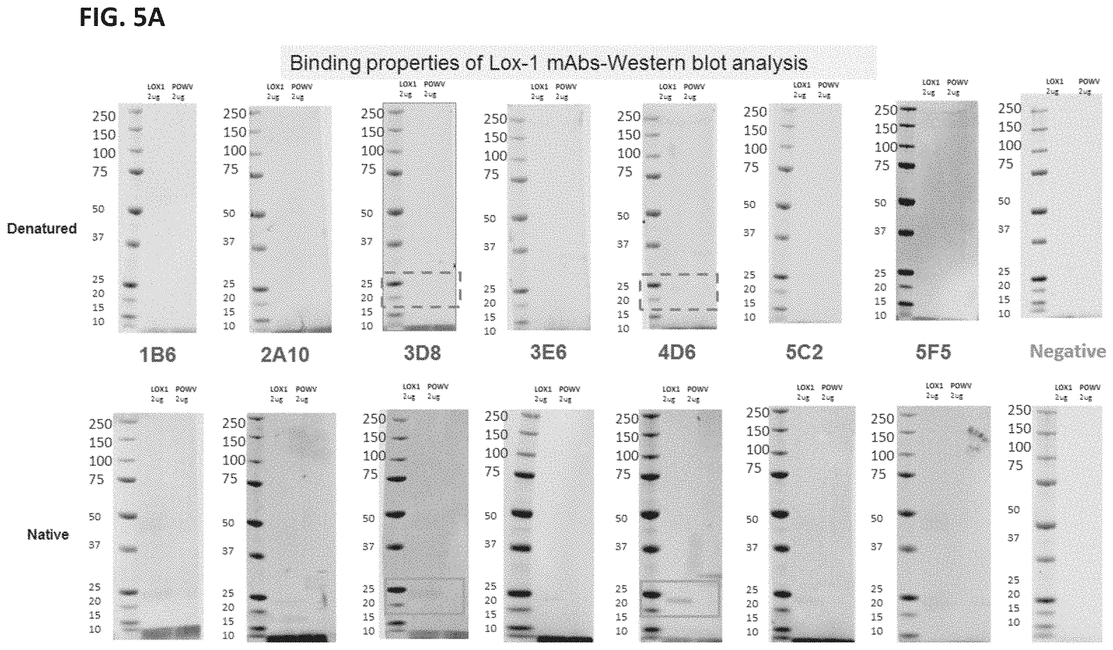

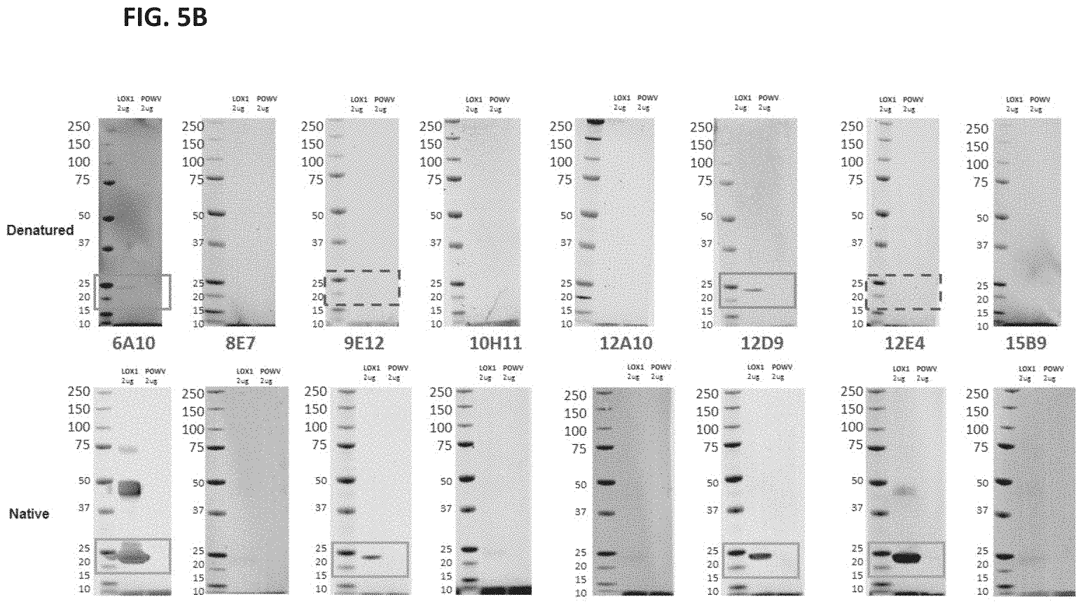

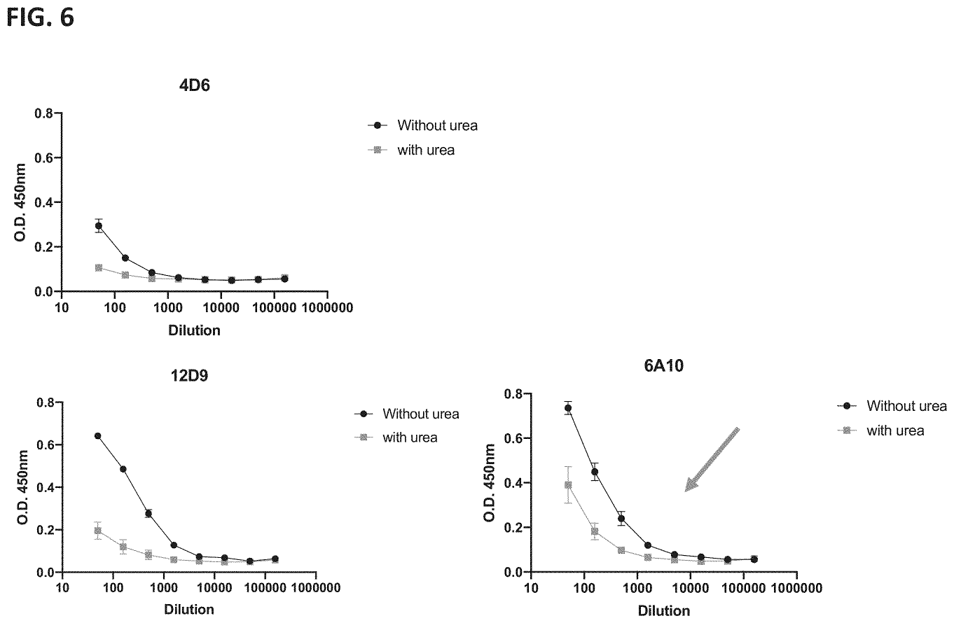

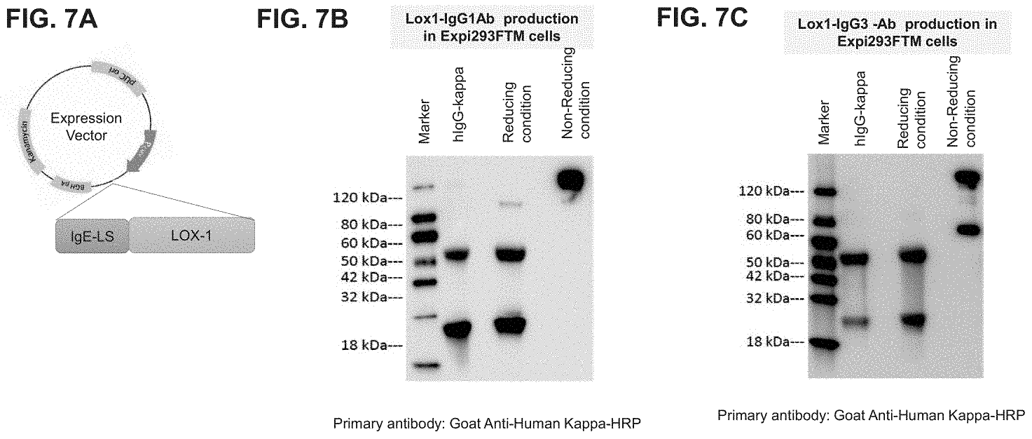

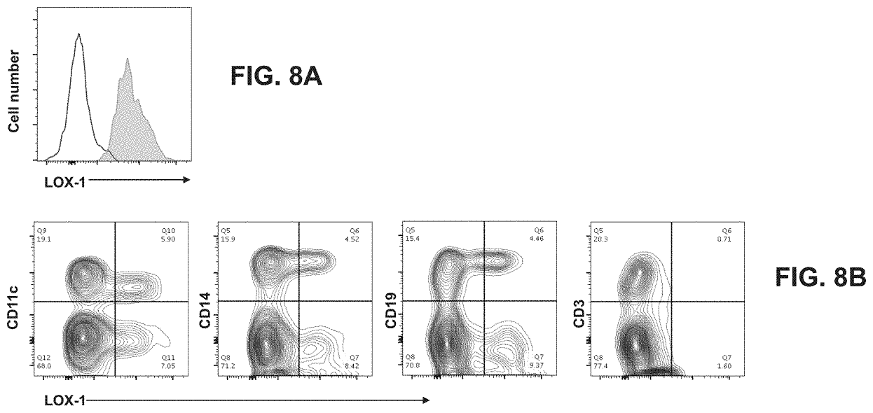

An immunization strategy was developed to generate monoclonal antibodies (mAb) against human LOX-1 protein, comprising administering a human LOX-1 coding DNA sequence to a subject and boosting the immunization via further administering a recombinant LOX-1 protein. Without wishing to be bound by the theory, such immunization strategy allows generation of antibodies recognizing conformation sensitive epitope(s) and enables detection of LOX-1 in its native condition. For example, the generated antibodies or epitope binding fragments thereof may be used to identify a cell expressing LOX-1 (on the cell surface or in the cytosol) optionally by fluorescence-activated cell sorting (FACS). Such uses are supported by the Examples as well as by FIGS. 5A and 5B, showing that anti-LOX-1 antibody clones 3D8, 4D6, 6A10, 9E12, 12D9 and 12E4 demonstrate binding to native LOX-1 but not denatured LOX-1 (or binding to denatured LOX-1 at a significantly lower level).

Also disclosed are methods and compositions useful in detecting LOX-1 protein, or in detecting, identifying, separating or isolating cells expressing LOX-1 (on the cell surface or in the cytosol), or in detecting, identifying, separating or isolating cells indicative of cancer in a mammalian subject (for example, polymorphonuclear myeloid derived suppressor cells (PMN-MDSCs)), using one or more of the antibodies or epitope binding fragments thereof as described herein. Additionally, diagnostic compositions and methods are disclosed herein that facilitate identifying an inhibitor of LOX-1 expression and/or PMN-MDSC differentiation, and/or diagnosing a cancer, cancer progression or metastasis.

Further, therapeutic composition and methods are provided for use in treating a cancer via reducing, neutralizing, and/or deleting PMN-MDSCs using one or more of the antibodies or epitope binding fragments thereof or composition as described herein. Optionally, a subject in need thereof may also receive the therapeutic composition in combination with other therapy, for example, B-I09, an ER-stress antagonist, as described in detail below.

### Components of the Methods

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

In one embodiment, an “antibody” refers to an intact immunoglobulin, such as an IgG, or to an antigen binding portion thereof that competes with the intact antibody for specific binding, or a modification thereof, unless otherwise specified. In certain embodiments, an intact antibody is an IgA, IgG, IgM, IgE or IgD. In one embodiment, an intact antibody is an IgG1. IgG2, IgG3 or IgG4. An antibody (e.g., an antibody, an antibody heavy chain, an antibody light chain, or any fragment or modification thereof) comprises three Complementarity-Determining Regions (CDRs, also known as HV, hypervariable regions, namely CDR1, CDR2, CDR3, from N-terminal to C-terminal, or 5′ to 3′ when corresponding nucleic acid sequence is referred to), and four framework regions (FRs, namely FR1, FR2, FR3 and FR4, from N-terminal to C-terminal, or 5′ to 3′ when corresponding nucleic acid sequence is referred to). See, e.g., Janeway, Charles A Jr; Travers, Paul; Walport, Mark; Shlomchik, Mark J (2001). Immunobiology: The immune System in Health and Disease (5 ed.). New York: Garland Science. ISBN 0-8153-3642-X, which is incorporated herein by its entirety. In the antibody construct, CDRs are arranged non-consecutively, not immediately adjacent to each other, and may be separated by an FR. For example, an antibody may be organized as variable domains/regions (such as FR1-CDR1-FR2-CDR2-FR3-CDR3-FR4) following by optional constant regions. As part of the variable chain/region/domain in an antibody construct and T cell receptors generated by B-cells and T-cells respectively, CDRs refer to the region where an antigen/epitope specifically binds. The terms “variable region” and “variable domain” are used interchangeably and refer to the portion of an antibody having an amino acid sequence that determines the antigenic specificity of the antibody.

The antibody or fragment includes a monoclonal antibody, such as the 3D8, 6A10, 9E12, 12D9, 12E4 and 4D6 antibodies which were the sources of the heavy chain and light chain variable region sequences SEQ ID NOs: 3, 5, 7, 9, 11, 13, 15, 17, 19, 21, 23, 25 and 27 and the heavy chain and light chain variable region coding sequences SEQ ID NO: 2, 4, 6, 8, 10, 12, 14, 16, 18, 20, 22, 24 and 26. Such antibodies can also include a synthetic antibody, a recombinant antibody, a chimeric antibody, a humanized antibody, a human antibody, a CDR-grafted antibody, a multi-specific binding construct that can bind two or more epitopes, a dual specific antibody, a bi-specific antibody, an affinity matured antibody, a single antibody chain or an say fragment, a diabody, a single chain comprising complementary scFvs (tandem scFvs) or bispecific tandem scFvs, an Fv construct, a disulfide-linked Fv, a Fab construct, a Fab′ construct, a F(ab′)₂ construct, an Fe construct, a monovalent or bivalent construct from which domains non-essential to monoclonal antibody function have been removed, a single-chain molecule containing one V_{L} (variable region of light chain), one V_{H} (variable region of heavy chain) antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, or any recombinant versions thereof. Definitions and examples of these types of structures are found in the art and in, e.g., U.S. Pat. No. 9,902,772, incorporated by reference herein. In certain embodiments, an antibody also refers to an “antibody mimic” or an “antibody equivalent”.

As used herein, an “antibody mimic” or an “antibody equivalent” refers to affibodies, i.e., a class of engineered affinity proteins, generally small (˜6.5 kDa) single domain proteins that can be isolated for high affinity and specificity to any given target, aptamers, polypeptide molecules that bind to a specific target, an affilin, an affitin, an affimer, an alphabody, an anticalin, an avimer, a DARPin (designed ankyrin repeat proteins), a Fynomer, a Kunitz domain peptide, a monobody, a peptabody and others known in the art.

In one embodiment, an antibody is a minibody which is composed of a single-chain molecule containing one V_{L}, one V_{H} antigen-binding domain, and one or two constant “effector” domains. These elements are connected by linker domains. In still another embodiment, the antibodies useful in the methods and compositions herein are “unibodies”, which are IgG4 molecules from with the hinge region has been removed. The term “recombinant antibody” refers to an antibody that is generated by cloning the immune-specific heavy and light antibody coding sequences into a vector. In one embodiment, vector is designed for high-yield mammalian expression. The resulting vectors are introduced into expression hosts (e.g., bacteria, virus, yeast or mammalian) for the manufacturing of high-quality functional antibodies. Generally, the coding sequence is not naturally associated with the host cell. Recombinant antibodies have glycosylation patterns that differ from the glycosylation pattern of an antibody having the same sequence if it were to exist in nature. In one embodiment, a recombinant antibody is expressed in a mammalian host cell which is not a human host cell. Notably, individual mammalian host cells have unique glycosylation patterns. Recombinant antibodies can be constructed in vitro by forming an Ig-framework through cloning of scFV or Fab or can be produced from an existing hybridoma. In hybridoma-based recombinant antibody generation, mouse, rat, and rabbit models are commonly used. However, as long as the appropriate oligonucleotide primers are available, recombinant antibodies can be developed from any species.

Anti-LOX-1 antibodies, LOX-1 antagonists or inhibitors, activators or regulators of ER stress response or other targeted biomarkers, and ligands that specifically bind to or form a complex with a neutrophil, a polymorphonuclear neutrophil (PMN), or a polymorphonuclear myeloid derived suppressor cell (PMN-MDSC) or a biomarker thereof may also be any of these forms of antibody or fragments.

Unless otherwise indicated, the term “anti-LOX-1 antibody” as used herein, includes, the monoclonal IgG immunoglobulins 3D8, 6A10, 9E12, 12D9, 12E4 and 4D6, comprising two full-length heavy chains (each chain comprising a variable region and a constant region) and two full-length light chains (each chain comprising a variable region and a constant region), as well as modifications, antigen/epitope binding fragments, as well as “antibody mimics” or “antibody equivalents” or constructs of fragments having one or more of the CDRs as described herein, and/or having one or more of the heavy chain and light chain variable region sequences SEQ ID NO: 3, 5, 7, 9, 11, 13, 15, 17, 19, 21, 23, 25 and 27 and/or encoded by one or more of the CDR coding sequences as described herein, and/or encoded by one or more of the heavy chain and light chain variable region coding sequences SEQ ID NO: 2, 4, 6, 8, 10, 12, 14, 16, 18, 20, 22, 24 and 26. In one embodiment, the antibody or epitope binding fragments as described herein refers to an anti-LOX-1 antibody or fragment encoded by a nucleic acid sequence of one of SEQ ID NO: 2, 4, 6, 8, 10, 12, 14, 16, 18, 20, 22, 24 and 26 or a nucleic acid sequence at least 85% identical thereto. Additionally or alternatively, the antibody or epitope binding fragments as described herein comprises an amino acid sequence of one of SEQ ID NO: 3, 5, 7, 9, 11, 13, 15, 17, 19, 21, 23, 25 and 27 or an amino acid sequence at least 85% identical thereto.

As used herein, by “at least 85% identical” encompasses at least 86%, 87%, 88%, 89%, 90%, 91%, 92%, 93%, 94%, 95%, 96%, 97%, 98%, 99%, up to 100% sequence identity with the identified reference sequence.

As used herein, an antagonist or inhibitor for LOX-1 may also refer to a radioactive particle, a chemical compound, a biological molecule (e.g., antibody), or any combination thereof, such as a biological molecule covalently or non-covalently attached to or associated with a chemical compound or a radioactive particle, which is able to reduce expression of LOX-1 protein in a cell or on a cell surface, or reduce LOX-1 positive cells in a cell population.

The endoplasmic reticulum (ER) is a multifunctional organelle required for lipid biosynthesis, calcium storage, and protein folding and processing. A number of physiological and pathological conditions, as well as a variety of pharmacological agents, are able to disturb proper ER function and thereby cause ER stress, which severely impairs protein folding and therefore poses the risk of proteotoxicity. Specific triggers for ER stress include, for example, particular intracellular alterations (e.g., calcium or redox imbalances), certain microenvironmental conditions (e.g., hypoglycemia, hypoxia, and acidosis), high-fat and high-sugar diet, a variety of natural compounds thapsigargin, tunicamycin, and geldanamycin), and several prescription drugs (e.g., bortezomib/Velcade, celecoxib/Celebrex, and nelfinavir/Viracept). The cell reacts to ER stress by initiating a defensive process, called the unfolded protein response (UPR), which is comprised of cellular mechanisms aimed at adaptation and safeguarding cellular survival or, in cases of excessively severe stress, at initiation of apoptosis and elimination of the faulty cell. The ER stress response includes three major signaling cascades initiated by three protein sensors: PERK (protein kinase RNA (PKR)-like ER kinase), IRE-1 (inositol-requiring enzyme 1) and ATF6 (activating transcription factor 6). Other regulators or activator or ER stress can be found in, for example, US Patent Application Publication No. US-2018-0059115.

B-I09 is an ER stress antagonist, which may be utilized in certain embodiments of the composition and methods. It is cell permeable and inhibits IRE1 endonuclease. Information about B-I09 is publicly available and can be found, for example, www.medchemexpress.com/B_I09.html, and www.tocris.com/products/b-i09_6009.

As used herein, a “modification” of an antibody or a grammatical variation thereof refers to an antibody in which one or more amino acid residues are inserted into, deleted from, and/or substituted into its amino acid sequence(s), e.g., any of amino acid sequence encoding the variable light or heavy chains, and/or CDRs of antibodies 3D8, 6A10, 9E12, 12D9, 12E4 and 4D6. One such modification is the replacement of one amino acid in such a sequence, e.g., any of amino acid sequences encoded by SEQ ID NOs: 2, 4, 6, 8, 10, 12, 14, 16, 18, 20, 22, 24 and 26 or amino acid sequences of 3, 5, 7, 9, 11, 13, 15, 17, 19, 21, 23, 25 and 27 with a conservative amino acid. In certain embodiments, the modification is in a FR region but not the CDR regions. Other modifications include, for example, fusion proteins formed by fusing the heavy chain of a selected antibody into an Ig backbone. Still another modification includes an anti-LOX-1 antibody that has been modified via conjugation to another chemical moiety (such as, for example, polyethylene glycol or albumin, e.g., human serum albumin), phosphorylation, and glycosylation. In another embodiment, a modification of any of antibodies 3D8, 6A10, 9E12, 12D9, 12E4 and 4D6 is a single chain human antibody, having a variable domain region from a heavy chain and a variable domain region from a light chain and a peptide linker connecting the heavy chain and light chain variable domain regions. In certain embodiments, an antibody as used herein also includes a modification of the antibody.

Other modifications include V(D)J recombination/rearrangement, e.g., a process by which T cells and B cells randomly assemble different gene segments—known as variable (V), diversity (D) and joining (J) genes (or regions, or segments, as used herein)—in order to generate unique receptors (known as antigen receptors) that can collectively recognize many different types of antigens. Briefly, the germ line (unrearranged) genomic DNA configuration of the immunoglobulin heavy chain locus comprises the tandem arrays of V, D, and J gene segments. A germ line kappa or lambda light chain locus comprises unreal-ranged V and J segments. Stepwise rearrangement of the germ line DNA results in the joining of a heavy chain D and J gene segment, followed by joining of a V segment to the D-J product, to generate the DNA encoding the heavy chain variable region. In the process of rearrangement, the ends of the gene segments are subject to variable amounts of exonuclease digestion and randomized non-templated bases are added at the segment ends, to produce additional sequence diversity at the VDJ junctional region that encodes the complementarity-determining region 3 (CDR3), which is often the region of the antibody heavy chain that has the greatest impact on antigen specificity. A similar process of V and J gene rearrangement with diversification of the VJ junction occurs in the light chain locus, to produce the rearranged light chain gene. See, e.g., Boyd et al, High-Throughput DNA Sequencing Analysis of Antibody Repertoires Microbiology Spectrum. 2.10.1128/microbiolspec.AID-0017-2014, which is incorporated herein by its entirety. Methods for producing such antibodies and antibody fragments are well-known in the art. Indeed, commercial vectors for certain antibody and antibody fragment constructs are available. The antibody may also be a protein (e.g., a fusion protein) comprising at least one antibody or antibody fragment, in a particular embodiment, the antibody comprises an Fe region.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein, the latter of which may be referred to as a conformation sensitive epitope or an epitope in native condition/conformation. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody or an epitope binding fragment thereof.

The term “LOX-1 epitope” as used herein refers to a portion/fragment of a LOX-1 protein or any naturally occurring or synthetic or recombinant amino acid sequence thereof that is capable of specifically complexing with an antibody, or epitope binding fragments or modified antibodies. In certain embodiments, the LOX-1 epitope is a conformation sensitive epitope. In certain embodiments, the LOX-1 epitope is a portion of LOX-1 protein which is only accessible for binding to an antibody or epitope binding fragment thereof when the LOX-1 protein is in its native conformation/condition, such as when expressed on/in a cell.

The term “LOX-1” as used herein is a cell surface endothelial receptor for oxidized low-density lipoprotein (lectin-like) receptor 1, first identified in endothelial cells as one of the main receptors for oxidized-LDL (ox-LDL). Ox-LDL is a key molecule in the pathogenesis of several diseases and may play a role as a scavenger receptor. Besides ox-LDL, the LOX-1 receptor has been shown to bind many different ligands including other modified lipoproteins, advanced glycosylation end products, aged red blood cells, apoptotic cells and activated platelets. LOX-1 has been involved in many different pathological conditions including atherogenesis, myocardial ischemia, hypertension, vascular diseases and thrombosis. Expression of LOX-1 can be induced by a wide array of stimuli including pro-inflammatory factor (TNF-α, IL-1β or IFN-γ), angiotensin II, endothelin-1, modified lipoproteins and free radicals. Engagement of LOX-1 can lead to induction of oxidative stress, apoptosis, endothelial dysfunction, fibrosis and inflammation through the activation of the NF-κB pathway. LOX-1 has also been described to play a role in tumorigenesis. Indeed, LOX-1 up-regulation has been observed during cellular transformation into cancer cell and can have a pro-oncogenic effect by activating the NF-κB pathway, by increasing DNA damage through increase ROS production and by promoting angiogenesis and cell dissemination. LOX-1 expression is identified on a distinct population of neutrophils in cancer patients which are associated with accumulation of PMN-MDSC and therefore serve as a marker of PMN-MDSC in humans. LOX-1 expressing neutrophils exhibit a gene signature associated with immune suppressive activity and up-regulation of endoplasmic reticulum (ER) stress, among other biochemical characteristics of PMN-MDSC. Additionally, induction of ER stress in neutrophils from healthy donors upregulates LOX-1 expression and converts these cells to suppressive PMN-MDSC. See, for example, US Patent Application Publication No. US20180059115.

“LOX-1”, “Lox-1”, “LOX1”, and “LOX 1” are used interchangeably herein. In certain embodiments, the LOX-1 protein sequence is found at Hugo Gene Nomenclature Committee 8133, Protein Sequence HPRD:04003. See, also, www.uniprot.org/uniprot/P78380 for more information regarding human LOX-1 (hLOX-1). In one embodiment, LOX-1 is a transmembrane protein with 273 residues comprising 4 domains and has a MW-25.5 kDa. It should be understood that the term LOX-1 can also represent the receptor protein in various species, and with conservative changes in the amino acid or encoding sequences, or with other naturally occurring modifications that may vary among species and between members of the same species, as well as naturally occurring mutations thereof. The nucleic acid sequence for the gene encoding LOX-1 (gene name OLR1) can be found in databases such as NCBI, i.e., NCBI gene ID: 4973 or Gene sequence: Ensembl:ENSG00000173391.

As used herein, the term “specific” or any grammatical variation thereof refers to antibodies, that bind to one or more epitopes of a protein or compound of interest, but which do not substantially recognize and bind other molecules in a sample containing a mixed population of antigenic biological molecules.

An antibody, fragment or modification thereof described herein may have a binding affinity and/or immunological specificity and/or avidity to its epitope at about 20%, about 40%, about 50%, about 60%, about 70%, about 80%, about 90%, about 95%, about 97%, about 99%, about 100%, more than about 100%, about 200%, about 300%, or about 500% of that of any known anti LOX-1 antibody. For example, a LOX-1 antibody is available from commercial sources, such as Biolegend Inc., San Diego, Calif. Conventional methods, including enzyme-linked immunosorbent assay (ELISA), radioimmunoassay (RIA), MSD assay, and antibody phage display library, may be used to determine such binding affinity and/or specificity. In certain embodiments, these anti-LOX-1 antibodies and fragments have a binding affinity (Ka) for a LOX-1 epitope that is sufficient to mediate binding on cultured cells and receptor internalization. In one embodiment, such Ka is between 0.1 to 1.5 M.

The term “isolate” or “separate” or “distinguish” or any grammatical variations thereof designates a biological material (nucleic acid or protein or cells) that has been removed from its original environment (the environment in which it is naturally present). For example, a PMN-MDSC present in its natural state in a biological sample is not isolated, however the same PMN-MDSC separated from other cells, such as LOX-1 negative PMNs/neutrophils in which it is naturally present, is considered “isolated” or “separated” or “distinguished”. The term “purified” does not require the material to be present in a form exhibiting absolute purity, exclusive of the presence of other compounds.

The terms “percent (%) identity”, “sequence identity”, “percent sequence identity”, or “percent identical” in the context of amino acid sequences or nucleotide sequences refers to the residues in the two sequences which are the same when aligned for correspondence. Percent identity may be readily determined for amino acid sequences or nucleotide sequences over the full-length of a protein, polypeptide, or encoding region thereof, e.g., about 15 amino acids, about 150 amino acids, or a peptide fragment thereof or the corresponding nucleic acid sequence coding sequences. A suitable amino acid fragment may be at least about 4 amino acids in length and may be up to about 200 or up to about 700 amino acids or nucleotide fragments of from about 12 nucleotides to about 600 to 2100 nucleotides. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., Thompson, J D et al, A comprehensive comparison of multiple sequence alignment programs. Nucl. Acids Res., 1999 July, 27(13):2682-90.

In certain embodiments, an “engineered” sequence refers to a coding sequence using degenerative codons changing the codon without affecting the amino acid sequence) in order to enhance expression of the coding sequence in any host, or in a subject (for example, human or mice), and/or in a tissue (for example, liver or muscle). Such optimization takes into consideration a variety of factors involved in different stages of protein expression, such as codon adaptability, and mRNA structure. This optimization may be performed using methods which are available on-line, published methods, or via a company which provides codon optimizing services. One codon optimizing method is described, e.g., in US Patent Application Publication No. US20160083748A1, which is incorporated by reference herein. Briefly, the nucleic acid sequence encoding the product is modified with synonymous codon sequences. By using one of these methods, one can apply the frequencies to any given polypeptide and produce a nucleic acid fragment of a codon-optimized coding region which encodes the polypeptide.

In certain embodiments, “indicating” or “correlating” or “comparing to a standard curve” as used herein, may be by any linear or non-linear method of quantifying the relationship between the level or amount of the antibody or epitope fragment thereof detected by binding to an LOX-1 epitope optionally on a cell surface, in comparison to a standard, control or comparative value for diagnosis or detection.

The terms “measuring” “determining” “detecting” are used interchangeably throughout and refer to detecting binding of a LOX-1 epitope (optionally on a cell surface or in a cell) to an antibody or epi tope binding fragment thereof. Measuring can be accomplished by methods known in the art and those further described herein.

A “suitable control,” “appropriate control” or a “control sample” is any control or standard familiar to one of ordinary skill in the art useful for comparison purposes. In one embodiment, a “suitable control” is a value, level, feature, characteristic, property (e.g., that of the presence or amount of PMN-MDSCs), that is determined in a healthy subject or a patient free of cancer or a patient free of cancer progression or a patient free of cancer metastasis. Other suitable controls include the same levels, etc., in a patient before anti-cancer treatment or after an anti-cancer treatment, or a patient without cancer metastasis. In certain embodiments, a control is a positive control which is a value, level, feature, characteristic, property of a patient with cancer/tumor, a certain type of cancer/tumor, or a cancer/tumor at certain stage during progression.

“Patient” or “subject” as used herein refer to a mammalian animal, including a human, a veterinary or farm animal, a domestic animal or pet, and animals normally used for clinical research. In one embodiment, the subject of these methods and compositions is a human.

The term “cancer” or “tumor” as used herein refers to, without limitation, refers to or describes the physiological condition in mammals that is typically characterized by unregulated cell growth. By cancer as used herein is meant any form of cancer, including hematological cancers, e.g., leukemia, lymphoma, myeloma, bone marrow cancer, and epithelial cancers, including, without limitation, breast cancer, lung cancer, small cell lung cancer, non-small cell lung cancer, prostate cancer, colorectal cancer, brain cancer, endometrial cancer, esophageal cancer, stomach cancer, bladder cancer, kidney cancer, pancreatic cancer, cervical cancer, head and neck cancer, ovarian cancer, colon cancer, melanoma, leukemia, myeloma, lymphoma, glioma. Non-Hodgkin's lymphoma, leukemia, multiple myeloma and multidrug resistant cancer. A “tumor” is an abnormal mass of tissue that results from excessive cell division that is uncontrolled and progressive and is also referred to as a neoplasm. The term “tumor,” as used herein, refers to all neoplastic cell growth and proliferation, whether malignant or benign, and all pre-cancerous and cancerous cells and tissues. In certain embodiments, the terms “cancer” or “tumor” are used interchangeably.

“Sample” as used herein means any biological fluid or suspension or tissue from a subject, including samples that contains cells carrying the LOX-1⁺ biomarker or PMN-MDSC signature biomarkers identified herein. The sample in one embodiment contains cells that are PMNs and PMN-MDSCs. The sample in one embodiment contains cells carrying one or more other biomarkers or cell surface antigens indicative of polymorphonuclear cells or neutrophils. In one embodiment, cells (neutrophils) in the sample express CD66b⁺. In another embodiment, cells (neutrophils) in the sample express CD15⁺. In still another embodiment, cells in the sample express CD11b⁺ or CD33⁺. The most suitable samples for use in the methods and with the diagnostic compositions or reagents described herein are samples or suspensions which require minimal invasion for testing, e.g., blood samples, including whole blood. It is anticipated that other biological samples that contain cells at a sufficiently detectable concentration, such as peripheral blood, serum, saliva or urine, vaginal or cervical secretions, and ascites fluids or peritoneal fluid may be similarly evaluated by the methods described herein. In one embodiment, the sample is a tumor secretome, i.e., any fluid or medium containing the proteins secreted from the tumor. These shed proteins may be unassociated, associated with other biological molecules, or enclosed in a lipid membrane such as an exosome. Also, tumor cells or fluids or tissues (for example, obtained from a biopsy) are also suitable samples for evaluation In certain embodiments of this invention. In another embodiment, the biological sample is a tissue or tissue extract, e.g., biopsied material, containing the PMN-MDSC. In certain embodiments, the biological sample is peripheral blood mononuclear cells (PBMC). In certain embodiments, the biological sample is neutrophil. In certain embodiments, such sample may be derived from a tissue biopsy. In one embodiment, such samples may further be diluted with or suspended in, saline, buffer or a physiologically acceptable diluent. Alternatively, such samples are tested neat. In another embodiment, the samples are concentrated by conventional means. In certain embodiments, the sample is obtained from a tumor microenvironment, for example, tissue, fluid, cell, immune cells, fibroblasts, sig haling molecules, extracellular matrix or other component around a tumor or a cancer.

In one embodiment, the biological sample is whole blood, and the method employs the PaxGene Blood RNA Workflow system (Qiagen). That system involves blood collection (e.g., single blood draws) and RNA stabilization, followed by transport and storage, followed by purification of Total RNA and Molecular RNA testing. This system provides immediate RNA stabilization and consistent blood draw volumes. The blood can be drawn at a physician's office or clinic, and the specimen transported and stored in the same tube. Short term RNA stability is 3 days at between 18-25° C. or 5 days at between 2-8° C. Long term RNA stability is 4 years at −20 to −70° C. This sample collection system enables the user to reliably obtain data on gene expression and miRNA expression in whole blood. In one embodiment, the biological sample is whole blood. While the PAXgene system has more noise than the use of peripheral blood mononuclear cells (PBMC) as a biological sample source, the benefits of PAXgene sample collection outweighs the problems. Noise can be subtracted bioinformatically.

The term “biomarker” as described in this specification includes any physiological molecular form, or modified physiological molecular form, isoform, pro-form, naturally occurring forms or naturally occurring mutated forms of a protein (for example, LOX-1) and peptide fragments thereof, expressed on the cell surface, in the cell, and/or in the cell cytosol, unless otherwise specified. Other biomarkers that may be useful to detect neutrophils to assist in distinguishing the two subsets PMN and PMN-MDSCs according to the teachings herein include CD66b, CD11b, CD33, CD15 and/or CD14 as well as the biomarkers that have been shown to be part of the PMN-MDSC signature, e.g., those listed in Table 1 in US Patent Application Publication with No. US20180059115. It is understood that all molecular forms useful in this context are physiological, e.g., naturally occurring in the species. Preferably the peptide fragments obtained from the biomarkers are unique sequences. However, it is understood that other unique fragments may be obtained readily by one of skill in the art in view of the teachings provided herein.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

By “related proteins” or “proteins of the same family” are meant expression products of different genes or related genes identified as belonging to a common family. Related proteins in the same biomarker family, e.g., LOX-1, may or may not share related functions. Related proteins can be readily identified as having significant sequence identity either over the entire protein or a significant part of the protein that is typically referred to as a “domain”. Proteins with at least 20% sequence homology or sequence identity can be readily identified as belonging to the same protein family.

By “homologous protein” is meant an alternative form of a related protein produced from a related gene having a percent sequence similarity or identity of greater than 20%, greater than 30%, greater than 40%, greater than 50%, greater than 60%, greater than 70%, greater than 75%, greater than 80%, greater than 85%, greater than 90%, greater than 95%, greater than 97%, or greater than 99%.

The term “ligand”, for example, with regard to protein biomarkers, refers to a molecule that binds or complexes, with the PMN-MDSC biomarker protein, e.g., LOX-1, CD15 or CD66b. Thus, a ligand can be an amino acid sequence or protein sequence, or a molecular form or peptide, such as an antibody, antibody mimic or equivalent, or a fragment thereof. The ligand can be a naturally occurring peptide that binds to a portion of a biomarker or a synthetically or recombinantly produced chimeric peptide having a portion that binds to the biomarker and a portion designed for other purposes, e.g., to assist in the detection of the binding. Similarly, the peptide may be designed, or a small molecule designed, to bind to a biomarker by mimicking the three-dimensional physical structure of the biomarker. The term ligand as used with respect to the neutrophil biomarkers, e.g., CD15 and CD66b, and the PMN-MDSC signature biomarkers identified herein refers to similar amino acid sequences, peptides, chimeric proteins, etc, which can bind with the respective cell proteins.

The term “ligand” with regarding to a nucleic acid sequence encoding a biomarker, refers to a molecule that binds or complexes, with the indicated biomarker nucleic acid, e.g., DNA or RNA of LOX-1, CD15 or CD66b. Such a ligand can itself be an antibody or antibody fragment, a nucleotide sequence, e.g., a polynucleotide or oligonucleotide, primer or probe, which can be complementary to the biomarker-encoding sequence.

As used herein, “labels” or “reporter molecules” or “detectable label components” are chemical or biochemical moieties that do not naturally occur in association with a ligand, but that are useful when manipulated into association with a ligand, that alone or in concert with other components enable the detection of a target. e.g., LOX-1 or another biomarker. Such labels or components include, without limitation, fluorescent agents, chemiluminescent agents, chromogenic agents, quenching agents, radionucleotides, enzymes, enzymatic substrates, cofactors, inhibitors, radioactive isotopes, magnetic particles, and other moieties known in the art. In certain embodiments, the “labels” or “reporter molecules” are covalently attached or associated with a ligand or an antibody or epitope binding fragment thereof. In certain other embodiments, the “labels” or “reporter molecules” are non-covalently attached or associated with the ligand. Such labels are capable of generating a measurable signal alone, e.g., radioactivity, or in association with another component, e.g., an enzymatic signal in the presence of a substrate.

In certain embodiments, a label may be a reagent capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

In certain embodiments, a label may be a detectable substance. Examples of detectable substances include, but are not limited to, the following: radioisotopes (e.g., 3H, 14C, 35S, 125I, 131I), fluorescent labels FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags).

As used herein, an antibody-drug conjugate or ADC refers to a complex molecule composed of an antibody or epitope binding fragment thereof conjugated to (covalently or non-covalently attached to or associated with) a potent cytotoxic agent, for example via chemical linkers. The cytotoxic agent induces target cell death after the antibody part of ADC directs it to a target cell. In certain embodiments, the cytotoxic agent is a small molecule drug with a high systemic toxicity. In certain embodiments, the cytotoxic agent is a chemotherapy drug (i.e., a drug that may be used in a chemotherapy for treating a cancer/tumor), for example, a biologically active anti-microtubule agent, an alkylating agent (such as duocarmycin derivatives such as CC-1065 analogs and duocarmycin), a DNA minor groove binding agent, an inhibitor of tubulin polymerization (such as the maytansinoids, dolastatins, auristatin drug analogues and cryptophycin), an enediyne antibiotic (including esperamicin and calicheamicin which catalyze DNA double-strand breaks and pyrrolobenzodiazepine (PBD)). In certain embodiments, the cytotoxic agent is a radioactive particle, such as an α-particle, or a β-particle such as ¹³¹I, ⁶⁷Cu, ¹⁷⁷Lu, and ⁹⁰Y. See, for example, Thaddeus J. Wadas et al., Molecular Targeted α-Particle Therapy for Oncologic Applications. Am J Roentgenol. 2014 August; 203(2): 253-260.

By “physical substrate” or “substrate” is meant a substrate upon which said polynucleotides or oligonucleotides or ligands or antibodies or epitope binding fragment thereof are immobilized. The physical substrate can be e.g., a glass slide, a plastic support, or a microchip. The term “microarray” refers to an ordered arrangement of binding/complexing array elements or ligands, e.g. antibodies, probes, etc. on a physical substrate. The substrates for immobilization may be any of the common substrates, glass, plastic, a microarray, a microfluidics card, a chip or a chamber. Suitable carriers or supports may comprise nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip) Immobilization typically entails separating the binding agent from any free analytes (e.g. free markers or free complexes thereof) in the reaction mixture.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides. RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

A “nucleic acid”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

A “vector” as used herein is a biological or chemical moiety comprising a nucleic acid sequence which can be introduced into an appropriate host cell for replication or expression of the nucleic acid sequence. Common vectors include naked DNA, phage, transposon, plasmids, viral vectors, cosmids (Phillip McClean, www.ndsu.edu/pubweb/˜mcclean/plsc731/cloning/cloning4.htm) and artificial chromosomes (Gong, Shiaoching, et al. “A gene expression atlas of the central nervous system based on bacterial artificial chromosomes.” Nature 425.6961 (2003): 917-925). One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host ceil upon introduction into the host cell, and thereby are replicated along with the host genome. Moreover, certain vectors are capable of directing the expression of genes to which they are operatively linked.

A “viral vector” refers to a synthetic or artificial viral particle in which an expression cassette containing a nucleic acid sequence of interest is packaged in a viral capsid or envelope. Examples of viral vector include but are not limited to adenoviruses (Ads), retroviruses (γ-retroviruses and lentiviruses), poxviruses, adeno-associated viruses (AAV), baculoviruses, herpes simplex viruses. In one embodiment, the viral vector is replication defective. A “replication-defective virus” refers to a viral vector, wherein any viral genomic sequences also packaged within the viral capsid or envelope are replication-deficient; i.e., they cannot generate progeny virions but retain the ability to infect target cells.

In certain embodiments, the vector is a viral vector selected from a recombinant parvovirus, a recombinant lentivirus, a recombinant retrovirus, or a recombinant adenovirus; or a non-viral vector selected from naked DNA, naked RNA, an inorganic particle, a lipid particle, a polymer-based vector, or a chitosan-based formulation. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

The term “regulatory element” or “regulatory sequence” refers to expression control sequences which are contiguous with the nucleic acid sequence of interest and expression control sequences that act in trans or at a distance to control the nucleic acid sequence of interest. As described herein, regulatory elements comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Also, see Goeddel; Gene Expression Technology: Methods in Enzymology 185, Academic Press, San Diego, Calif. (1990). Regulatory sequences include those which direct constitutive expression of a nucleic acid sequence in many types of target cell and those which direct expression of the nucleic acid sequence only in certain target cells (e.g., tissue-specific regulatory sequences). It will be appreciated by those skilled in the art that the design of the vector can depend on such factors as the choice of the host cell, the level of expression desired, and the like.

In certain embodiments, the terms “production cell” and “host cell”, which are used herein interchangeably, may refer to any target cell to which introduction of the nucleic acid sequence or vector of interest is desired. Thus, a “host cell,” refers to a cell that contains the nucleic acid sequence of interest that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species. In one embodiment; the host cell is a mammalian cell. In a further embodiment, the target cell might be a eukaryotic cell, a prokaryotic cell, an embryonic stem cell, a cancer cell, a neuronal cell, an epithelial cell, an immune cell, an endocrine cell, a muscle cell, an erythrocyte, a lymphocyte, a CNS cell (for example, a neuron), a PNS cell, or another cell (such as a kidney cell, or a liver cell). In certain embodiments, a host cell refers to a cell in a subject or patient. In certain embodiments, a host cell refers to a cell which is able to produce a peptide or a protein (for example, an antibody or epitope binding fragment thereof as described herein). In certain embodiments, a host cell is a hybridoma. As used herein, a hybridoma is a hybrid cell used as the basis for the production of antibodies in large amounts for diagnostic or therapeutic use. Hybridomas are produced by fusing an antibody-producing cell with an immortal cell (such as a b cell cancer cell, a myeloma cell).

As used herein, a signal peptide (sometimes referred to as signal sequence, targeting signal, localization signal, localization sequence, transit peptide, leader sequence or leader peptide) is a short peptide (usually 15-30 amino acids long) present at the N-terminus of the majority of newly synthesized proteins that are destined towards the secretory pathway (Blobel G, Dobberstein B., “Transfer of proteins across membranes. I. Presence of proteolytically processed and unprocessed nascent immunoglobulin light chains on membrane-bound ribosomes of murine myeloma”. J Cell Biol. December 1975, 67 (3): 835-51). These proteins include those that reside either inside certain organelles (the endoplasmic reticulum, golgi or endosomes), secreted from the cell, or inserted into most cellular membranes. In certain embodiments, the signal peptide is an IgE signal peptide (leader sequence) that has an amino acid sequence of aa 1 to aa 18 of SEQ ID NO: 1. In certain embodiments, the signal peptide is from a protein which is secreted by a CNS cell (for example, a neuron), a PNS cell, or another cell (such as a kidney cell, or a liver cell). The signal peptide is preferably of human origin or a derivative of a human signal peptide, and is about 15 to about 30 amino acids, preferably about 17 to 25 amino acids, or about 18 amino acids in length. Other suitable signal peptides may be selected by one of skills in the art.

As used herein, the terms “therapy”, “treatment” and any grammatical variations thereof shall mean any of prevention, delay of outbreak, reducing the severity of the disease symptoms, and/or removing the disease symptoms (to cure) in a subject in need. In certain embodiments, the term “treatment” or any grammatical variations thereof refer to reducing or inhibiting the growth or metastasis of cancer(s), slowing or inhibiting the progression of a cancer, delaying or preventing cancer remission, reducing or inhibiting the growth or metastasis of cancer cells that express LOX-1, reducing the growth, spread or amount of LOX-1+ cells and/or the LOX-1 expression level on/in a cell (for example, in any disorder characterized by cells expressing LOX-1), and/or retarding, suppressing or inhibiting the PMN-′MSC.

By the terms “increase” “decrease” “inhibit” “change” “slow” “reduce” “delay” “prevent” “suppress” “enhance” or a grammatical variation thereof, refer to a variability of at least about 10% from the reference given, unless otherwise specified. By the tears “low” “high” or a grammatical variation thereof, refer to a variability of at least about 10%, or at least about 20%, or at least about 30%, or at least about 40%, or at least about 50%, or at least about 75%, or at least about 80%, or at least about 90%, from the reference given, unless otherwise specified.

By “pharmaceutically acceptable carrier or excipient” is meant a solid and/or liquid carrier, in in dry or liquid form, which is pharmaceutically acceptable. The compositions are typically sterile solutions or suspensions. Examples of excipients which may be combined with the antagonist or inhibitor include, without limitation, solid carriers, liquid carriers, adjuvants, amino acids (glycine, glutamine, asparagine, arginine, lysine), antioxidants (ascorbic acid, sodium sulfite or sodium hydrogen-sulfite), binders (gum tragacanth, acacia, starch, gelatin, polyglycolic acid, polylactic acid, poly-d,l-lactide/glycolide, polyoxaethylene, polyoxapropylene, polyacrylamides, polymaleic acid, polymaleic esters, polymaleic amides, polyacrylic acid, polyacrylic esters, polyvinylalcohols, polyvinylesters, polyvinylethers, polyvinylimidazole, polyvinylpyrrolidon, or chitosan), buffers (borate, bicarbonate, Tris-HCl, citrates, phosphates or other organic acids), bulking agents (mannitol or glycine), carbohydrates (such as glucose, mannose, or dextrins), clarifiers, coatings (gelatin, wax, shellac, sugar or other biological degradable polymers), coloring agents, complexing agents (caffeine, polyvinylpyrrolidone, β-cyclodextrin or hydroxypropyl-β-cyclodextrin), compression aids, diluents, disintegrants, dyes, emulsifiers, emollients, encapsulating materials, fillers, flavoring agents (peppermint or oil of wintergreen or fruit flavor), glidants, granulating agents, lubricants, metal chelators (ethylenediamine tetraacetic acid (EDTA)), osmo-regulators, pH adjustors, preservatives (benzalkonium chloride, benzoic acid, salicylic acid, thimerosal, phenethyl alcohol, methylparaben, propylparaben, chlorhexidine, sorbic acid, hydrogen peroxide, chlorobutanol, phenol or thimerosal), solubilizers, sorbents, stabilizers, sterilizer, suspending agent, sweeteners (mannitol, sorbitol, sucrose, glucose, mannose, dextrins, lactose or aspartame), surfactants, syrup, thickening agents, tonicity enhancing agents (sodium or potassium chloride) or viscosity regulators. See, the excipients in “Handbook of Pharmaceutical Excipients”, 5^{th} Edition, Eds.: Rowe, Sheskey, and Owen, APhA Publications (Washington, D.C.), 2005 and U.S. Pat. No. 7,078,053, which are incorporated herein by reference. The selection of the particular excipient is dependent on the nature of the compound selected and the particular form of administration desired.

Solid carriers include, without limitation, starch, lactose, dicalcium phosphate, microcrystalline cellulose, sucrose and kaolin, calcium carbonate, sodium carbonate, bicarbonate, lactose, calcium phosphate, gelatin, magnesium stearate, stearic acid, or talc. Fluid carriers without limitation, water, e.g., sterile water, Ringer's solution, isotonic sodium chloride solution, neutral buffered saline, saline mixed with serum albumin, organic solvents (such as ethanol, glycerol, propylene glycol, liquid polyethylene glycol, dimethylsulfoxide DMSO)), oils (vegetable oils such as fractionated coconut oil, arachis oil, corn oil, peanut oil, and sesame oil; oily esters such as ethyl oleate and isopropyl myristate; and any bland fixed oil including synthetic mono- or diglycerides), fats, fatty acids (include, without limitation, oleic acid find use in the preparation of injectables), cellulose derivatives such as sodium carboxymethyl cellulose, and/or surfactants.

By “effective amount” is meant the amount or concentration (by single dose or in a dosage regimen delivered per day) of the antibody or epitope binding fragment or antagonist or inhibitor or any agent referred to sufficient to reduce or inhibit the growth or metastasis of cancer(s); slow or inhibit the progression of a cancer; delay or prevent cancer remission; reduce or inhibit the growth or metastasis of cancer cells (including cancer cells that express LOX-1); reduce the growth, spread or amount of LOX-1+ cells and/or the LOX-1 expression level on/in a cell (for example, in any disorder characterized by cells expressing LOX-1); and/or retard, suppress or inhibit the PMN-MDSC, while providing the least negative side effects to the treated subject. One of skill in the art would be able to determine the amount to administer alone or in combination with an additional reagent, e.g., chemotherapeutic, antibiotic or the like. In a further embodiment, the combination with another pharmacological agent or treatment protocol permits lower than usual amounts of the agonist and additional chemotherapeutic agent to achieve the desired therapeutic effect. In another embodiment, the combination with another chemotherapy treatment protocol permits adjustment of the additional protocol regimen to achieve the desired therapeutic effect.

In one embodiment, the effective amount is within the range of 1 mg/kg body weight to 100 mg/kg body weight in humans including all integers or fractional amounts within the range. In certain embodiments, the effective amount is at least 2, 5, 10, 15, 20, 25, 30, 35, 40, 45, 50, 55, 60, 65, 70, 75, 80, 85, 90, 95 or 100 mg/kg body weight, including all integers or fractional amounts within the range. In one embodiment, the above amounts represent a single dose. In another embodiment, the above amounts define an amount delivered to the subject per day. In another embodiment, the above amounts define an amount delivered to the subject per day in multiple doses. In still other embodiments, these amounts represent the amount delivered to the subject over more than a single day.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also be described using “consisting of” or “consisting essentially of” language.

The term “a” or “an”, refers to one or more, for example, “an antibody,” is understood to represent one or more antibodies. As such, the terms “a” (or “an”), “one or more,” and “at least one” are used interchangeably herein.

As used herein, the term “about” means a variability of 10% from the reference given, unless otherwise specified.

### Coding Sequence and Recombinant Protein of LOX-1

A recombinant human LOX-1 (hLOX-1) protein is provided herein. See, for example, SEQ ID NO: 1. An IgE signal peptide (i.e., leader sequence) of amino acid (aa) 1 to aa 18 of SEQ ID NO: 1) is fused to the N terminal of human LOX-1 protein in order to facilitate its expression in a host cell. In certain embodiments, the hLOX-1 coding sequence is further covalently or non-covalently attached to or associated with (conjugated to) a detectable label and/or a coding sequence for a detectable label, for example, a His tag.

Further provided is a nucleic acid sequence encoding the recombinant hLOX-1 protein or an amino acid sequence which is at least 85% identical thereto. The hLOX-1 coding sequence may be engineered for enhanced expression, such as SEQ ID NO: 28 or a nucleic acid sequence at least 85% identical thereto.

In certain embodiments, provided herein is a vector comprising hLOX-1 coding sequence, for example, SEQ ID NO: 28. The vector may further comprise regulatory sequences which direct expression of the hLOX-1 protein in a host cell. In certain embodiments, the regulatory sequences comprise a promoter, an optional enhancer, an optional intron, and an optional polyadenylation sequence (polyA).

In certain embodiments, the host cell is a cell in a subject, for example, a mouse, a rabbit, a goat, a donkey, or a camelid. In one embodiment, the vector is a plasmid. In certain embodiments, the vector comprises a cytomegalovirus immediate-early (CMV) promoter. Additionally, or alternatively, the vector comprises a bovine growth hormone (BGH) polyA.

In certain embodiments, the host cell is an Escherichia coli cell. In one embodiment, the vector is a plasmid. In certain embodiments, the regulator sequences comprise a LacI promoter.

Also provided herein is a host cell expressing the recombinant hLOX-1 protein. In certain embodiments, the host cell comprises a vector comprising a hLOX-1 coding sequence as described herein. In certain embodiments, the host cell is a hybridoma cell. In certain embodiments, the host cell is provided for producing an antibody or an epitope binding fragment thereof as described herein in a large scale.

### LOX-1 Antibody (Anti-LOX-1 Antibody)

In one aspect, provided herein is a recombinant antibody or an epitope binding fragment thereof that specifically binds to a lectin-like oxidized low-density lipoprotein (LDL) receptor-1 (LOX-1) epitope. Such antibody or fragment thereof is also referred to as a LOX-1 antibody or an anti-LOX-1 antibody as used herein.

In certain embodiments, the LOX-1 epitope is in the extracellular region of LOX-1 (for example, amino acid (aa) 75 to aa 290 of SED ID NO: 1). In certain embodiments, the LOX-1 epitope is in the C-type lectin-like domain (CTLD) domain of LOX-1 (for example, aa 160 to aa 290 of SEQ ID NO: 1). In certain embodiments, the LOX-1 epitope is in the cytoplasmic domain of LOX-1 (for example, aa 19 to aa 53 of SEQ ID NO: 1). In certain embodiments, the LOX-1 epitope is not in the transmembrane domain of LOX-1 (for example, aa 54 to aa 74 of SEQ ID NO: 1). In certain embodiments, the epitope is a conformation sensitive epitope which only presents in a native conformation. In certain embodiments, the epitope only presents when the LOX-1 protein is in its native conformation, i.e., is folded properly into a three-dimensional shape which is the same as in vivo in a healthy subject. In certain embodiments, the epitope only presents when the LOX-1 protein is expressed in a cell or on a cell surface thereof, for example, a neutrophil or a PMN.

In certain embodiments, the antibody or epitope binding fragment thereof specifically recognizes and binds to an epitope of LOX-1 protein in native condition (such as, a conformation sensitive epitope). In certain embodiments, the antibody or epitope binding fragment thereof recognizes and binds to an epitope of LOX-1 protein in denatured condition. In certain embodiments, the antibody or epitope binding fragment thereof is able to specifically recognize and hind to an epitope of LOX-1 protein in native condition as well as an epitope of LOX-1 protein in denatured condition.

In certain embodiments, the antibody or epitope binding fragment thereof comprises at least one of complementarity-determining regions (CDRs) of 3D8, 6A10, 9E12, 12D9, 12E4 or 4D6. In a further embodiment, the CDR(s) may be a heavy chain CDR and/or a light chain CDR. n certain embodiments, the antibody or epitope binding fragment thereof comprises CDRs 1-3 of the 3D8 heavy chain. In certain embodiments, the antibody or epitope binding fragment thereof comprises CDRs 1-3 of the 3D8 light chain. In certain embodiments, the antibody or epitope binding fragment thereof comprises CDRs 1-3 of the 3D8 light chain and CDRs 1-3 of the 3D8 heavy chain. In certain embodiments, the antibody or epitope binding fragment thereof comprises CDRs 1-3 of the 6A10 heavy 1.5 chain. In certain embodiments, the antibody or epitope binding fragment thereof comprises CDRs 1-3 of the CA 10 light chain. In certain embodiments, the antibody or epitope binding fragment thereof comprises CDRs 1-3 of the 6A10 light chain and CDRs 1-3 of the 6A10 heavy chain. In certain embodiments, the antibody or epitope binding fragment thereof comprises CDRs 1-3 of the 9E12 heavy chain. In certain embodiments, the antibody or epitope binding fragment thereof comprises CDRs 1-3 of the 9E12 light chain. In certain embodiments, the antibody or epitope binding fragment thereof comprises CDRs 1-3 of the 9E12 light chain and CDRs 1-3 of the 9E12 heavy chain. In certain embodiments, the antibody or epitope binding fragment thereof comprises CDRs 1-3 of the 12D9 heap chain. In certain embodiments, the antibody or epitope binding fragment thereof comprises CDRs 1-3 of the 12D9 light chain. In certain embodiments, the antibody or epitope binding fragment thereof comprises CDRs 1-3 of the 12D9 light chain and CDRs 1-3 of the 12D9 heavy chain. In certain embodiments, the antibody or epitope binding fragment thereof comprises CDRs 1-3 of the 12E4 heavy chain. In certain embodiments, the antibody or epitope binding fragment thereof comprises CDRs 1-3 of the 12E4 light chain. In certain embodiments, the antibody or epitope binding fragment thereof comprises CDRs 1-3 of the 12E4 light chain and CDRs 1-3 of the 12E4 heavy chain. In certain embodiments, the antibody or epitope binding fragment thereof comprises CDRs 1-3 of the 4D6 heavy chain. In certain embodiments, the antibody or epitope binding fragment thereof comprises CDRs 1-3 of the light chain of 4D6 clone 1. In certain embodiments, the antibody or epitope binding fragment thereof comprises CDRs 1-3 of the light chain of 4D6 clone 1 and CDRs 1-3 of the 6A10 heavy chain. In certain embodiments, the antibody or epitope binding fragment thereof comprises CDRs 1-3 of the light chain of 4D6 clone 2. In certain embodiments, the antibody or epitope binding fragment thereof comprises CDRs 1-3 of the light chain of 4D6 clone 2 and CDRs 1-3 of the 6A10 heavy chain. In certain embodiments, the antibody or epitope binding fragment thereof may comprise CDRs 1-3 of a heavy chain of 3D8, 6A10, 9E12, 12D9, 12E4 or 4D6 and CDRs 1-3 of a light chain of 3D8, 6A10, 9E12, 12D9, 12E4 or 4D6. In certain embodiments, the antibody or epitope binding fragment thereof may comprise CDRs 1-3 of a heavy chain of 3D8, 6A 10, 9E12, 12D9, 12E4 or 4D6 and CDRs 1-3 of a light chain other than 3D8, 6A10, 9E12, 12D9, 12E4 or 4D6. In certain embodiments, the anti-LOX1 antibody or epitope binding fragment thereof may comprise a light chain variable region which does not recognize LOX-1. In certain embodiments, the antibody or epitope binding fragment thereof may further comprise a CDR or CDRs specifically recognizing an epitope other than LOX-1 protein, for example, a biomarker for neutrophil or PMN, such as CD15 or CD66b.

As used herein, In certain embodiments, a CDR is encoded by a CDR-coding nucleic acid sequence as provided in the tables and paragraphs below, or a nucleic acid sequence at least 85%, 86%, 87%, 88%, 89%, 90%, 91%, 92%, 93%, 94%, 95%, 96%, 97%, 98%, 99%, up to 100% identical thereto. Additionally or alternatively, a CDR has a CDR amino acid sequence as provided in the tables and paragraphs below, or a modification thereof, and/or an amino acid sequence at least 85%, 86%, 87%, 88%, 89%, 90%, 91%, 92%, 93%, 94%, 95%, 96%, 97%, 98%, 99%, up to 100% identical thereto.

TABLE 1
Coding Sequences for Heavy Chain CDRs

| | CDR | Nucleic Acid | |
|---|---|---|---|
| Aby | No. | Sequence | SEQ ID NO |
| 6A10 | 1 | GGC TAC TAC ATG | nt 148-162 of |
| | | CAC | SEQ ID NO: 2 |
| | 2 | CGT ATT AAT CCT | nt 205-255 of |
| | | TAC AAT GGT | SEQ ID NO: 2 |
| | | GCT ACT AGC TAC | |
| | | AAC CAG AAT | |
| | | TTC AAG GAC | |
| | 3 | GAC GTC GCT TAC | nt 352-363 of |
| | | | SEQ ID NO: 2 |
| 9E12 | 1 | AGC TAC TGG ATG | nt 148-162 of |
| | | CAC | SEQ ID NO: 6 |
| | 2 | ACG ATT GAT CCT | nt 205-255 of |
| | | TCA GAT AGT | SEQ ID NO: 6 |
| | | TAT ACT AGC TAC | |
| | | AAT CAA AAG | |
| | | TTC AAG GGC | |
| | 3 | TTT GAC TAC | nt 352-360 of |
| | | | SEQ ID NO: 6 |
| 12D9 | 1 | AAC TAT GGA ATG | nt 148-162 of |
| | | AAC | SEQ ID NO: 10 |
| | 2 | TGG ATA AAC ACC | nt 205-255 of |
| | | TAC ACT GGA | SEQ ID NO: 10 |
| | | GAG CCA ACA TAT | |
| | | ACT GAT GAC | |
| | | TTC AAG GGA | |
| | 3 | GGG GGC GAT TTC | nt 352-378 of |
| | | TAT GCT ATG | SEQ ID NO: 10 |
| | | GAC TAC | |
| 12E4 | 1 | GGC TAC ACC ATG | nt 148-162 of |
| | | AAC | SEQ ID NO: 14 |
| | 2 | CTT ATT AAT CCT | nt 205-255 of |
| | | TAC AAT GGT | SEQ ID NO: 14 |
| | | GGT ACT AGC TAC | |
| | | AAC CAG AAG | |
| | | TTC AAG GGC | |
| | 3 | GGG GGC TTC TAT | nt 352-375 of |
| | | GCT ATG GAC | SEQ ID NO: 14 |
| | | TAC | |
| 4D6 | 1 | AGC TAC TGG ATA | nt 148-162 of |
| | | GAG | SEQ ID NO: 18 |
| | 2 | GAG ATT TTA CCT | nt 205-255 of |
| | | GGA AGT GAT | SEQ ID NO: 18 |
| | | AGT ACT TAC TAC | |
| | | AAT GAG AAC | |
| | | TTC AAG GGC | |
| | 3 | GAC AGC TCG GGC | nt 352-375 of |
| | | TTG TTT GCT | SEQ ID NO: 18 |
| | | TAC | |
| 3D8 | 1 | AGC TAT GTT ATG | nt 148-162 of |
| | | CAC | SEQ ID NO: 24 |
| | 2 | TAT ATT AAT CCT | nt 205-255 of |
| | | TAC AAT GAT | SEQ ID NO: 24 |
| | | GGT ACT AAG TAC | |
| | | AAT GAG AAG | |
| | | TTC AAA GGC | |
| | 3 | GGG GTC TAT GAT | nt 352-378 of |
| | | GGT TAC CCT | SEQ ID NO: 24 |
| | | GAC TAC | |

TABLE 2
Amino Acid Sequences of
Heavy Chain CDRs

| | CDR | | |
|---|---|---|---|
| Aby | No. | Amino Acid Sequence | SEQ ID NO |
| | 1 | GYYMH | aa 50-54 of |
| | | | SEQ ID NO: 3 |
| 6A10 | 2 | RINPYNGATSYNQNFKD | aa 69-85 of |
| | | | SEQ ID NO: 3 |
| | 3 | DVAY | aa 118-121 of |
| | | | SEQ ID NO: 3 |
| 9E12 | 1 | SYWMH | aa50-54 of |
| | | | SEQ ID NO: 7 |
| | 2 | TIDPSDSYTSYNQKFKG | aa 69-85 of |
| | | | SEQ ID NO: 7 |
| | 3 | FDY | aa 118-120 of |
| | | | SEQ ID NO: 7 |
| 12D9 | 1 | NYGMN | aa 50-54 of |
| | | | SEQ ID NO: 11 |
| | 2 | WINTYTGEPTYTDDFKG | aa 69-85 of |
| | | | SEQ ID NO: 11 |
| | 3 | GGDFYAMDY | aa 118-126 of |
| | | | SEQ ID NO: 11 |
| 12E4 | 1 | GYTMN | aa 50-54 of |
| | | | SEQ ID NO: 15 |
| | 2 | LINPYNGGTSYNQKFKG | aa69-85 of |
| | | | SEQ ID NO: 15 |
| | 3 | GGFYAMDY | aa 118-125 of |
| | | | SEQIDNO: 15 |
| 4D6 | 1 | SYWIE | aa 50-54 of |
| | | | SEQ ID NO: 19 |
| | 2 | EILPGSDSTYYNENFKG | aa 69-85 of |
| | | | SEQ ID NO: 19 |
| | 3 | DSSGLFAY | aa 118-125 of |
| | | | SEQ ID NO: 19 |
| 3D8 | 1 | SYVMH | aa50-54 of |
| | | | SEQ ID NO: 25 |
| | 2 | YINPYNDGTKYNEKFKG | aa 69-85 of |
| | | | SEQ ID NO: 25 |
| | 3 | GVYDGYPDY | aa 118-126 of |
| | | | SEQ ID NO: 25 |

TABLE 3
Coding Sequences for Light Chain CDRs

| | CDR | | |
|---|---|---|---|
| Aby | No. | Nucleic Acid Sequence | SEQ ID NO |
| 6A10 | 1 | AAG GCG AGT CAG | nt 130-162 of |
| | | GAC ATT AAT | SEQ ID NO: 4 |
| | | AGC TAT TTA AGC | |
| | 2 | CGT GCA AAC AGA | nt 208-228 of |
| | | TTG GTA GAT | SEQ ID NO: 4 |
| | 3 | CTA CAG TAT GAT | nt 325-354 of |
| | | GAG TTT CCT | SEQ ID NO: 4 |
| | | CCG TGG ACG | |
| 9E12 | 1 | AAG TCA AGT CAG | nt 127-174 of |
| | | AGC CTC TTA | SEQ ID NO: 8 |
| | | GAT AGT GAT GGA | |
| | | AAG ACA TAT | |
| | | TTG AAT | |
| | 2 | CTG GTG TCT AAA | nt 220-240 of |
| | | CTG GAC TCT | SEQ ID NO: 8 |
| | 3 | TGG CAA GGT ACA | nt 337-363 of |
| | | CAT TTT CCA | SEQ ID NO: 8 |
| | | TTC ACG | |
| 12D9 | 1 | AAG GCC AGT GAG | nt 130-162 of |
| | | AAT GTG GGT | SEQ ID NO: 12 |
| | | ACT TAT GTA TCC | |
| | 2 | GGG GCA TCC AAC | nt 208-228 of |
| | | CGG AAC ACT | SEQ ID NO: 12 |
| | 3 | GGA CAG AGT TCC | nt 325-351 of |
| | | AAC TAT CCA | SEQ ID NO: 12 |
| | | TAC ACG | |
| 12E4 | 1 | AGT GCA AGT CAG | nt 127-159 of |
| | | GGC ATT AGC | SEQ ID NO: 16 |
| | | AAT TAT TTA AAC | |
| | 2 | TAC ACA TCA AGT | nt 205-225 of |
| | | TTA CAC TCA | SEQ ID NO: 16 |
| | 3 | CAG CAG TAT AGT | nt 322-348 of |
| | | AAG CTT CCG | SEQ ID NO: 16 |
| | | TAC ACG | |
| 4D6, | 1 | AAG GCC AGT CAG | nt 130-162 of |
| clone | | AGT GTG AGT | SEQ ID NO: 20 |
| 1 | | AAT GAT GTA GCT | |
| | 2 | TAT GCA TCC AAT | nt 208-228 of |
| | | CGC TAC ACT | SEQ ID NO: 20 |
| | 3 | CAG CAG GAT TAT | nt 325-351 of |
| | | AGC TCT CCG | SEQ ID NO: 20 |
| | | CTC ACG | |
| 4D6, | 1 | CAA GCC AGC GAA | nt 130-174 of |
| clone | | AGT GTC AGT | SEQ ID NO: 22 |
| 2 | | TTT GCT GAT ACA | |
| | | AGT TTA ATG | |
| | | CAC | |
| | 2 | CGT GCA TCC AAC | nt 220-240 of |
| | | CTA GAA TCT | SEQ ID NO: 22 |
| | 3 | ATG CAA AGT ATG | nt 337-363 of |
| | | GAA GAT CCA | SEQ ID NO: 22 |
| | | TTC ACG | |
| 3D8 | 1 | AGG GCC AGC CAG | nt 130-162 of |
| | | AGT ATT AGC | SEQ ID NO: 26 |
| | | GAC TAC TTA CAC | |
| | 2 | TAT GCT TCC CAA | nt 208-228 of |
| | | TCC ATC TCT | SEQ ID NO: 26 |
| | 3 | CAA AAT GGT CAC | nt 325-351 of |
| | | AGC TTT CCG | SEQ ID NO: 26 |
| | | TAC ACG | |

TABLE 4
Amino Acid Sequences of Light Chain CDRs

| | CDR | Amino Acid | |
|---|---|---|---|
| Aby | No. | Sequence | SEQ ID NO |
| 6A10 | 1 | KASQDINSYLS | aa 44-54 of |
| | | | SEQ ID NO: 5 |
| | 2 | RANRLVD | aa 70-76 of |
| | | | SEQ ID NO: 5 |
| | 3 | LQYDEFPPWT | aa 109-118 of |
| | | | SEQ ID NO: 5 |
| 9E12 | 1 | KSSQSILDSDGKTYLN | aa 43-58 of |
| | | | SEQ ID NO: 9 |
| | 2 | LVSKLDS | aa 74-80 of |
| | | | SEQ ID NO: 9 |
| | 3 | WQGTHFPFT | aa 113-121 of |
| | | | SEQ ID NO: 9 |
| 12D9 | 1 | KASENVGTYVS | aa 44-54 of |
| | | | SEQ ID NO: 13 |
| | 2 | GASNRNT | aa 70-76 of |
| | | | SEQ ID NO: 13 |
| | 3 | GQSSNYPYT | aa 109-117 of |
| | | | SEQ ID NO: 13 |
| 12E4 | 1 | SASQGISNYLN | aa 43-53 of |
| | | | SEQ ID NO: 17 |
| | 2 | YTSSLHS | aa 69-75 of |
| | | | SEQ ID NO: 17 |
| | 3 | QQYSKLPYT | aa 108-116 of |
| | | | SEQ ID NO: 17 |
| 4D6, | 1 | KASQSVSNDVA | aa 44-54 of |
| clone | | | SEQ ID NO: 21 |
| 1 | | | |
| | 2 | YASNRYT | aa 70-76 of |
| | | | SEQ ID NO: 21 |
| | 3 | QQDYSSPLT | aa 109-117 of |
| | | | SEQ ID NO: 21 |
| 4D6, | 1 | QASESVSFADTSLMH | aa 44-58 of |
| clone | | | SEQ ID NO: 23 |
| 2 | | | |
| | 2 | RASNLES | aa 74-80 of |
| | | | SEQ ID NO: 23 |
| | 3 | MQSMEDPFT | aa 113-121 of |
| | | | SEQ ID NO: 23 |
| 3D8 | 1 | RASQS1SDYLH | aa 44-54 of |
| | | | SEQ ID NO: 27 |
| | 2 | YASQSI | aa 70-76 of |
| | | | SEQ ID NO: 27 |
| | 3 | QNGHSFPYI | aa 109-117 of |
| | | | SEQ ID NO: 27 |

TABLE 5
Sequences of Clones 6A10, 9E12, 12D9, 12E4, 4D6 (clones 1 and 2) and 3D8
6A10 Heavy Chain Variable Region
V-GENE and allele: Musmus IGHV1-26*01 F,
or Musmus IGHV1-34*02 F
J-GENE and allele: Musmus IGHJ3*01 F

| nucleic | Signal sequence-FR1-CDR1-FR2-CDR2-FR3-CDR3-FR4 | | SEQ ID NO: 2 |
|---|---|---|---|
| acid | ATGGGATGGAGCTGGATCTTTCTCTTTCTCCTGTCA | | |
| sequences | GGAACTGCAGGTGTCCTCTCTGAGGTCCAGCTGCA | | |
| | ACAGTCTGGACCTGAGCTGGTGAAGCCTGGGGCTT | | |
| | CAGTGAAGATATCCTGCAAGGCTTCTGGTTACTCA | | |
| | TTCACTGGCTACTACATGCACTGGGTGAAGCAAAG | | |
| | CCATGTAAAGAGCCTTGAGTGGATTGGACGTATTA | | |
| | ATCCTTACAATGGTGCTACTAGCTACAACCAGAAT | | |
| | TTCAAGGACAAGGCCAGCTTGACTGTAGATAAGTC | | |
| | CTCCAGCACAGCCTACATGGAGCTCCACAGCCTGA | | |
| | CATCTGAGGACTCTGCAGTCTATTACTGTGCCCCC | | |
| | GACGTCGCTTACTGGGGCCAAGGGACTCTGGTCAC | | |
| | TGTCTCTGCA | | |
| | Signal | ATGGGATGGAGCTGGATCTTTCTCTTTCT | nt 1-57 of |
| | seq | CCTGTCAGGAACTGCAGGTGTCCTCTCT | SEQ ID NO: 2 |
| | FR1 | GAGGTCCAGCTGCAACAGTCTGGACCTG | nt 58-147 of |
| | | AGCTGGTGAAGCCTGGGGCTTCAGTGAA | SEQ ID NO: 2 |
| | | GATATCCTGCAAGGCTTCTGGTTACTCAT | |
| | | TCACT | |
| | CDR1 | GGCTACTACATGCAC | nt 148-162 of |
| | | | SEQ ID NO: 2 |
| | FR2 | TGGGTGAAGCAAAGCCATGTAAAGAGCC | nt 163-204 of |
| | | TTGAGTGGATTGGA | SEQ ID NO: 2 |
| | CDR2 | CGTATTAATCCTTACAATGGTGCTACTAG | nt 205-255 of |
| | | CTACAACCAGAATTTCAAGGAC | SEQ ID NO: 2 |
| | FR3 | AAGGCCAGCTTGACTGTAGATAAGTCCT | nt 256-351 of |
| | | CCAGCACAGCCTACATGGAGCTCCACAG | SEQ ID NO: 2 |
| | | CCTGACATCTGAGGACTCTGCAGTCTATT | |
| | | ACTGTGCCCCC | |
| | CDR3 | GACGTCGCTTAC | nt 352-363 of |
| | | | SEQ ID NO: 2 |
| | FR4 | TGGGGCCAAGGGACTCTGGTCACTGTCTC | nt 364-396 of |
| | | TGCA | SEQ ID NO: 2 |
| amino | Signal sequence-FR1-CDR1-FR2-CDR2-FR3-CDR3-FR4 | | SEQ ID NO: 3 |
| acid | MGWSWIFLFLLSGTAGVLSEVQLQQSGPELVKPGAS | | |
| sequences | VKISCKASGYSFTGYYMHWVKQSHVKSLEWIGRiNP | | |
| | YNGATSYNQNFKDKASLTVDKSSSTAYMELHSLTSE | | |
| | DSAVYYCAPDVAYWGQGTLVTVSA | | |
| | Signal | MGWSWIFLFLLSGTAGVLS | aa 1-19 of |
| | seq | EVQLQQSGPELVKPGASVKISCKASGYSFT | SEQ ID NO: 3 |
| | FR1 | | aa 20-49 of |
| | | | SEQ ID NO: 3 |
| | CDR1 | GYYMH | aa 50-54 of |
| | | | SEQ ID NO: 3 |
| | FR2 | WVKQSHVKSLEWIG | aa 55-68 of |
| | | | SEQ ID NO: 3 |
| | CDR2 | R1NPYNGATSYNQNFKD | aa 69-85 of |
| | | | SEQ ID NO: 3 |
| | FR3 | KASLTVDKSSSTAYMELHSLTSEDSAVYYC | aa 86-117 of |
| | | AP | SEQ ID NO: 3 |
| | CDR3 | DVAY | aa 118-121 of |
| | | | SEQ ID NO: 3 |
| | FR4 | WGQGTLVTVSA | aa 122-132 of |
| | | | SEQ ID NO: 3 |

6A10 light chain variable region (IgM, Kappa)
V-GENE and allele: Musmus IGKV14-111*01 F
J-GENE and allele: Musmus IGKJ1*01 F

| nucleic | Signal sequence-FR1-CDR1-FR2-CDR2-FR3-CDR3-FR4 | | SEQ ID NO: 4 |
|---|---|---|---|
| acid | ATGAGGACCCCTGCTCAGTTTCTTGGAATCTTGTT | | |
| sequences | GCTCTGGTTTCCAGGTATCAAATGTGACATCAAGA | | |
| | TGACCCAGTCTCCATCTTCCATGTATGCATCTCTAG | | |
| | GAGAGAGAGTCACTATCACTTGCAAGGCGAGTCA | | |
| | GGACATTAATAGCTATTTAAGCTGGTTCCAGCAGA | | |
| | AACCAGGGAAATCTCCTAAGACCCTGATCTATCGT | | |
| | GCAAACAGATTGGTAGATGGGGTCCCATCAAGGT | | |
| | TCAGTGGCAGTGGATCTGGGCAAGATTATTCTCTC | | |
| | ACCATCAGCAGCCTGGAGTATGAAGATATGGGAA | | |
| | TTTATTATTGTCTACAGTATGATGAGTTTCCTCCGT | | |
| | GGACGTTCGGTGGAGGCACCAAGCTGGAAATCAAA | | |
| | Signal | ATGAGGACCCCTGCTCAGTTTCTTGGAAT | nt 1-60 of |
| | seq | CTTGTTGCTCTGGTTTCCAGGTATCAAAT | SEQ ID NO: 4 |
| | | GT | |
| | FR1 | GACATCAAGATGACCCAGTCTCCATCTTC | nt 61-129 of |
| | | CATGTATGCATCTCTAGGAGAGAGAGTC | SEQ ID NO: 4 |
| | | ACTATCACTTGC | |
| | CDR1 | AAGGCGAGTCAGGACATTAATAGCTATT | nt 130-162 of |
| | | TAAGC | SEQ ID NO: 4 |
| | FR2 | TGGTTCCAGCAGAAACCAGGGAAATCTC | nt 163-207 of |
| | | CTAAGACCCTGATCTAT | SEQ ID NO: 4 |
| | CDR2 | CGTGCAAACAGATTGGTAGAT | nt 208-228 of |
| | | | SEQ ID NO: 4 |
| | FR3 | GGGGTCCCATCAAGGTTCAGTGGCAGTG | nt 229-324 of |
| | | GATCTGGGCAAGATTATTCTCTCACCATC | SEQ ID NO: 4 |
| | | AGCAGCCTGGAGTATGAAGATATGGGAA | |
| | | TTTATTATTGT | |
| | CDR3 | CTACAGTATGATGAGTTTCCTCCGTGGACG | nt 325-354 of |
| | | | SEQ ID NO: 4 |
| | FR4 | TTCGGTGGAGGCACCAAGCTGGAAATCAAA | nt 355-384 of |
| | | | SEQ ID NO: 4 |
| amino | Signal sequence-FR1-CDR1-FR2-CDR2-FR3-CDR3-FR4 | | SEQ ID NO: 5 |
| acid | MRTPAQFLGILLLWFPGIKCDIKMTQSPSSMYASLGE | | |
| sequences | RVTITCKASQDINSYLSWFQQKPGKSPKTLIYRANRL | | |
| | VDGVPSRFSGSGSGQDYSLTISSLEYEDMGIYYCLQY | | |
| | DEFPPWTFGGGTKLEIK | | |
| | Signal | MRTPAQFLGILLLWFPGIKC | aa 1-20 of |
| | seq | | SEQ ID NO: 5 |
| | FR1 | DIKMTQSPSSMYASLGERVTTTC | aa 21-43 of |
| | | | SEQ ID NO: 5 |
| | CDR1 | KASQDINSYLS | aa 44-54 of |
| | | | SEQ ID NO: 5 |
| | FR2 | WFQQKPGKSPKTLIY | aa 55-69 of |
| | | | SEQ ID NO: 5 |
| | CDR2 | RANRLVD | aa 70-76 of |
| | | | SEQ ID NO: 5 |
| | FR3 | GVPSRFSGSGSGQDYSLTISSLEYEDMGIYYC | aa 77-108 of |
| | | | SEQ ID NO: 5 |
| | CDR3 | LQYDEFPPWT | aa 109-118 of |
| | | | SEQ ID NO: 5 |
| | FR4 | FGGGTKLEIK | aa 119-128 of |
| | | | SEQ ID NO: 5 |

9E12 heavy chain variable region
V-GENE and allele: Musmus IGHV1S127*01F
J-GENE and allele: Musmus IGHJ2*01F

| nucleic | Signal sequence-FR1 CDR1-FR2-CDR2-FR3-CDR3-FR4 | | SEQ ID NO: 6 |
|---|---|---|---|
| acid | ATGGGATGGAGCTGGATCTTTCTCTTTCTCCTGTCA | | |
| sequences | GGAACTGCAGGTGTCCTCTCTCAGGTCCAACTGCA | | |
| | GCAGCCTGGGGCTGAGCTGGTGAAGCCTGGGGCT | | |
| | TCAGTGAAGATGTCCTGCAAGGCTTCTGGCTACAC | | |
| | CTTCACCAGCTACTGGATGCACTGGGTGAAGCAGA | | |
| | GGCCTGGACAAGGCCTTGAGTGGATCGGAACGAT | | |
| | TGATCCTTCAGATAGTTATACTAGCTACAATCAAA | | |
| | AGTTCAAGGGCAAGGCCACATTGACTGTAGACAC | | |
| | ATCCTCCAGCACAGCCTACATGCAGCTCAGCAGCC | | |
| | TGACATCTGAGGACTCTGCGGTCTATTACTGTGAA | | |
| | GGGTTTGACTACTGGGGCCAAGGCACCACTCTCAC | | |
| | AGTCTCCTCA | | |
| | Signal | ATGGGATGGAGCTGGATCTTTCTCTTTCT | nt 1-57 of |
| | seq | CCTGTCAGGAACTGCAGGTGTCCTCTCT | SEQ ID NO: 6 |
| | FR1 | CAGGTCCAACTGCAGCAGCCTGGGGCTG | nt 58-147 of |
| | | AGCTGGTGAAGCCTGGGGCTTCAGTGAA | SEQ ID NO: 6 |
| | | GATGTCCTGCAAGGCTTCTGGCTACACCT | |
| | | TCACC | |
| | CDR1 | AGCTACTGGATGCAC | nt 148-162 of |
| | | | SEQ ID NO: 6 |
| | FR2 | TGGGTGAAGCAGAGGCCTGGACAAGGCC | nt 163-204 of |
| | | TTGAGTGGATCGGA | SEQ ID NO: 6 |
| | CDR2 | ACGATTGATCCTTCAGATAGTTATACTAG | nt 205-255 of |
| | | CTACAATCAAAAGTTCAAGGGC | SEQ ID NO: 6 |
| | FR3 | AAGGCCACATTGACTGTAGACACATCCT | nt 256-351 of |
| | | CCAGCACAGCCTACATGCAGCTCAGCAG | SEQ ID NO: 6 |
| | | CCTGACATCTGAGGACTCTGCGGTCTATT | |
| | | ACTGTGAAGGG | |
| | CDR3 | TTTGACTAC | nt 352-360 of |
| | | | SEQ ID NO: 6 |
| | FR4 | TGGGGCCAAGGCACCACTCTCACAGTCT | nt 361-393 of |
| | | CCTCA | SEQ ID NO: 6 |
| amino | Signal sequence-FR1 CDR1-FR2-CDR2-FR3-CDR3-FR4 | | SEQ ID NO: 7 |
| acid | MRWSCIILFLVATATGVNSQVQLQQPGAELVKPGAS | | |
| sequences | VKMSCKASGYTFTSYWMHWVKQRPGQGLEWIGTID | | |
| | PSDSYTSYNQKFKGKATLTVDTSSSTAYMQLSSLTSE | | |
| | DSAVYYCEGFDYWGQGTTLTVSS | | |
| | Signal | MRWSCIILFLVATATGVNS | aa 1-19 of |
| | seq | | SEQ ID NO: 7 |
| | FR1 | QVQLQQPGAELVKPGASVKMSCKASGYTFT | aa 20-49 of |
| | | | SEQ ID NO: 7 |
| | CDR1 | SWMH | aa 50-54 of |
| | | | SEQ ID NO: 7 |
| | FR2 | WVKQRPGQGLEWIG | aa 55-68 of |
| | | | SEQ ID NO: 7 |
| | CDR2 | TIDPSDSYTSYNQKFKG | aa 69-85 of |
| | | | SEQ ID NO: 7 |
| | FR3 | KATLTVDTSSSTAYMQLSSLTSEDSAVYYCEG | aa 86-117 of |
| | | | SEQ ID NO: 7 |
| | CDR3 | FDY | aa 118-120 of |
| | | | SEQ ID NO: 7 |
| | FR4 | WGQGTTLTVSS | aa 121-131 of |
| | | | SEQ ID NO: 7 |

9E12 light chain variable regions (IgM, Kappa)
V-GENE and allele: Musmus IGKV1-135*01 F
J-GENE and allele: Musmus IGKJ4*01 F

| nucleic | Signal sequence-FR1-CDR1-FR2-CDR2-FR3-CDR3-FR4 | | SEQ ID NO: 8 |
|---|---|---|---|
| acid | ATGAGTCCTGCCCAGTTCCTGTTTCTGTTAGTGCTC | | |
| sequences | TGGATTCGGGAAACCAACGGTGATGTTGTGATGAC | | |
| | CCAGACTCCACTCACTTTGTCGGTTACCATTGGAC | | |
| | AACCAGCCTCCATCTCTTGCAAGTCAAGTCAGAGC | | |
| | CTCTTAGATAGTGATGGAAAGACATATTTGAATTG | | |
| | GTTGTTACAGAGGCCAGGCCAGTCTCCAAAGCGCC | | |
| | TAATCTATCTGGTGTCTAAACTGGACTCTGGAGTC | | |
| | CCTGACAGGTTCACTGGCAGTGGATCAGGGACAG | | |
| | ATTTCACACTGAAAATCAGCAGAGTGGAGGCTGA | | |
| | GGATTTGGGAGTTTATTATTGCTGGCAAGGTACAC | | |
| | ATTTTCCATTCACGTTCGGCTCGGGGACAAAGTTG | | |
| | GAAATAAAA | | |
| | Signal | ATGAGTCCTGCCCAGTTCCTGTTTCTGTT | nt 1-57 of |
| | seq | AGTGCTCTGGATTCGGGAAACCAACGGT | SEQ ID NO: 8 |
| | FR1 | GATGTTGTGATGACCCAGACTCCACTCAC | nt 58-126 of |
| | | TTTGTCGGTTACCATTGGACAACCAGCCT | SEQ ID NO: 8 |
| | | CCATCTCTTGC | |
| | CDR1 | AAGTCAAGTCAGAGCCTCTTAGATAGTG | nt 127-174 of |
| | | ATGGAAAGACATATTTGAAT | SEQ ID NO: 8 |
| | FR2 | TGGTTGTTACAGAGGCCAGGCCAGTCTCC | nt 175-219 of |
| | | AAAGCGCCTAATCTAT | SEQ ID NO: 8 |
| | CDR2 | CTGGTGTCTAAACTGGACTCT | nt 220-240 of |
| | | | SEQ ID NO: 8 |
| | FR3 | GGAGTCCCTGACAGGTTCACTGGCAGTG | nt 241-336 of |
| | | GATCAGGGACAGATTTCACACTGAAAAT | SEQ ID NO: 8 |
| | | CAGCAGAGTGGAGGCTGAGGATTTGGGA | |
| | | GTTTATTATTGC | |
| | CDR3 | TGGCAAGGTACACATTTTCCATTCACG | nt 337-363 of |
| | | | SEQ ID NO: 8 |
| | FR4 | TTCGGCTCGGGGACAAAGTTGGAAATAA | nt 364-393 of |
| | | AA | SEQ ID NO: 8 |
| amino | Signal sequence-FR1-CDR1-FR2-CDR2-FR3-CDR3-FR4 | | SEQ ID NO: 9 |
| acid | MSPAQFLFLLVLWIRETNGDVVMTQTPLTLSVTIGQP | | |
| sequences | ASISCKSSQSLLDSDGKTYLNWLLQRPGQSPKRLIYL | | |
| | VSKLDSGVPDRFTGSGSGTDFTLKISRVEAEDLGVYY | | |
| | CWQGTHFPFTFGSGTKLEIK | | |
| | Signal | MSPAQFLFLLVLWIRETNG | aa 1-19 of |
| | seq | | SEQ ID NO: 9 |
| | FR1 | DVVMTQTPLTLSVTIGQPASISC | aa 20-42 of |
| | | | SEQ ID NO: 9 |
| | CDR1 | KSSQSLLDSDGKTYLN | aa 43-58 of |
| | | | SEQ ID NO: 9 |
| | FR2 | WLLQRPGQSPKRLIY | aa 59-73 of |
| | | | SEQ ID NO: 9 |
| | CDR2 | LVSKLDS | aa 74-80 of |
| | | | SEQ ID NO: 9 |
| | FR3 | GVPDRFTGSGSGTDFTLK1SRVEAEDLGVY | aa 81-112 of |
| | | YC | SEQ ID NO: 9 |
| | CDR3 | WQGTHFPFT | aa 113-121 of |
| | | | SEQ ID NO: 9 |
| | FR4 | FGSGTKLEIK | aa 122-131 of |
| | | | SEQ ID NO: 9 |

12D9 Heavy Cliain Variable Region
V-GENE and allele: Musmus IGHV9-3-1*01 F
J-GENE and allele: Musmus IGKJ4*01 F

| nucleic | Signal sequence-FR1-CDR1-FR2-CDR2-FR3-CDR3-FR4 | | SEQ ID NO: 10 |
|---|---|---|---|
| acid | ATGGCTTGGGTGTGGACCTTGCTATTCCTGATGGC | | |
| sequences | AGCTGCCCAAAGTGCCCAAGCACAGATCCAGTTG | | |
| | GTGCAGTCTGGACCTGAGCTGAAGAAGCCTGGAG | | |
| | AGACAGTCAAGATCTCCTGCAAGGCTTCTGGGTAT | | |
| | ACCTTCACAAACTATGGAATGAACTGGGTGAAGC | | |
| | AGACTCCAGGAAAGGGTTTAAAGTGGATGGGCTG | | |
| | GATAAACACCTACACTGGAGACtCCAACATATACT | | |
| | GATGACTTCAAGGGACGGTTTGCCTTCTCTTTGGA | | |
| | AACCTCTGCCAGCACTGCCTCTTTGCAGATCATCA | | |
| | ACCTCAAAAATGAGGACACGGCTACATATTTCTGT | | |
| | GCAAGGGGGGGCGATTTCTATGCTATGGACTACTG | | |
| | GGGTCAAGGAACCTCAGTCACCGTCTCCTCA | | |
| | Signal | ATGGCTTGGGTGTGGACCTTGCTATTCCT | nt 1-57 of |
| | seq | GATGGCAGCTGCCCAAAGTGCCCAAGCA | SEQ ID NO: 10 |
| | FR1 | CAGATCCAGTTGGTGCAGTCTGGACCTG | nt 58-147 of |
| | | AGCTGAAGAAGCCTGGAGAGACAGTCAA | SEQ ID NO: 10 |
| | | GATCTCCTGCAAGGCTTCTGGGTATACCT | |
| | | TCACA | |
| | CDR1 | AACTATGGAATGAAC | nt 148-162 of |
| | | | SEQ ID NO: 10 |
| | FR2 | TGGGTGAAGCAGACTCCAGGAAAGGGTT | nt 163-204 of |
| | | TAAAGTGGATGGGC | SEQ ID NO: 10 |
| | CDR2 | TGGATAAACACCTACACTGGAGAGCCAA | nt 205-255 of |
| | | CATATACTGATGACTTCAAGGGA | SEQ ID NO: 10 |
| | FR3 | CGGTTTGCCTTCTCTTTGGAAACCTCTGC | nt 256-351 of |
| | | CAGCACTGCCTCTTTGCAGATCATCAACC | SEQ ID NO: 10 |
| | | TCAAAAATGAGGACACGGCTACATATTT | |
| | | CTGTGCAAGG | |
| | CDR3 | GGGGGCGATTTCTATGCTATGGACTAC | nt 352-378 of |
| | | | SEQ ID NO: 10 |
| | FR4 | TGGGGTCAAGGAACCTCAGTCACCGTCT | nt 379-411 of |
| | | CCTCA | SEQ ID NO: 10 |
| amino | Signal sequence-FR1-CDR1-FR2-CDR2-FR3-CDR3-FR4 | | SEQ ID NO: 11 |
| acid | MAWVWTLLFLMAAAQSAQAQIQLVQSGPELKKPGE | | |
| sequences | TVKISCKASGYTFTNYGMNWVKQTPGKGLKWMGW | | |
| | INTYTGEPTYTDDFKGRFAFSLETSASTASLQIINLKN | | |
| | EDTATYFCARGGDFYAMDYWGQGTSVTVSS | | |
| | Signal | MAWVWTLLFLMAAAQSAQA | aa 1-19 of |
| | seq | | SEQ ID NO: 11 |
| | FR1 | QIQLVQSGPELKKPGETVK1SCKASGYTFT | aa 20-49 of |
| | | | SEQ ID NO: 11 |
| | CDR1 | NYGMN | aa 50-54 of |
| | | | SEQ ID NO: 11 |
| | FR2 | WVKQTPGKGLKWMG | aa 55-68 of |
| | | | SEQ ID NO: 11 |
| | CDR2 | WINTYTGEPTYTDDFKG | aa 69-85 of |
| | | | SEQ ID NO: 11 |
| | FR3 | RFAFSLETSASTASLQIINLKNEDTATYFCA | aa 86-117 of |
| | | R | SEQ ID NO: 11 |
| | CDR3 | GGDFYAMDY | aa 118-126 of |
| | | | SEQ ID NO: 11 |
| | FR4 | WGQGTSVTVSS | aa 127-137 of |
| | | | SEQ ID NO: 11 |

12D9-VL (IgM, Kappa)
V-GENE and allele: Musmus IGKV6-20*01 F
J-GENE and allele: Musmus IGKJ2*01 F

| nucleic | Signal sequence-FR1-CDR1-FR2-CDR2-FR3-CDR3-FR4 | | SEQ ID NO: 12 |
|---|---|---|---|
| acid | ATGGAATCACAGACTCTGGTCTTCATATCCATACT | | |
| sequences | GCTCTGGTTATATGGTGCTGATGGGAACATCGTAA | | |
| | TGACCCAATCTCCCAAATCCATGTCCATGTCAGTA | | |
| | GGAGAGAGGGTCACCTTGAGCTGCAAGGCCAGTG | | |
| | AGAATGTGGGTACTTATGTATCCTGGTATCAACAG | | |
| | AAACCAGAGCAGTCTCCTAAACTGGTGATATTCGG | | |
| | GGCATCCAACCGGAACACTGGGGTCCCCGATCGCT | | |
| | TCACAGGCAGTGGATTTGCAACAGATTTCACTCTG | | |
| | ACCATCAGCAGTGTGCAGGCTGAAGACCTTGGAG | | |
| | ATTATCACTGTGGACAGAGTTCCAACTATCCATAC | | |
| | ACGTTCGGAGGGGGGACCAAGCTGGAAATAAAA | | |
| | Signal | ATGGAATCACAGACTCTGGTCTTCATATC | nt 1-60 of |
| | seq | CATACTGCTCTGGTTATATGGTGCTGATG | SEQ ID NO: 12 |
| | | GG | |
| | FR1 | AACATCGIAATGACCCAATCTCCCAAATC | nt 61-129 of |
| | | CATGTCCATGTCAGTAGGAGAGAGGGTC | SEQ ID NO: 12 |
| | | ACCTTGAGCTGC | |
| | CDR1 | AAGGCCAGTGAGAATGTGGGTACTTATG | nt 130-162 of |
| | | TATCC | SEQ ID NO: 12 |
| | FR2 | TGGTATCAACAGAAACCAGAGCAGTCTC | nt 163-207 of |
| | | CTAAACTGGTGATATTC | SEQ ID NO: 12 |
| | CDR2 | GGGGCATCCAACCGGAACACT | nt 208-228 of |
| | | | SEQ ID NO: 12 |
| | FR3 | GGGGTCCCCGATCGCTTCACAGGCAGTG | nt 229-324 of |
| | | GATTTGCAACAGATTTCACTCTGACCATC | SEQ ID NO: 12 |
| | | AGCAGTGTGCAGGCTGAAGACCTTGGAG | |
| | | ATTATCACTGT | |
| | CDR3 | GGACAGAGTTCCAACTATCCATACACG | nt 325-351 of |
| | | | SEQ ID NO: 12 |
| | FR4 | TTCGGAGGGGGGACCAAGCTGGAAATAA | nt 352-381 of |
| | | AA | SEQ ID NO: 12 |
| amino | Signal sequence-FR1-CDR1-FR2-CDR2-FR3-CDR3-FR4 | | SEQIDNO:13 |
| acid | MESQTLVFISILLWLYGADGNIVMTQSPKSMSMSVG | | |
| sequences | ERVTLSCKASENVGTYVSWYQQKPEQSPKLVIFGAS | | |
| | NRNTGVPDRFTGSGFATDFTLTISSVQAEDLGDYHC | | |
| | GQSSNYPYTFGGGTKLEIK | | |
| | Signal | MESQTLVFISILLWLYGADG | aa 1-20 of |
| | seq | | SEQ ID NO: 13 |
| | FR1 | NIVMTQSPKSMSMSVGERVTLSC | aa 21-43 of |
| | | | SEQ ID NO: 13 |
| | CDR1 | KASENVGTYVS | aa 44-54 of |
| | | | SEQ ID NO: 13 |
| | FR2 | WYQQKPEQSPKLVIF | aa 55-69 of |
| | | | SEQ ID NO: 13 |
| | CDR2 | GASNRNT | aa 70-76 of |
| | | | SEQ ID NO: 13 |
| | FR3 | GVPDRFTGSGFATDFTLTISSVQAEDLGDY | aa 77-108 of |
| | | HC | SEQ ID NO: 13 |
| | CDR3 | GQSSNYPYT | aa 109-117 of |
| | | | SEQ ID NO: 13 |
| | FR4 | FGGGTKLEIK | aa 118-127 of |
| | | | SEQ ID NO: 13 |

12E4 Heavy Chain Variable Region
V-GENE and allele: Musmus IGHV1-18*01 F, or Musmus IGHV1-26*01 F
J-GENE and ailele: Musmus IGHJ4*01 F

| nucleic | Signal sequence-FR1-CDR1-FR2-CDR2-FR3-CDR3-FR4 | | SEQ ID NO: 14 |
|---|---|---|---|
| acid | ATGGGATGGAGCTGGATCTTTCTCTTCCTCCTGTC | | |
| sequences | AGGAACTGCAGGTGTCCACTCTGAGGTCCAGCTGC | | |
| | AACAGTCTGGACCTGAGCTGGTGAAGCCTGGAGC | | |
| | TTCAATGAAGATATCCTGCAAGGCTTCTGGTTACT | | |
| | CATTCACTGGCTACACCATGAACTGGGTGAAGCAG | | |
| | AGCCATGGAAAGAACCTTGAGTGGATTGGACTTAT | | |
| | TAATCCTTACAATGGTGGTACTAGCTACAACCAGA | | |
| | AGTTCAAGGGCAAGGCCACATTAACTGTAGACAA | | |
| | GTCATCCAGCACAGCCTACATGGAGCTCCTCAGTC | | |
| | TGACATCTGAGGACTCTGCAGTCTATTACTGTGCA | | |
| | AGAGGGGGCTTCTATGCTATGGACTACTGGGGTCA | | |
| | AGGAACCTCAGTCACCGTCTCCTCA | | |
| | Signal | ATGGGATGGAGCTGGATCTTTCTCTTCCT | nt I-57 of |
| | seq | CCTGTCAGGAACTGCAGGTGTCCACTCT | SEQ ID NO: 14 |
| | FR1 | GAGGTCCAGCTGCAACAGTCTGGACCTG | nt 58-147 of |
| | | AGCTGGTGAAGCCTGGAGCTTCAATGAA | SEQ ID NO: 14 |
| | | GATATCCTGCAAGGCTTCTGGTTACTCAT | |
| | | TCACT | |
| | CDR1 | GGCTACACCATGAAC | nt 148-162 of |
| | | | SEQ ID NO: 14 |
| | FR2 | TGGGTGAAGCAGAGCCATGGAAAGAACC | nt 163-204 of |
| | | TTGAGTGGATTGGA | SEQ ID NO: 14 |
| | CDR2 | CTTATTAATCCTTACAATGGTGGTACTAG | nt 205-255 of |
| | | CTACAACCAGAAGTTCAAGGGC | SEQ ID NO: 14 |
| | FR3 | AAGGCCACATTAACTGTAGACAAGTCAT | ni256-351 of |
| | | CCAGCACAGCCTACATGGAGCTCCTCAG | SEQ ID NO: 14 |
| | | TCTGACATCTGAGGACTCTGCAGTCTATT | |
| | | ACTGTGCAAGA | |
| | CDR3 | GGGGGCTTCTATGCTATGGACTAC | nt 352-375 of |
| | | | SEQ ID NO: 14 |
| | FR4 | TGGGGTCAAGGAACCTCAGTCACCGTCT | nt 376-408 of |
| | | CCTCA | SEQ ID NO: 14 |
| amino | Signal sequence-FR1-CDR1-FR2-CDR2-FR3-CDR3-FR4 | | SEQ ID NO: 15 |
| acid | MGWSWIFLFLLSGTAGVHSEVQLQQSGPELVKPGAS | | |
| sequences | MKISCKASGYSFTGYTMNWVKQSHGKNLEWIGLINP | | |
| | YNGGTSYNQKFKGKATLTVDKSSSTAYMELLSLTSE | | |
| | DSAVYYCARGGFYAMDYWGQGTSVTVSS | | |
| | Signal | MGWSWIFLFLLSGTAGVHS | aa 1-19 of |
| | seq | | SEQ ID NO: 15 |
| | FR1 | EVQLQQSGPELVKPGASMK1SCKASGYSFT | aa 20-49 of |
| | | | SEQ ID NO: 15 |
| | CDR1 | GYTMN | aa 50-54 of |
| | | | SEQ ID NO: 15 |
| | FR2 | WVKQSHGKNLEWIG | aa 55-68 of |
| | | | SEQ ID NO: 15 |
| | CDR2 | LINPYNGGTSYNQKFKG | aa 69-85 of |
| | | | SEQ ID NO: 15 |
| | FR3 | KATLTVDKSSSTAYMELLSLTSEDSAVYYC | aa 86-117 of |
| | | AR | SEQ ID NO: 15 |
| | CDR3 | GGFYAMDY | aa 118-125 of |
| | | | SEQ ID NO: 15 |
| | FR4 | WGQGTSVTVSS | aa 126-136 of |
| | | | SEQ ID NO: 15 |

12E4 Light Chain Variable Region (IgM Kappa)
V-GENE and allele: Musmus IGKV 10-94*01 F
J-GENE and allele: Musmus IGKJ2*01 F

| nucleic | Signal sequence-FRl CDR1-FR2-CDR2-FR3-CDR3-FR4 | | SEQ ID NO: 16 |
|---|---|---|---|
| acid | ATGTCCTCTGCTCAGTTCCTTGGTCTCCTGTTGCTC | | |
| sequences | TGTTTTCAAGGTACCAGATGTGATATCCAGATGAC | | |
| | ACAGACTACATCCTCCCTGTCTGCCTCTCTGGGAG | | |
| | ACAGAGTCACCATCAGTTGCAGTGCAAGTCAGGG | | |
| | CATTAGCAATTATTTAAACTGGTATCAGCAGAAAC | | |
| | CAGATGGAACTGTTAAACTCCTGATCTATTACACA | | |
| | TCAAGTTTACACTCAGGAGTCCCATCAAGGTTCAG | | |
| | TGGCAGTGGGTCTGGGACAGATTATTCTCTCACCA | | |
| | TCAGCAACCTGGAACCTGAAGATATTGCCACTTAC | | |
| | TATTGTCAGCAGTATAGTAAGCTTCCGTACACGTT | | |
| | CGGAGGGGGGACCAAGCTGGAAATAAAA | | |
| | Signal | ATGTCCTCTGCTCAGTTCCTTGGTCTCCT | nt 1-57 of |
| | seq | GTTGCTCTGTTTTCAAGGTACCAGATGT | SEQ ID NO: 16 |
| | FR1 | GATATCCAGATGACACAGACTACATCCT | nt 58-126 of |
| | | CCCTGTCTGCCTCTCTGGGAGACAGAGTC | SEQ ID NO: 16 |
| | | ACCATCAGTTGC | |
| | CDR1 | AGTGCAAGTCAGGGCATTAGCAATTATTT | nt 127-159 of |
| | | AAAC | SEQ ID NO: 16 |
| | FR2 | TGGTATCAGCAGAAACCAGATGGAACTG | nt 160-204 of |
| | | TTAAACTCCTGATCTAT | SEQ ID NO: 16 |
| | CDR2 | TACACATCAAGTTTACACTCA | nt 205-225 of |
| | | | SEQ ID NO: 16 |
| | FR3 | GGAGTCCCATCAAGGTTCAGTGGCAGTG | nt 226-321 of |
| | | GGTCTGGGACAGATTATTCTCTCACCATC | SEQ ID NO: 16 |
| | | AGCAACCTGGAACCTGAAGATATTGCCA | |
| | | CTTACTATTGT | |
| | CDR3 | CAGCAGTATAGTAAGCTTCCGTACACG | nt 322-348 of |
| | | | SEQ ID NO: 16 |
| | FR4 | TTCGGAGGGGGGACCAAGCTGGAAATAA | nt 349-378 of |
| | | AA | SEQ ID NO: 16 |
| amino | Signal sequence-FR1-CDR1-FR2-CDR2-FR3-CDR3-FR4 | | SEQ ID NO: 17 |
| acid | MSSAQFLGLLLLCFQGTRCDIQMTQTTSSLSASLGDR | | |
| sequences | VTISCSASQGISNYLNWYQQKPDGTVKLLIYYTSSLH | | |
| | SGVPSRFSGSGSGTDYSLTISNLEPEDIATYYCQQYSK | | |
| | LPYTFGGGTKLEIK | | |
| | Signal | MSSAQFLGLLLLCFQGTRC | aa 1-19 of |
| | seq | | SEQ ID NO: 17 |
| | FR1 | DIQMTQTTSSLSASLGDRVTISC | aa 20-42 of |
| | | | SEQ ID NO: 17 |
| | CDR1 | SASQGISNYLN | aa 43-53 of |
| | | | SEQ ID NO: 17 |
| | FR2 | WYQQKPDGTYKLLIY | aa 54-68 of |
| | | | SEQ ID NO: 17 |
| | CDR2 | YTSSLHS | aa 69-75 of |
| | | | SEQ ID NO: 17 |
| | FR3 | GVPSRFSGSGSGTDYSLTISNLEPEDIATYY | aa 76-107 of |
| | | C | SEQ ID NO: 17 |
| | CDR3 | QQYSKLPYT | aa 108-116 of |
| | | | SEQ ID NO: 17 |
| | FR4 | FGGGTKLEIK | aa 117-126 of |
| | | | SEQ ID NO: 17 |

4D6 Heavy Chain Variable Region
V-GENE and allele: Musmus IGHV1-9*01 F
J-GENE and allele: Musmus IGHJ3*01 F

| nucleic | Signal sequence-FR1-CDR1-FR2-CDR2-FR3-CDR3-FR4 | | SEQ ID NO: 18 |
|---|---|---|---|
| acid | ATGGAATGGACCTGGGTCTTTCTCTTCCTCCTGTCA | | |
| sequences | GTAACTGCAGGTGTCCGCTCCCAGGTTCAGCTGCA | | |
| | GCAGTCTGGAGCTGAGCTGATGAAGCCTGGGGCC | | |
| | TCAGTGAAGATATCCTGCAAGGCTACTGGCTACAC | | |
| | ATTCAGTAGCTACTGGATAGAGTGGGTAAAGCAG | | |
| | AGGCCTGGACATGGCCTTGAGTGGATTGGAGAGA | | |
| | TTTTACCTGGAAGTGATAGTACTTACTACAATGAG | | |
| | AACTTCAAGGGCAAGGCCACATTCACTGCAGATA | | |
| | CATCCTCCAACACAGCCTACATACAACTCAGCAGC | | |
| | CTGACATCTGAGGACTCTGCCGTCTATTACTGTGC | | |
| | AAGTGACAGCTCGGGCTTGTTTGCTTACTGGGGCC | | |
| | AAGGGACTCTGGTCACTGTCTCTGCA | | |
| | Signal | ATGGAATGGACCTGGGTCTTTCTCTTCCT | nt 1-57 of |
| | seq | CCTGTCAGTAACTGCAGGTGTCCGCTCC | SEQ ID NO: 18 |
| | FR1 | CAGGTTCAGCTGCAGCAGTCTGGAGCTG | nt 58-147 of |
| | | AGCTGATGAAGCCTGGGGCCTCAGTGAA | SEQ ID NO: 18 |
| | | GATATCCTGCAAGGCTACTGGCTACACAT | |
| | | TCAGT | |
| | CDR1 | AGCTACTGGATAGAG | nt 148-162 of |
| | | | SEQ ID NO: 18 |
| | FR2 | TGGGTAAAGCAGAGGCCTGGACATGGCC | nt 163-204 of |
| | | TTGAGTGGATTGGA | SEQ ID NO: 18 |
| | CDR2 | GAGATTTTACCTGGAAGTGATAGTACTTA | nt 205-255 of |
| | | CTACAATGAGAACTTCAAGGGC | SEQ ID NO: 18 |
| | FR3 | AAGGCCACATTCACTGCAGATACATCCTC | nt 256-351 of |
| | | CAACACAGCCTACATACAACTCAGCAGC | SEQ ID NO: 38 |
| | | CTGACATCTGAGGACTCTGCCGTCTATTA | |
| | | CTGTGCAAGT | |
| | CDR3 | GACAGCTCGGGCTTGTTTGCTTAC | nt 352-375 of |
| | | | SEQ ID NO: 18 |
| | FR4 | TGGGGCCAAGGGACTCTGGTCACTGTCTC | nt 376-408 of |
| | | TGCA | SEQ ID NO: 18 |
| amino | Signal sequence-FR1-CDR1-FR2-CDR2-FR3-CDR3-FR4 | | SEQ ID NO: 19 |
| acid | MEWTVVVFLFLLSVTAGVRSQVQLQQSGAELMKPGA | | |
| sequences | SVKISCKATGYTFSSYWIEWVKQRPGMGLEWTGEILP | | |
| | GSDSTYYNENFKGKATFTADTSSNTAYIQLSSLTSED | | |
| | SAVYYCASDSSGLFAYWGQGTLVTVSA | | |
| | Signal | MEWTWVFLFLLSVTAGVRS | aa 1-19 of |
| | seq | | SEQ ID NO: 19 |
| | FR1 | QVQLQQSGAELMKPGASVKISCKATGYTF | aa 20-49 of |
| | | S | SEQ ID NO: 19 |
| | CDR1 | SYWIE | aa 50-54 of |
| | | | SEQ ID NO: 19 |
| | FR2 | WVKQRPGHGLEWIG | aa 55-68 of |
| | | | SEQ ID NO: 19 |
| | CDR2 | EILPGSDSTYYNENFKG | aa 69-85 of |
| | | | SEQ ID NO: 19 |
| | FR3 | KATFTADTSSNTAYIQLSSLTSEDSAVYYC | aa 86-117 of |
| | | AS | SEQ ID NO: 19 |
| | CDR3 | DSSGLFAY | aa 118-125 of |
| | | | SEQ ID NO: 19 |
| | FR4 | WGQGTLVTVSA | aa 126-136 of |
| | | | SEQ ID NO: 19 |

4D6 clone 1 Light Chain Variable Region (IgM, Kappa)
V-GENE and allele: Musmus IGKV6-32*01 F
J-GENE and allele: Musmus IGKJ5*01 F

| nucleic | Signal sequence-FR1-CDR1-FR2-CDR2-FR3-CDR3-FR4 | | SEQ ID NO: 20 |
|---|---|---|---|
| acid | ATGAAGTCACAGACCCAGGTCTTCGTATTTCTACT | | |
| sequences | CiCTCTGTGTGTCTGGTGCTCATGGGAGTATTGTGA | | |
| | TGACCCAGACTCCCAAATTCCTGCTTGTATCAGCA | | |
| | GGAGACAGGGTTACCATAACCTGCAAGGCCAGTC | | |
| | AGAGTGTGAGTAATGATGTAGCTTGGTACCAACA | | |
| | GAAGCCAGGGCAGTCTCCTAAACTGCTGATATACT | | |
| | ATGCATCCAATCGCTACACTGGAGTCCCTGATCGC | | |
| | TTCACTGGCAGTGGATATGGGACGGATTTCACTTT | | |
| | CACCATCAGCACTGTGCAGGCTGAAGACCTGGCA | | |
| | GTTTATTTCTGTCAGCAGGATTATAGCTCTCCGCTC | | |
| | ACGTTCGGTGCTGGGACCAAGCTGGAGCTGAAA | | |
| | Signal | ATGAAGTCACAGACCCAGGTCTTCGTATT | nt 1-60 of |
| | seq | TCTACTGCTCTGTGTGTCTGGTGCTCATG | SEQ ID NO: 20 |
| | | GG | |
| | FR1 | AGTATTGTGATGACCCAGACTCCCAAATT | nt 61-129 of |
| | | CCTGCTTGTATCAGCAGGAGACAGGGTT | SEQ ID NO: 20 |
| | | ACCATAACCTGC | |
| | CDR1 | AAGGCCAGTCAGAGTGTGAGTAATGATG | nt 130-162 of |
| | | TAGCT | SEQ ID NO: 20 |
| | FR2 | TGGTACCAACAGAAGCCAGGGCAGTCTC | nt 163-207 of |
| | | CTAAACTGCTGATATAC | SEQ ID NO: 20 |
| | CDR2 | TATGCATCCAATCGCTACACT | nt 208-228 of |
| | | | SEQ ID NO: 20 |
| | FR3 | GGAGTCCCTGATCGCTTCACTGGCAGTGG | nt 229-324 of |
| | | ATATGGGACGGATTTCACTTTCACCATCA | SEQ ID NO: 20 |
| | | GCACTGTGCAGGCTGAAGACCTGGCAGT | |
| | | TTATTTCTGT | |
| | CDR3 | CAGCAGGATTATAGCTCTCCGCTCACG | nt 325-351 of |
| | | | SEQ ID NO: 20 |
| | FR4 | TTCGGTGCTGGGACCAAGCTGGAGCTGA | nt 352-381 of |
| | | AA | SEQ ID NO: 20 |
| amino | Signal sequence-FR1 CDR1-FR2-CDR2-FR3-CDR3-FR4 | | SEQ ID NO: 21 |
| acid | MKSQTQVFVFLLLCVSGAHGSIVMTQTPKFLLVSAG | | |
| sequences | DRVTITCKASQSVSNDVAWYQQKPGQSPKLLIYYAS | | |
| | NRYTGVPDRFTGSGYGTDFTFTISTVQAEDLAVYFC | | |
| | QQDYSSPLTFGAGTKLELK | | |
| | Signal | MKSQTQVFVFLLLCVSGAHG | aa I-20 of |
| | seq | | SEQ ID NO: 21 |
| | FR1 | S1VMTQTPKFLLVSAGDRVTITC | aa 21-43 of |
| | | | SEQ ID NO: 21 |
| | CDR1 | KASQSVSNDVA | aa 44-54 of |
| | | | SEQ ID NO: 21 |
| | FR2 | WYQQKPGQSPKLLIY | aa 55-69 of |
| | | | SEQ ID NO: 21 |
| | CDR2 | YASNRYT | aa 70-76 of |
| | | | SEQ ID NO: 21 |
| | FR3 | GVPDRFTGSGYGTDFTFTISTVQAEDLAVY | aa 77-108 of |
| | | FC | SEQ ID NO: 21 |
| | CDR3 | QQDYSSPLT | aa 109-117 of |
| | | | SEQ ID NO: 21 |
| | FR4 | FGAGTKLELK | aa 118-127 of |
| | | | SEQ ID NO: 21 |

4D6-VL (clone 2) Light Chain Variable Region (IgM, Kappa)
V-GENE and allele: Musmus IGKV3-9*01 F
J-GENE and allele: Musmus IGKJ4*01 F

| nucleic | Signal sequence-FR1-CDR1-FR2-CDR2-FR3-CDR3-FR4 | | SEQ ID NO: 22 |
|---|---|---|---|
| acid | ATGGAGACAGACCCACTCCTGCTATGGGTGCTGCT | | |
| sequences | GCTCTGGGTTCCAGGCTCCACTGGTGACATTGTGC | | |
| | TGACCCAATCTCCAGCTTCTTTGGCTGTGTCTCTAG | | |
| | GACAGAGGGCCACCATATCCTGCCAAGCCAGCGA | | |
| | AAGTGTCAGTTTTGCTGATACAAGTTTAATGCACT | | |
| | GGTACCAACAGAAACCAGGACAGCCACCCAAACT | | |
| | CCTCATCTATCGTGCATCCAACCTAGAATCTGGAG | | |
| | TCCCTGCCAGGTTCACTGGCAGTGGGTCTGAGTCA | | |
| | GACTTCACTCTCACCATCGATCCTGTGGAGGAAGA | | |
| | TGATGCTGCAATGTATTACTGTATGCAAAGTATGG | | |
| | AAGATCCATTCACGTTCGGCTCGGGGACAAAGTTG | | |
| | GAAATAAAA | | |
| | Signal | ATGGAGACAGACCCACTCCTGCTATGGG | nt 1-60 of |
| | seq | TGCTGCTGCTCTGGGTTCCAGGCTCCACT | SEQ ID NO: 22 |
| | | GGT | |
| | FR1 | GACATTGTGCTGACCCAATCTCCAGCTTC | nt 61-129 of |
| | | TTTGGCTGTGTCTCTAGGACAGAGGGCCA | SEQ ID NO: 22 |
| | | CCATATCCTGC | |
| | CDR1 | CAAGCCAGCGAAAGTGTCAGTTTTGCTG | nt 130-174 of |
| | | ATACAAGTTTAATGCAC | SEQ ID NO: 22 |
| | FR2 | TGGTACCAACAGAAACCAGGACAGCCAC | nt 175-219 of |
| | | CCAAACTCCTCATCTAT | SEQ ID NO: 22 |
| | CDR2 | CGTGCATCCAACCTAGAATCT | nt 220-240 of |
| | | | SEQ ID NO: 22 |
| | FR3 | GGAGTCCCTGCCAGGTTCACTGGCAGTG | nt 241-336 of |
| | | GGTCTGAGTCAGACTTCACTCTCACCATC | SEQ ID NO: 22 |
| | | GATCCTGTGGAGGAAGATGATGCTGCAA | |
| | | TGTATTACTGT | |
| | CDR3 | ATGCAAAGTATGGAAGATCCATTCACG | nt 337-363 of |
| | | | SEQ ID NO: 22 |
| | FR4 | TTCGGCTCGGGGACAAAGTTGGAAATAA | nt 364-393 of |
| | | AA | SEQ ID NO: 22 |
| amino | Signal sequence-FR1-CDR1-FR2-CDR2-FR3-CDR3-FR4 | | SEQ ID NO: 23 |
| acid | METDPLLLWVLLLWVPGSTGDIVLTQSPASLAVSLG | | |
| sequences | QRATISCQASESVSFADTSLMHWYQQKPGQPPKLLI | | |
| | YRASNLESGVPARFTGSGSESDFTLTIDPVEEDDAAM | | |
| | YYCMQSMEDPFTFGSGTKLEIK | | |
| | Signal | METDPLLLWVLLLWVPGSTG | aa 1-20 of |
| | seq | | SEQ ID NO: 23 |
| | FR1 | DIVLTQSPASLAVSLGQRATISC | aa 21-43 of |
| | | | SEQ ID NO: 23 |
| | CDR1 | QASESVSFADTSLMH | aa 44-58 of |
| | | | SEQ ID NO: 23 |
| | FR2 | WYQQKPGQPPKLLIY | aa 59-73 of |
| | | | SEQ ID NO: 23 |
| | CDR2 | RASNLES | aa 74-80 of |
| | | | SEQ ID NO: 23 |
| | FR3 | GVPARFTGSGSESDFTLTIDPVEEDDAAMY | aa 81-112 of |
| | | YC | SEQ ID NO: 23 |
| | CDR3 | MQSMEDPFT | aa 113-121 of |
| | | | SEQ ID NO: 23 |
| | FR4 | FGSGTKLEIK | aa 122-131 of |
| | | | SEQ ID NO: 23 |

3D8 Heavy Chain Variable Region
V-GENE and allele: Musmus IGHV1-14HHF
J-GENE and allele: Musmus IGHJ2*01F

| nucleic | Signal sequence-FR1-CDR1-FR2-CDR2-FR3-CDR3-FR4 | | SEQ ID NO: 24 |
|---|---|---|---|
| acid | ATGGAATGGAGTTGGATATTTCTCTTTCTCCTGTCA | | |
| sequences | GGAACTGCAGGTGTCCACTCTGAGGTCCAGCTGCA | | |
| | GCAGTCTGGACCTGAGCTGGTAAAGCCTGGGGCTT | | |
| | CAGTGAAGATGTCCTGCAAGGCTTCTGGATACACA | | |
| | TTCACTAGCTATGTTATGCACTGGGTGAAGCAGAA | | |
| | GCCTGGGCAGGGCCTTGAGTGGATTGGATATATTA | | |
| | ATCCTTACAATGATGGTACTAAGTACAATGAGAAG | | |
| | TTCAAAGGCAAGGCCACACTGACTTCAGACAAAT | | |
| | CCTCCAGCACAGCCTACATGGAGCTCAGCAGCCTG | | |
| | ACCTCTGAGGACTCTGCGGTCTATTACTGTGCAAG | | |
| | GGGGGTCTATGATGGTTACCCTGACTACTGGGGCC | | |
| | AAGGCACCACTCTCACAGTCTCCTCA | | |
| | Signal | ATGGAATGGAGTTGGATATTTCTCTTTCT | nt 1-57 of |
| | seq | CCTGTCAGGAACTGCAGGTGTCCACTCT | SEQ ID NO: 24 |
| | FR1 | GAGGTCCAGCTGCAGCAGTCTGGACCTG | nt 58-147 of |
| | | AGCTGGTAAAGCCTGGGGCTTCAGTGAA | SEQ ID NO: 24 |
| | | GATGTCCTGCAAGGCTTCTGGATACACAT | |
| | | TCACT | |
| | CDR1 | AGCTATGTTATGCAC | nt 148-162 of |
| | | | SEQ ID NO: 24 |
| | FR2 | TGGGTGAAGCAGAAGCCTGGGCAGGGCC | nt 163-204 of |
| | | TTGAGTGGATTGGA | SEQ ID NO: 24 |
| | CDR2 | TATATTAATCCTTACAATGATGGTACTAA | nt 205-255 of |
| | | GTACAATGAGAAGTTCAAAGGC | SEQ ID NO: 24 |
| | FR3 | AAGGCCACACTGACTTCAGACAAATCCT | nt 256-351 of |
| | | CCAGCACAGCCTACATGGAGCTCAGCAG | SEQ ID NO: 24 |
| | | CCTGACCTCTGAGGACTCTGCGGTCTATT | |
| | | ACTGTGCAAGG | |
| | CDR3 | GGGGTCTATGATGGTTACCCTGACTAC | nt 352-378 of |
| | | | SEQ ID NO: 24 |
| | FR4 | TGGGGCCAAGGCACCACTCTCACAGTCT | nt 379-411 of |
| | | CCTCA | SEQ ID NO: 24 |
| amino | Signal sequence-FR1-CDR1-FR2-CDR2-FR3-CDR3-FR4 | | SEQ ID NO: 25 |
| acid | MEWSWIFLFLLSGTAGVHSEVQLQQSGPELVKPGAS | | |
| sequences | VKMSCKASGYTFTSYVMHWVKQKPGQGLEWIGYIN | | |
| | PYNDGTKYNEKFKGKATLTSDKSSSTAYMELSSLTS | | |
| | EDSAVYYCARGVYDGYPDYWGQGTTLTVSS | | |
| | Signal | MEWSWIFLFLLSGTAGVHS | aa 1-19 of |
| | seq | | SEQ ID NO: 25 |
| | FR1 | EVQLQQSGPELVKPGASVKMSCKASGYTF | aa 20-49 of |
| | | T | SEQ ID NO: 25 |
| | CDR1 | SYVMH | aa 50-54 of |
| | | | SEQ ID NO: 25 |
| | FR2 | WVKQKPGQGLEWIG | aa 55-68 of |
| | | | SEQ ID NO: 25 |
| | CDR2 | YINPYNDGTKYNEKFKG | aa 69-85 of |
| | | | SEQ ID NO: 25 |
| | FR3 | KATLTSDKSSSTAYMELSSLTSEDSAVYYC | aa 86-117 of |
| | | AR | SEQ ID NO: 25 |
| | CDR3 | GVYDGYPDY | aa 118-126 of |
| | | | SEQ ID NO: 25 |
| | FR4 | WGQGTTLTVSS | aa 127-137 of |
| | | | SEQ ID NO: 25 |

3D8 Light Chain Variable Region (IgM, Kappa)
V-GENE and allele: Musmus IGKV5-39*01F
J-GENE and allele: Musmus IGKJ2*01 F

| nucleic | Signal sequence-FR1-CDR1-FR2-CDR2-FR3-CDR3-FR4 | | SEQ ID NO: 26 |
|---|---|---|---|
| acid | ATGGTGTCCACTTCTCAGCTCCTTGGACTTTTGCTT | | |
| sequences | TTCTGGACTTCAGCCTCCAGATGTGACATTGTGAT | | |
| | GACTCAGTCTCCAGCCACCCTGTCTGTGACTCCAG | | |
| | GAGATAGAGTCTCTCTTTCCTGCAGGGCCAGCCAG | | |
| | AGTATTAGCGACTACTTACACTGGTATCAACAAAA | | |
| | ATCACATGAGTCTCCAAGGCTTCTCATCAAATATG | | |
| | CTTCCCAATCCATCTCTGGGATCCCCTCCAGGTTC | | |
| | AGTGGCAGTGGATCAGGGTCAGATTTCACTCTCAG | | |
| | TATCAACAGTGTGGAACCTGAAGATGTTGGAGTGT | | |
| | ATTACTGTCAAAATGGTCACAGCTTTCCGTACACG | | |
| | TTCGGAGGGGGGACCAAGCTGGAAATAAAA | | |
| | Signal | ATGGTGTCCACTTCTCAGCTCCTTGGACT | nt 1-60 of |
| | seq | TTTGCTTTTCTGGACTTCAGCCTCCAGAT | SEQ ID NO: 26 |
| | | GT | |
| | FR1 | GACATTGTGATGACTCAGTCTCCAGCCAC | nt 61-129 of |
| | | CCTGTCTGT GACTCCAGGAGATAGAGTCT | SEQ ID NO: 26 |
| | | CTCTTTCCTGC | |
| | CDR1 | AGGGCCAGCCAGAGTATTAGCGACTACT | nt 130-162 of |
| | | TACAC | SEQ ID NO: 26 |
| | FR2 | TGGTATCAACAAAAATCACATGAGTCTC | nt 163-207 of |
| | | CAAGGCTTCTCATCAAA | SEQ ID NO: 26 |
| | CDR2 | TATGCTTCCCAATCCATCTCT | nt 208-228 of |
| | | | SEQ ID NO: 26 |
| | FR3 | GGGATCCCCTCCAGGTTCAGTGGCAGTG | nt 229-324 of |
| | | GATCAGGGTCAGATTTCACTCTCAGTATC | SEQ ID NO: 26 |
| | | AACAGTGTGGAACCTGAAGATGTTGGAG | |
| | | TGTATTACTGT | |
| | CDR3 | CAAAATGGTCACAGCTTTCCGTACACG | nt 325-351 of |
| | | | SEQ ID NO: 26 |
| | FR4 | TTCGGAGGGGGGACCAAGCTGGAAATAA | nt 352-381 of |
| | | AA | SEQ ID NO: 26 |
| amino | Signal sequence-FR3 CDR1-FR2-CDR2-FR3-CDR3-FR4 | | SEQ ID NO: 27 |
| acid | MVSTSQLLGLLLFWTSASRCDIVMTQSPATLSVTPG | | |
| sequences | DRVSLSCRASQSISDYLHWYQQKSHESPRLLIKYASQ | | |
| | S1SGIPSRFSGSGSGSDFTLS1NSVEPEDVGVYYCQNG | | |
| | HSFPYTFGGGTKLEIK | | |
| | Signal | MVSTSQLLGLLLFWTSASRC | aa 1-20 of |
| | seq | | SEQ ID NO: 27 |
| | FR1 | DIVMTQSPATLSVTPGDRVSLSC | aa 21-43 of |
| | | | SEQ ID NO: 27 |
| | CDR1 | RASQSISDYLH | aa 44-54 of |
| | | | SEQ ID NO: 27 |
| | FR2 | WYQQKSHESPRLLIK | aa 55-69 of |
| | | | SEQ ID NO: 27 |
| | CDR2 | YASQSIS | aa 70-76 of |
| | | | SEQ ID NO: 27 |
| | FR3 | GIPSRFSGSGSGSDFTLSINSVEPEDVGVYY | aa 77-108 of |
| | | C | SEQ ID NO: 27 |
| | CDR3 | QNGHSFPYT | Aa109-117 of |
| | | | SEQ ID NO: 27 |
| | FR4 | FGGGTKLEIK | aa 118-127 of |
| | | | SEQ ID NO: 27 |

In certain embodiments, a CDR identified as an amino acid sequence in Tables 1-5 may be truncated with 1, 2, 3 or 4 amino acids in the N terminal and/or the C terminal. In certain embodiments, a CDR may be a CDR identified as an amino acid sequence in Tables 1-5 but shifted to the N terminal side or the C terminal side by 1, 2, 3 or 4 amino acids. In certain embodiments, a CDR may be a CDR identified as an amino acid sequence in Tables 1-5 but extended to the N terminal side or the C terminal side by 1, 2, 3 or 4 amino acids. In certain embodiments, a CDR is any combination of the CDRs as described in this paragraph.

In certain embodiments, a CDR identified as a nucleic acid sequence in Tables 1-5 may be truncated with 1, 2, 3, 4, 5, 6, 7, 8, 9, 10, 11, or 12 nucleotides in the 5′ end and/or the 3′ end. In certain embodiments, a CDR may be a CDR identified as nucleic acid sequence in Tables 1-5 shifted to the 5′ and/or the 3′ side by 1, 2, 3, 4, 5, 6, 7, 8, 9, 10, 11, or 12 nucleotides, in certain embodiments, a CDR may be a CDR identified as nucleic acid sequence in Tables 1-5 but extended to the 5′ and/or the 3′ side by 1, 2, 3, 4, 5, 6, 7, 8, 9, 10, 11, or 12 nucleotides. In certain embodiments, a CDR is any combination of the CDRs as described in this paragraph.

In certain embodiments, provided herein is an antibody, or a variant thereof, or an epitope binding fragment thereof comprising any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or all 33 CDR(s) as described. In certain embodiments, the antibody, or a variant thereof, or an epitope binding fragment thereof comprises one or more copies of the CDR(s).

As used herein, the complementarity-determining region (CDR) refers to part of the variable chains in antibodies or T cell receptors, which binds to the corresponding epitope. Such CDR may be determined via experiments or via various predicating tools, such as www.imgt.org/IMGT_vquest/analysis. Also provided herein is a nucleic acid sequence encoding an antibody, or a variant thereof, or an epitope binding fragment thereof as described herein. As used herein, an epitope binding fragment refers to a fragment of an antibody which is determined to be bound to an epitope. Such determination may be performed experimentally using for example ELISA or other methods discussed herein or via various predicating tools such as IMGT.org.

In certain embodiments, the antibody or fragment thereof comprising at least one of: a heavy chain variable region (VII) encoded by a V_{H} coding sequence of 3D8, 6A10, 9E12, 12D9, 12E4 or 4D6 or a nucleic acid sequence at least 85% identical thereto; or a light chain variable region (V_{L}) encoded by a V_{H} coding sequence of 3D8, 6A10, 9E12, 12D9, 12E4, 4D6 clone 1 or 4D6 clone 2 or a nucleic acid sequence at least 85% identical thereto; or a heavy chain variable region having a V_{H} amino acid sequence of 3D8, 6A10, 9E12, 12D9, 12E4 or 4D6 or an amino acid sequence at least 85% identical thereto; or a light chain variable region having a V_{L} amino acid sequence of 3D8, 6A10, 9E12, 12D9, 12E4, 4D6 clone 1 or 4D6 clone 2 or an amino acid sequence at least 85% identical thereto.

In certain embodiments, V_{H} or V_{L} coding sequences or amino acid sequences are provided in Table 5 as well as in SEQ ID NOs: 2-27. In certain embodiments, a V_{H} or V_{L} identified as an amino acid sequence in Table 5 as well as in SEQ ID NOs: 2-27 may be truncated with 1, 2, 3, 4, 5, 6, 7, 8, 9, 10 or more amino acids in the N terminal and/or the C terminal. In certain embodiments, a V_{H} or V_{L} may be a V_{H} or V_{L} identified as an amino acid sequence in Table 5 as well as in SEQ ID NOs: 2-27 but shifted to the N terminal side or the C terminal side by 1, 2, 3, 4, 5, 6, 7, 8, 9, 10 or more amino acids. In certain embodiments, a V_{H} or V_{L}, is any combination of the V_{H} or V_{L} s as described in this paragraph.

In certain embodiments, a V_{H} or V_{L}, identified as a nucleic acid sequence in in Table 5 as well as in SEQ ID NOs: 2-27 may be truncated with 1, 2, 3, 4, 5, 6, 7, 8, 9, 10, 11, 12, 15, 18, 21, 24, 27, 30, 33, 36, 39 or more nucleotides in the 5′ end and/or the 3′ end. In certain embodiments, a V_{H} or V_{L} may be a V_{H} or V_{L} identified as nucleic acid sequence in Table 5 as well as in SEQ ID NOs: 2-27 but, shifted to the 5′ and/or the 3′ side by 1, 2, 3, 4, 5, 6, 7, 8, 9, 10, 11, 12, 15, 18, 21, 24, 27, 30, 33, 36, 39 or more nucleotides (nt). In certain embodiments, a V_{H} or V_{L} is any combination of the V_{H} or V_{L} as described in this paragraph.

In certain embodiments, provided herein is an antibody, or a variant thereof, or an epitope binding fragment thereof comprising any 1, 2, 3, 4, 5, 6, 7, 8, 9, 10, or all 11 V_{H} or V_{L} as described. In certain embodiments, the antibody, or a variant thereof, or an epitope binding fragment thereof comprises one or more copies of the V_{H} or V_{L}.

In certain embodiments, the antibody or fragment thereof comprising at least one of: a heavy chain variable region encoded by a nucleic acid sequence of SEQ ID NOs: 2, 6, 10, 14, 18, or 24 or a nucleic acid sequence at least 85% identical thereto; or a light chain variable region encoded by a nucleic acid sequence of SEQ ID NOs: 4, 8, 12, 16, 20, 22 or 26 or a nucleic acid sequence at least 85% identical thereto; or a heavy chain variable region having an amino acid sequence of SEQ ID NOs: 3, 7, 11, 15, 19 or 25 or an amino acid sequence at least 85% identical thereto; or a light chain variable region having an amino acid sequence of SEQ ID NOs: 5, 9, 13, 17, 21, 23 or 27 or an amino acid sequence at least 85% identical thereto.

In certain embodiments, the antibody or epitope binding fragment thereof comprises a heavy chain variable region encoded by a nucleic acid sequence of SEQ ID NO: 2 or a nucleic acid sequence at least 85% identical thereto. Additionally, or alternatively, the antibody or epitope binding fragment thereof comprises a light chain variable region encoded by a nucleic acid sequence of SEQ ID NO: 4 or a nucleic acid sequence at least 85% identical thereto. In certain embodiments, the antibody or epitope binding fragment thereof comprises a heavy chain variable region having an amino acid sequence of SEQ ID NO: 3 or an amino acid sequence at least 85% identical thereto. Additionally, or alternatively, the antibody or epitope binding fragment thereof comprises a light chain variable region having an amino acid sequence of SEQ ID NO: 5 or an amino acid sequence at least 85% identical thereto.

In certain embodiments, the antibody or epitope binding fragment thereof comprises a heavy chain variable region encoded by a nucleic acid sequence of SEQ ID NO: 6 or a nucleic acid sequence at least 85% identical thereto, Additionally, or alternatively, the antibody or epitope binding fragment thereof comprises a light chain variable region encoded by a nucleic acid sequence of SEQ ID NO: 8 or a nucleic acid sequence at least 85% identical thereto. In certain embodiments, the antibody or epitope binding fragment thereof comprises a heavy chain variable region having an amino acid sequence of SEQ ID NO: 7 or an amino acid sequence at least 85% identical thereto. Additionally, or alternatively, the antibody or epitope binding fragment thereof comprises a light chain variable region having an amino acid sequence of SEQ ID NO: 9 or an amino acid sequence at least 85% identical thereto.

In certain embodiments, the antibody or epitope binding fragment thereof comprises a heavy chain variable region encoded by a nucleic acid sequence of SEQ ID NO: 10 or a nucleic acid sequence at least 85% identical thereto. Additionally, or alternatively, the antibody or epitope binding fragment thereof comprises a light chain variable region encoded by a nucleic acid sequence of SEQ ID NO: 12 or a nucleic acid sequence at least 85% identical thereto. In certain embodiments, the antibody or epitope binding fragment thereof comprises a heavy chain variable region having an amino acid sequence of SEQ ID NO: 11 or an amino acid sequence at least 85% identical thereto. Additionally, or alternatively, the antibody or epitope binding fragment thereof comprises a light chain variable region having an amino acid sequence f SEQ ID NO: 13 or an amino acid sequence at least 85% identical thereto.

In certain embodiments, the antibody or epitope binding fragment thereof comprises a heavy chain variable region encoded by a nucleic acid sequence of SEQ ID NO: 14 or a nucleic acid sequence at least 85% identical thereto. Additionally, or alternatively, the antibody or epitope binding fragment thereof comprises a light chain variable region encoded by a nucleic acid sequence of SEQ ID NO: 16 or a nucleic acid sequence at least 85% identical thereto. In certain embodiments, the antibody or epitope binding fragment thereof comprises a heavy chain variable region having an amino acid sequence of SEQ ID NO: 15 or an amino acid sequence at least 85% identical thereto. Additionally, or alternatively, the antibody or epi tope binding fragment thereof comprises a light chain variable region having an amino acid sequence of SEQ ID NO: 17 or an amino acid sequence at least 85% identical thereto.

In certain embodiments, the antibody or epitope binding fragment thereof comprises a heavy chain variable region encoded by a nucleic acid sequence of SEQ ID NO: 18 or a nucleic acid sequence at least 85% identical thereto. Additionally, or alternatively, the antibody or epitope binding fragment thereof comprises a light chain variable region encoded by a nucleic acid sequence of SEQ ID NO: 20 or a nucleic acid sequence at least 85% identical thereto. In certain embodiments, the antibody or epitope binding fragment thereof comprises a heavy chain variable region having an amino acid sequence of SEQ ID NO: 19 or an amino acid sequence at least 85% identical thereto. Additionally, or alternatively, the antibody or epitope binding fragment thereof comprises a light chain variable region having an amino acid sequence of SEQ ID NO: 21 or an amino acid sequence at least 85% identical thereto.

In certain embodiments, the antibody or epitope binding fragment thereof comprises a heavy chain variable region encoded by a nucleic acid sequence of SEQ ID NO: 18 or a nucleic acid sequence at least 85% identical thereto. Additionally, or alternatively, the antibody or epitope binding fragment thereof comprises a light chain variable region encoded by a nucleic acid sequence of SEQ ID NO: 22 or a nucleic acid sequence at least 85% identical thereto. In certain embodiments, the antibody or epitope binding fragment thereof comprises a heavy chain variable region having an amino acid sequence of SEQ ID NO: 19 or an amino acid sequence at least 85% identical thereto. Additionally, or alternatively, the antibody or epitope binding fragment thereof comprises a light chain variable region having an amino acid sequence of SEQ ID NO: 23 or an amino acid sequence at least 85% identical thereto.

In certain embodiments, the antibody or epitope binding fragment thereof comprises a heavy chain variable region encoded by a nucleic acid sequence of SEQ ID NO: 24 or a nucleic acid sequence at least 85% identical thereto. Additionally, or alternatively, the antibody or epitope binding fragment thereof comprises a light chain variable region encoded by a nucleic acid sequence of SEQ ID NO: 26 or a nucleic acid sequence at least 85% identical thereto. In certain embodiments, the antibody or epitope binding fragment thereof comprises a heavy chain variable region having an amino acid sequence of SEQ ID NO: 25 or an amino acid sequence at least 85% identical thereto. Additionally, or alternatively, the antibody or epitope binding fragment thereof comprises a light chain variable region having an amino acid sequence of SEQ ID NO: 27 or an amino acid sequence at least 85% identical thereto.

Utilizing any of the nucleotide sequences encoding the heavy chain variable region of 6A10 (SEQ ID NO: 2), 9E12 (SEQ ID NO: 6), 12D9 (SEQ ID NO: 10), 12E4 (SEQ ID NO: 14), 4D6 (SEQ ID NO: 18), and 3D8 (SEQ ID NO: 24); the nucleotide sequences encoding the light chain variable region of 6A10 (SEQ ID NO: 4), 9E12 (SEQ ID NO: 8), 12D9 (SEQ ID NO: 12), 12E4 (SEQ ID NO: 16), 4D6 clone 1 (SEQ ID NO: 20), 4D6 clone 2 (SEQ ID NO: 22), and 3D8 (SEQ ID NO: 26), their encoded amino acid sequences for the heavy chain variable region of 6A10 (SEQ ID NO: 3), 9E12 (SEQ ID NO: 7), 12D9 (SEQ ID NO: 11), 12E4 (SEQ ID NO: 15), 4D6 (SEQ ID NO: 19), and 3D8 (SEQ ID NO: 25); and their encoded amino acid sequences for the light chain variable region of 6A10 (SEQ ID NO: 5), 9E12 (SEQ ID NO: 9), 12D9 (SEQ ID NO: 13), 12E4 (SEQ ID NO: 17), 4D6 clone 1 (SEQ ID NO: 21), 4D6 clone 2 (SEQ ID NO: 23), and 3D8 (SEQ ID NO: 27), or nucleotide or amino acid sequences sharing at least about 80% (for example, about 80%, about 85%, about 90%, about 95%, about 96%, about 97%, about 98%, or about 99%), at least about 85%, at least about 90%, at least about 95%, at least about 96%, at least about 97%, at least about 98%, at least about 99%, or at least about 99.9% sequence identity therewith, other antibodies or fragments that specifically bind to a LOX-1 epitope, can be generated.

In certain embodiments, the antibody or a epitope binding fragment thereof comprises an amino acid sequence sharing a certain percentage (which is less than 100%) identity to any one of SEQ ID NOs: 3, 5, 7, 9, 11, 13, 15, 17, 19, 21, 23, 25, and 27 and is different from the sequence with the corresponding SEQ ID NO in the region other than CDR(s). In certain embodiments, the antibody or a epitope binding fragment thereof comprises an amino acid sequence sharing a certain percentage (which is less than 100%) identity to any one of SEQ ID NOs: 3, 5, 7, 9, 11, 13, 15, 17, 19, 21, 23, 25, and 27 and is able to bind to LOX-1 epitope at an affinity of at least 10%, 20%, 30%, 40%, 50%, 60%, 70%, 80%, 90%, 95%, 100%, 1.5 fold, 2 fold, 3 fold, 4 fold, 5 fold, 10 fold of the affinity of 6A10, or 9E12, or 12D9, or 12E4, or 4D6, or 3D8. As used herein, affinity of an antibody or peptide binding fragment thereof refers to the strength with which an epitope binds to the antibody or peptide binding fragment thereof. Such strength may be measured as described in the Example.

The availability of these nucleic acid molecules encoding the heavy and light chains of the antibody enables production of a recombinant, antibody, fragment or modifications using in vitro expression methods and cell-free expression systems known in the art. In vitro transcription and translation systems are commercially available, e.g., from Promega Biotech (Madison, Wis.) or Gibco-BRL (Gaithersburg, Md.). The antibodies, epitope-binding fragments or modifications thereof may also be produced by expression in a suitable prokaryotic or eukaryotic system. Similarly, modifications may be inserted into these sequences by use of a variety of CRISPR techniques and other related, zinc finger, methodologies for modifying amino acid and nucleotide sequences.

These monoclonal antibodies 3D8, 6A10, 9E12, 12D9, 12E4, and 4D6 and their variable chain sequences identified herein can be further used to prepare other forms of antibodies, e.g., chimeric antibodies, humanized antibodies, human antibodies. Other antibody fragments or ligands can be produced by screening phage display libraries, antibody fragments and mixtures thereof. Techniques for generating these types of antibodies and ligands are well-known in the art and the ligands themselves may be generated using the disclosed amino acid sequences of the above-identified monoclonal antibodies.

In certain embodiments, the antibody or epitope binding fragment thereof is an IgG or comprises an IgG backbone. In certain embodiments, the antibody or epitope binding fragment is an intact IgA, IgG, IgM, IgE, IgD, IgG1, IgG2, IgG3 or IgG4, or a fragment thereof, or comprises a backbone thereof.

In certain embodiments, the antibody or epitope binding fragment thereof is a humanized antibody, a mouse antibody, a rabbit antibody, a goat antibody, a donkey antibody, a camelid antibody, or a fragment thereof.

Chimeric antibodies may similarly be developed using known techniques. Chimeric antibodies are molecules in which different portions are derived from different animal species. Single chain antibodies may also be prepared by conventional methods, such as described in U.S. Pat. Nos. 4,946,778 and 4,704,692 using the variable portions of the polyclonal or monoclonal antibodies produced according to this invention. Antibody fragments, such as the Fab, F(ab)₂ and scFv fragments and libraries thereof may also be employed in generation of the selective anti-LOX-1 antibodies as described herein.

In certain embodiments, the antibody or epitope binding fragment thereof is a bi-specific antibody, a monoclonal antibody, a chimeric antibody, a humanized antibody, a human antibody, a CDR-grafted antibody, a multispecific binding construct that can bind two or more targets, a dual specific antibody, a hi-specific antibody or a multi-specific antibody, or an affinity matured antibody, a single-domain antibody (sdAb), a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)₂ construct, a monovalent or bivalent construct from which domains non-essential to monoclonal antibody function have been removed, a single-chain molecule containing one V_{L}, one V_{H} antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or a fragment thereof.

In certain embodiments, the antibodies and epitope binding fragment thereof may be further modified from those exemplified. For example, the antibodies may be humanized. In a particular embodiment, the selected sequences of the heavy or light chains of any of the antibodies disclosed herein (or a portion thereof) are inserted into the backbone of an antibody or antibody fragment construct. For example, the variable light domain and/or variable heavy domain of the antibodies described herein may be inserted into another antibody construct. In certain embodiments, the heavy and light chains/variable regions may be connected with a peptide linker. Still other antibody modifications employing the SEQ ID NOs disclosed herein, e.g., as taught by the techniques referenced in above-cited. U.S. Pat. No. 9,902,772, incorporated by reference herein.

In certain embodiments, the antibody or epitope binding fragment thereof is hi-specific and comprises CDRs specifically recognizing and binding to more than one (for example, about 2, 3, 4, 5 or more) LOX-1 epitope. In certain embodiments, the antibody or epitope binding fragment thereof is hi-specific and comprises CDRs specifically recognizing and binding to a non-LOX-1 epitope, for example, an epitope of a biomarker for PMN-MDSC including CD15, CD66b, CD11b, CD33, or CD14. The production of hi-specific antibodies or ligands that specifically bind to two or more selected epitopes, can employ conventional techniques. See, e.g., Hornig N, Farber-Schwarz A., Production of bispecific antibodies: diabodies and tandem scFv, 2012, Methods Mol Biol., 907:713-27; Speiss, C. et al, Bispecific antibodies with natural architecture produced by co-culture of bacteria expressing two distinct half-antibodies, Jul. 7, 2013, Nature Biotechnology, 31:753-758; and Jonathan S Martin and Zhenping Zhu, Recombinant approaches to IgG-like bispecific antibodies, 2005 Acta Pharmacologica Sinica, 26: 649-658.

In certain embodiments, the antibody or epitope binding fragment thereof is coupled covalently or non-covalently (or conjugated to) to a detectable label. In certain embodiments, the detectable label is an enzyme, a fluorescent label, a radioisotope, or a chemiluminescent label. In certain embodiments, the detectable label is a His tag.

In certain embodiments, the antibody or epitope binding fragment thereof is an antibody-drug conjugate. In certain embodiments, the antibody or epitope binding fragment thereof is covalently or non-covalently attached to or associated with (or coupled to, or conjugated to) a cytotoxic agent, for example, a chemotherapy drug or a radioactive particle. In certain embodiments, the antibody or epitope binding fragment thereof recognizes and binds a LOX-1 epitope expressed on a cell surface of a target cell and the coupled cytotoxic agent induces cell death of the target cell. In certain embodiments, the target cell is a LOX-1 expressing neutrophil. In certain embodiments, the target cell is a LOX-1 expressing PMN. In certain embodiments, the target cell is a PMN-MDSC. In certain embodiments, the target, cell is a LOX-1 positive PMN-MDSC.

In certain embodiments, the antibody or epitope binding fragment thereof may be immobilized on a substrate, for example, a plate, a bead or a slide.

### Compositions

A variety of compositions are provided, such as the ones useful in producing the antibody or epitope binding fragment thereof. For example, a hybridoma cell or a host cell producing the described antibody or an epitope binding fragment thereof. In certain embodiments, the hybridoma cell or host cell comprises a hLOX-1 coding sequence as described herein.

Also provided are compositions useful in detecting expression of hLOX-1 protein, or in identifying, detecting and optionally separating cells (for example, PBMC, neutrophil or PMN) expressing hLOX-1 (for example on cell surface or in a cell) (i.e. LOX-1 positive cells) from cells not expressing hLOX-1 (i.e. LOX-1 negative cells) and other debris.

Additionally, provided are compositions useful in diagnosing presence, progression or metastasis of a cancer in a subject. In one embodiment, a diagnostic reagent composition or a kit is provided comprising one or more of the antibodies or epi tope binding fragments thereof as described herein.

In a further embodiment, the diagnostic composition or the kit may further comprise a ligand that binds other biomarker/genetic signatures of the PMN-MDSCs, such as those listed in Table 1 in US Patent Application Publication No. US20180059115, and/or a biomarker or a regulator of pathways for ER stress response, such as IRE-1 RNase, sXBP1, DDIT3 (CHOP), ATF4, ATF3, SEC61A ARGI, NOS-2, MYCN, CSF3, IL3, TGFβ1, LDL, RAF1, APP, IL6 PDGFBB, EPO, CD40LG, NFkB, IL13, AGT, IL1β, ERBB2, MAP2K1, VEGFα, CSF1, FLI1, or IFNγ. Still other likely biomarkers for pathways involved or activated in PMN-MDSC production may be included. In one embodiment, the ligand may be covalently or non-covalently joined with a detectable label or substrate. Selection and/or generation of suitable ligands with optional labels for use in this invention is within the skill of the art, provided with this specification, the documents incorporated herein, and the conventional teachings of the art. In a further embodiment, the diagnostic reagent composition or the kit also contains miscellaneous reagents and apparatus for reading labels, e.g., certain substrates that interact with an enzymatic label to produce a color signal, etc., apparatus for taking blood samples, as well as appropriate vials and other diagnostic assay components.

Further provided are pharmaceutical compositions useful in reducing, inhibiting, retaining, or suppressing growth of the PMN-MDSC population, and/or useful in treating a cancer. For example, provided is a composition comprising the antibody or epitope binding fragment thereof as described herein and a pharmaceutically acceptable carrier and/or excipient. In certain embodiments, the pharmaceutical composition reduces or inhibits ER stress in mammalian neutrophils or reduces or inhibits LOX-1 expression on neutrophil populations. In one embodiment, this composition further comprises an antagonist or inhibitor of the expression, activity or activation of one or more of IRE-1 RNase, sXBP1, DDIT3 (CHOP), ATF4, ATF3, SEC61A ARGI or NOS-2. In certain embodiments, this composition comprises an ER stress antagonist B-I09. In one embodiment, the composition further comprises an antagonist or inhibitor of LOX-1. In still further embodiments, the composition contains additional antagonist, or inhibitor of the expression, activity or activation of one or more of IRE-1 RNase, MYCN, CSF3, IL3, TGFβ1, TNF, LDL, RAF1, APP, IL6 PDGFBB, EPO, CD40LG, NFkB, IL13, AGT, IL1β, ERBB2, MAP2K1, VEGFα; CSF1 FLI1, or IFNγ; or of the pathways leading to the production of the immunosuppressive PMN-MDSC populations in vivo. In certain embodiments, the composition further comprises a chemotherapy drug for treating cancer. In certain embodiments, the composition is formulated with another effective compound or reagent for treatment of the cancers described herein, such as an antibiotic or bactericide, a surfactant, or other reagent commonly used in formulation of anti-cancer compositions.

The forms of the pharmaceutical compositions may be liquid, solid or a suspension or semi-solid and designed for use with a desired administrative route, such as those described herein. The doses and dosage regimens are adjusted for the particular cancer, and the stage of the cancer, physical status of the subject. Such doses may range from about 1 to about 100 mg/kg subject body weight and include dosage regimens designed to administer the effective amount in smaller repeated doses.

### Methods

In one aspect, methods for treatment of a cancer associated with LOX+1 or another disease or disorder associated with LOX+1 utilize the compositions and kits as described herein. In another aspect, these described compositions and kits are useful for monitoring differentiation and/or population of polymorphonuclear myeloid derived suppressor cells (PMN-MDSCs), for identifying a LOX-1 inhibitor and/or a PMN-MDSC inhibitor, and/or for cancer diagnosis, and diagnosis of other LOX-1+-related diseases or disorders.

### A. Diagnostic Methods

In one aspect, a method is provided for monitoring the population of polymorphonuclear myeloid derived suppressor cells (PMN-MDSCs) in a subject comprising contacting a biological sample from the subject containing polymorphonuclear neutrophils (PMNs) and PMN-MDSC with one or more of the antibody or epitope binding fragment described herein or the diagnostic composition; and detecting and optionally distinguishing LOX-1 positive (LOX-1⁺) cells from LOX-1 negative (LOX-1⁻) cells in the sample, wherein the LOX-1⁺ cells are PMN-MDSCs substantially five of PMN. In one embodiment, the method may be used for diagnosing a cancer, a cancer progression or metastasis in a subject. In one embodiment, the subject is diagnosed with a cancer if percentage of LOX-1⁺ cells in the total neutrophils in the sample is greater than a control.

In one aspect, provided is a method for monitoring population of polymorphonuclear myeloid derived suppressor cells (PMN-MDSCs) in a subject comprising obtaining a biological sample containing polymorphonuclear neutrophils (PMNs) and PMN-MDSC; contacting the biological sample with one or more of the antibody or epi tope binding fragment thereof or the diagnostic composition as described herein; and detecting and optionally distinguishing LOX-1 positive (LOX-1⁺) cells from LOX-1 negative (LOX-1⁻) cells in the sample, wherein the LOX-1+ cells are PMN-MDSCs substantially free of PMN.

In certain embodiments, LOX-1 positive cells refer to cells expressing LOX-1 protein, for example, on cell surface or in the cell. In certain embodiments, LOX-1 negative cells refer to cells which does not express LOX-1 protein or does not express LOX-1 protein on their cell surface.

Also provided is a method of diagnosing a cancer or monitoring progression or metastasis of a cancer in a subject comprising obtaining a biological sample from the subject; contacting the sample with one or more of the antibody or epitope binding fragment thereof or the diagnostic composition as described herein; and detecting and optionally distinguishing LOX-1 positive cells from LOX-1 negative cells in the sample. In certain embodiments, the subject is diagnosed with cancer or cancer progression (for example, tumor size) in the subject by correlation with the concentration of LOX-1⁺ cells detected. In certain embodiments, the subject is diagnosed with cancer or cancer metastasis if number of the LOX-1 positive cells is above that of a negative control. In certain embodiments, cancer progression (for example, tumor size) of the subject is determined by counting the LOX-1 positive cells and comparing it to a control.

In certain embodiments, cells of the methods are PBMC, neutrophil, or PMN. As used herein, in certain embodiments, PMN refers to cells showing biomarkers shared by PMN-MDSC and PMN which is not PMN-MDSC, i.e., PMN-MDSC is a subgroup of PMN. In certain embodiments, PMN only refers to the one that is not PMN-MDSC and/or is not LOX-1 positive.

In certain embodiments of the methods, the subject is suspected of having a cancer, had a cancer, is having a cancer, is suspected of having a cancer progression, or is suspected of having a cancer metastasis.

In some of the methods described herein, a control level is used as a reference point. The control level can be any of those described herein. In one embodiment, the control level is the level obtained from an individual, or a population of individuals, who are healthy (i.e., who do not have a cancer). In another embodiment, the control level is the level obtained from an individual, or a population of individuals, who have cancer that has not metastasized. In yet another embodiment, the control level is the level obtained from an individual, or a population of individuals, who have cancer at different progression stages (for example, having different tumor sizes).

In certain embodiments, the method further comprises counting or collecting the LOX-1+ cells, or LOX-1+ neutrophils, or LOX-1+ PMNs. In a further embodiment, the method comprises counting the total number of the cells, or neutrophils, or PMNs and calculation rations of LOX-1+ cells/neutrophils/PMNs.

In certain embodiments, the method comprises washing to reduce or eliminate LOX-1 negative cells and other debris in the sample. In certain embodiments, the method comprises separating LOX-1+ cells from LOX-1− cells or neutrophils from non-neutrophil cells in the sample based on cell size. In certain embodiments, the method further comprises destroying or lysing any red blood cells in the sample to permit their elimination from the sample and possible interference with the results of the assay. In certain embodiments, the method further comprises digesting a biological sample (such as a tissue) and releasing cells from the sample. Exemplary lytic reagents, stabilizing reagents and the method of use have been described, e.g., in U.S. Pat. Nos. 6,573,102 and 6,869,798. Alternatively, the reagent system can also be an isotonic lysing reagent as described in U.S. Pat. No. 5,882,934. Other lytic reagents known in the art can also be used for the purpose of the present methods.

In certain embodiments, the method further comprises collecting LOX-1 negative cells which contains PMNs and being substantially free from PMN-MDSCs. As used herein, A “being substantially free” from B refers to a mixture of A and B, wherein the ratio of number of A to that of B is at least about 5:1, or about 10:1, or about 20:1, or about 50:1, about 100:1, about 200:1, about 500:1, about 1000:1 or more. In one embodiment, the ratio of number of A to that of B is about 10:1.

In certain embodiments, a population of LOX-1 positive cells greater than a control level, or about 1%, about 2%, about 3%, about. 4%, about 5%, about 10%, about 15%, or about 20% of the total neutrophil population in the sample of a subject indicates the presence, progression or metastasis of a cancer. In certain embodiments, the population of LOX-1 positive cells greater than about 5% of the total neutrophil population in the sample of a subject indicates presence, progression, or metastasis of a cancer.

In one embodiment, provided is an assay method for diagnosis of having a cancer in a subject comprising: contacting a biological sample obtained from a patient with one or more of the antibody or epitope binding fragment thereof as described herein; detecting the level of binding between a component of said biological sample and the antibody or epitope binding fragment thereof; and diagnosing the subject with a cancer if a population of LOX-1⁺ cells greater than a control level or 1% of the total neutrophil population in the sample of a subject. In another embodiment, these methods can diagnose the aggressiveness of a cancer. In another embodiment, these methods can diagnose the stage of a cancer. According to the inventors' early studies, in most healthy individuals the proportion of LOX-1+ PMN is less than between 0.5% to 1% PMN. Patients with stage II diseases usually have between about 3 about 5% of LOX-1+ PMN and patients at stages III-IV have over 5% to about 12% PMN.

In certain embodiments, the method described herein may further comprise contacting one or more of the antibody or epitope binding fragment thereof as described herein with a LOX-1 expressing cell, serving as a positive control. In certain embodiments, patients with a cancer/tumor, a cancer/tumor metastasis or at a specific progression stage are served as a positive control. In certain embodiments, the method may further comprise contacting a biological sample from a subject who does not have cancer, or a biological sample pooled from subjects who do not have cancer with one or more of the antibody or epitope binding fragment thereof as described herein, serving as a negative control. In certain embodiments, the control level is acquired from a pooled sample or is an average of more than one control levels.

In certain embodiments, a patient is diagnosed as having a cancer, a cancer progression, or cancer metastasis, if amount/concentration of LOX-1+ cells/neutrophils/PMNs or LOX-1 expression level on the LOX-1+ cells/neutrophils/PMNs is greater than a negative control. Alternatively or additionally, the amount/concentration of LOX-1+ cells/neutrophils/PMNs or the LOX-1 expression level on the LOX-1+ cells/neutrophils/PMNs is at least about 10%, about 20%, about 30%, about 40%, about 50%, about 60%, about 70%, about 80%, about 90%, about 100%, about 1 fold, about 2 fold, about 5 fold, or about 10 fold of that of a positive control.

In certain embodiments, detection of amount/concentration of LOX-1+ cells/neutrophils/PMNs in a biological sample or LOX-1 expression level on the LOX-1+ cells/neutrophils/PMNs may be performed by detecting level of the detectable label. In a further embodiment, the method further comprises diagnosing the patient as having a cancer, cancer progression or cancer metastasis when the detectable label is detected at a level greater than that in a negative control. Alternatively or additionally, the method further comprises diagnosing the patient as having a cancer, cancer progression or cancer metastasis when the detectable label is detected at a level which is at least about 10%, about 20%, about 30%, about. 40%, about 50%, about 60%, about 70%, about 80%, about 90%, about 100%, about 1 fold, about 2 fold, about 5 fold, or about 10 fold of that of a positive control.

A method for enhancing or inhibiting differentiating polymorphonuclear myeloid derived suppressor cells (PMN-MDSCs) from polymorphonuclear neutrophils (PMNs) or monocytic myeloid derived suppressor cells (M-MDSCs) in a biological sample containing these types of cells involves the following steps. The biological sample, e.g., whole blood or a cell suspension, or a tumor exudate, or tissue, e.g., biopsy material, is contacted with an antibody or epitope binding fragments thereof. Thus, one may detect antibody-conjugate complexes in the sample. Such detection can be based upon separation of the ligand-bound cells from unbound cells in the sample. The LOX-1-bound cells are PMN-MDSCs substantially free of PMN.

The detection and separation of LOX-1 positive cells in the sample may be accomplished by a physical characteristic, such as the difference in size or weight of the LOX-1 positive cells vs. the LOX-1 negative cells. Such detection and/or separation techniques can thus employ appropriately sized filtration units, or the use of flow cytometry, or chromatographic or centrifugation techniques (size exclusion or weight exclusion), among others known to the art.

The method of identifying and separating PMN-MDSCs from a sample can also include contacting the biological sample with other biomarkers that identity as a single population both PMN-MDSCs and PMNs and/or M-MDSCs and isolating a cell suspension containing PMN-MDSCs and PMNs (and/or M-MDSCs) prior to, or simultaneously with, contacting the cell suspension with the LOX-1 antibody or epitope binding fragment thereof. In still other embodiments of the methods, the sample may be contacted (with or without RBC lysis) with a LOX-1 antibody or epitope binding fragment thereof and a ligand that identifies neutrophils, i.e., other PMN that are not LOX-1⁺. In one embodiment, the sample is contacted with a LOX-1 ligand and a CD15 ligand. In still other embodiments of the methods, the sample may be contacted with a LOX-1 antibody or epitope binding fragment thereof and a CD66b ligand. Still other ligands that identify neutrophils generally may be useful in this context.

In certain embodiments, the contacting step further comprises contacting the sample with a ligand that specifically binds to or forms a complex with a neutrophil biomarker to identify neutrophil or polymorphonuclear neutrophils (PMNs). In one embodiment, the neutrophil biomarker is CD33, CD11b, CD14, CD15 or CD66b. In one embodiment, the detecting step comprises detecting and optionally distinguishing LOX-1 positive neutrophils from LOX-1 negative cells in the sample. In a further embodiment, the diagnosing step comprises diagnosing the subject with cancer or determining cancer progression (for example, tumor size) in the subject by correlation with the concentration of LOX-1+ neutrophils detected.

In certain embodiments, the contacting step further comprises contacting the sample with a ligand that specifically binds to or forms a complex with a polymorphonuclear neutrophils (PMNs) biomarker to identify PMNs. In one embodiment, the biomarker may be any one or more of CD33, CD11b, CD 14, CD15 or CD66b. In one embodiment, the method further comprises a step of identifying cells with biomarkers shared by both PMN-MDSCs and PMNs, and isolating cells of both PMN-MDSCs and PMNs prior to the contacting step. In one embodiment, the detecting step comprises detecting and optionally distinguishing LOX-1 positive PMNs from LOX-1 negative cells in the sample. In a further embodiment, the diagnosing step comprises diagnosing the subject with cancer or determining cancer progression (for example, tumor size) in the subject by correlation with the concentration of LOX-1+ PMNs detected.

The method of identifying and separating PMN-MDSCs from a sample can also include contacting the biological sample with the other biomarkers forming the distinguishing signature of PMN-MDSC or other biomarkers that identify as a single population both PMN-MDSCs and PMNs and/or M-MDSCs and isolating a cell suspension containing PMN-MDSCs and PMNs (and/or M-MDSCs) prior to, or simultaneously with, contacting the cell suspension with the LOX-1 antibody or epitope binding fragment thereof. In still other embodiments of the methods, the sample may be contacted (with or without RBC lysis) with a LOX-1 antibody or epitope binding fragment thereof and a ligand that identifies neutrophils, i.e., other PMN that are not LOX-1⁺. In one embodiment, the sample is contacted with a LOX-1 ligand and a CD15 ligand. In still other embodiments of the methods, the sample may be contacted with a LOX-1 antibody or epitope binding fragment thereof and a CD66h ligand. Still other ligands that identify neutrophils generally may be useful in this context.

In one embodiment, therefore, the method involves contacting the biological sample with the ligand for CD15 prior to, or simultaneously with, the use of the LOX-1 antibody or epitope binding fragment thereof. In one embodiment, therefore, the method involves contacting the biological sample with a ligand for CD66h prior to, or simultaneously with, the use of the LOX-1 antibody or epitope binding fragment thereof. In one embodiment, therefore, the method involves contacting the biological sample with a ligand for CD14 prior to, or simultaneously with, the use of the LOX-1 antibody or epitope binding fragment thereof. In one embodiment, therefore, the method involves contacting the biological sample with a ligand for CD11b prior to, or simultaneously with, the use of the LOX-1 antibody or epitope binding fragment thereof. In one embodiment, therefore, the method involves contacting the biological sample with the ligand for CD33, prior to, or simultaneously with, the use of the LOX-1 antibody or epitope binding fragment thereof. In one embodiment, therefore, the method involves contacting the biological sample with a ligand for CD14 and a ligand for CD15 prior to, or simultaneously with, the use of the LOX-1 antibody or epitope binding fragment thereof. In another embodiment, therefore, the method involves contacting the biological sample with a ligand for CD14, and a ligand for CD11b prior to, or simultaneously with, the use of the LOX-1 antibody or epitope binding fragment thereof. In another embodiment, therefore, the method involves contacting the biological sample with a ligand for CD14 and a ligand for CD33 prior to, or simultaneously with, the use of the LOX-1 antibody or epitope binding fragment thereof. In another embodiment, therefore, the method involves contacting the biological sample a ligand for CD15 and a ligand for CD11b prior to, or simultaneously with, the use of the LOX-1 antibody or epitope binding fragment thereof. In another embodiment, therefore, the method involves contacting the biological sample with a ligand for CD15 and a ligand for CD33 prior to, or simultaneously with, the use of the LOX-1 antibody or epitope binding fragment thereof. In another embodiment, therefore, the method involves contacting the biological sample with a ligand for CD15, a ligand for CD11b and a ligand for CD33 prior to, or simultaneously with, the use of the LOX-1 antibody or epitope binding fragment thereof. In another embodiment, therefore, the method involves contacting the biological sample with a ligand for CD14, a ligand for CD11b and a ligand for CD33 prior to, or simultaneously with, the use of the LOX-1 antibody or epitope binding fragment thereof.

In one embodiment of the method, any of these biomarkers may be detected prior to, or simultaneously with, the detection of the LOX-1 biomarker. The use of these other ligands assists in identifying all PMNs from other cells in the sample. Subsequent exposure of this population of cells from the sample with the LOX-1 ligands enables further separation of the PMN-MDSCs from the PMN population.

In one embodiment, following contact with the LOX-1 antibody or epitope binding fragment thereof and a second neutrophil specific biomarker ligand, such as a CD15 ligand or CD66b ligand, one may calculate the number of LOX-1+ vs. CD15+ or the number of LOX-1+ vs. CD66b+ cells are present in the sample. Such calculation can involve cell counting systems known to those of skill in the art.

In another embodiment, the method involves collecting as a second population, the cells which did not form complexes with the ligands, e.g., are not providing a detectable signal or are not immobilized on the substrate. This second population contains PMNs and other cells substantially free from PMN-MDSCs.

In still another embodiment, the methods described herein permit the obtaining of a population of cells enriched in human polymorphonuclear myeloid derived suppressor cells (PMN-MDSCs) by isolating from a cell suspension those cells which express LOX-1 to provide a population of cells enriched with PMN-MDSCs.

In still another embodiment, the methods involve measuring the amount/concentration of LOX-1 (for example, soluble LOX-1) in the serum and correlating that amount/concentration with the number of LOX-1+ PMN-MDSC.

These methods also permit the removal of human PMN-MDSCs from a cell population, comprising isolating from the cell population those cells which express LOX-1.

These methods are useful in one embodiment for monitoring of the progression or metastasis of a cancer or the monitoring of therapy in a cancer patient by permitting the evaluation of an increase in the LOX-1 cell surface receptor in a biological fluid of a patient having a cancer or under treatment for cancer. The increase of LOX-1+ cell number is indicative of metastasizing cancer or a progression of cancer. In other embodiments, this method may be useful diagnostically to initially detect the presence of cancer.

These methods depend initially upon obtaining an accurate enumeration or concentration of a PMN-MCSC cell population, substantially free of any PMNs, from a suitable biological sample of a subject. In one embodiment, these methods of determining an accurate cell count/concentration of cells expressing LOX-1 in a subject having a cancer or being treated for a cancer can be used to monitor the progression of the cancer (with or without treatment).

In still another embodiment, the use of these methods to determine an accurate measurement of LOX-1+ cells enable the monitoring of metastasis in a cancer, e.g., an increase in the LOX-1+ cell number indicates metastatic cancer. In another embodiment, these methods are useful to monitor and/or influence cancer treatment. For example, where the LOX-1+ cell number is increasing prior to cancer therapy, and subsequent performance of the method on a similar sample in the subject does not show a decrease in LOX-1+ cell number, the method can indicate that a change in therapeutic method or dosage is necessary.

In still another aspect, the method of measuring the LOX-1+ population in a fluid sample, such as whole blood, can be employed as a research method to determine the cause of the increase in such cells during the progression of a cancer.

In still other aspects of the diagnostic methods identified above, additional diagnostic steps include contacting the sample with a reagent that identifies activators or regulators of ER stress response in said cells. In one embodiment, the activators or regulators so identified are one or more of sXBP1, DDIT3 (CHOP), ATF4, ATF3, SEC61A ARGI or NOS-2. In another embodiment, the regulators are one or more of one or more of MYCN, CSF3, IL3, TGFβ1, TNF, LDL, RAF1, APP, IL6 PDGFBB, EPO, CD40LG, NFkB, IL13, AGT, IL1β, ERBB2, MAP2K1, VEGFα, CSF1, FLI1, or IFNγ.

Yet another embodiment of a diagnostic method for a mammalian subject with a cancer comprises the additional step of determining the size of a tumor in the subject by correlation with the number of LOX-1+ PMN or PMN-MDSC detected. This method step includes obtaining a biological sample from the subject; detecting whether LOX-1 is present in the sample by contacting the sample with an antibody or epitope binding fragment thereof as described herein; and detecting and distinguishing the complexes of antibody-bound LOX-1-cells from other cells not bound to the antibody in the sample. The size of the tumor is then determined based upon the increase of LOX-1+ PMNs or PMN-MDSCs over a baseline level. The baseline level is readily determined based upon enumeration of patient samples to create a standard.

The presence of LOX-1 (and any of the PMN-MDSC signature biomarkers) in the sample may be detected using any assay format known in the art or described herein. There are a variety of assay formats known to the skilled artisan for using the antibody or epitope binding fragment thereof and optionally a ligand to detect a signature biomarker in a sample. For example, see Harlow and Lane, Antibodies: A Laboratory Manual, Cold Spring Harbor Laboratory, 1988. In general, the presence or absence of LOX-1 in a sample may be determined by (a) contacting the sample with the antibody or epitope binding fragment thereof; and (b) determining the presence or level of LOX-1 in the sample, wherein the presence of LOX-1 in the sample is indicative of cancer or where an increase in the level of LOX-1 in the sample as compared to a control, is indicative of cancer.

Methods of detection, diagnosis, monitoring, and prognosis of cancer, or the status of cancer, and for the identification of subjects with an increased risk of cancer metastasis by detecting the presence of, or measuring the level of, LOX-1 protein or another biomarker described herein. Such methods may employ the antibodies or epitope binding fragments thereof as described herein. The particular assay format used to measure the LOX-1 in a biological sample may be selected from among a wide range of immunoassays, such as enzyme-linked immunoassays, sandwich immunoassays, homogeneous assays, immunohistochemistry formats, an enzyme linked immunosorbent assay (ELISA), a lateral flow assay, a radioimmunoassay (RIA), Fluorescence-activating cell sorting (FACS), a western blot, an immunoprecipitation, or other conventional assay formats. One of skill in the art may readily select from any number of conventional immunoassay formats to perform this invention. Other reagents for the detection of protein in biological samples, such as peptide mimetics, synthetic chemical compounds capable of detecting LOX-1 may be used in other assay formats for the quantitative detection of LOX-1 protein in biological samples, such as high-pressure liquid chromatography (HPLC), immunohistochemistry, etc.

The diagnostic methods described herein can employ contacting a patient's sample with a diagnostic reagent, as described above, which forms a complex or association with LOX-1 in the patients' sample. Detection or measurement of the sample LOX-1 may be obtained by use of a variety of apparatus or machines, such as computer-programmed instruments that can transform the detectable signals generated from the diagnostic reagents complexed with the LOX-1 or other biomarker in the biological sample into numerical or graphical data useful in performing the diagnosis. Such instruments may be suitably programmed to permit the comparison of the measured LOX-1 in the sample with the appropriate reference standard and generate a diagnostic report or graph.

In another aspect, provided is a method of evaluating differentiation of polymorphonuclear myeloid derived suppressor cells (PMN-MDSCs) from polymorphonuclear neutrophils (PMNs) in a biological sample containing both types of cells comprising: contacting the sample with C-reactive protein (CRP), or Oxidized-low density lipoprotein (ox-LDL), or other agent which induces differentiation of PMN-MDSC, or activators or regulators of ER stress response; and detecting and optionally collecting PMN-MDSCs using one or more of the antibody or epitope binding fragment thereof as described herein. In certain embodiments, the agent is identified as an antagonist or inhibitor of PMN-MDSC differentiation if treatment with the agent decreases expression of LOX-1 or numbers/concentration of LOX-1 positive cells/neutrophils/PMNs compared to a no-treatment control. In certain embodiments, the agent is identified as an activator of PMN-MDSC differentiation if treatment with the agent increases expression of LOX-1 or numbers/concentration of LOX-1 positive cells/neutrophils/PMNs compared to a no-treatment control.

Also provided is an assay method of identifying an antagonist or inhibitor of LOX-1 expression, comprising contacting a biological sample or a cell population of PMN-MDSCs and PMNs with a test agent; and detecting expression of LOX-1 using one or more of the antibody or epitope binding fragment thereof as described herein. In certain embodiments, the test agent is identified as an antagonist or inhibitor of LOX-1 expression if treatment with the test, agent decreases expression of LOX-1 or LOX-1 positive cells/neutrophils/PMNs compared to a no-treatment control.

### B. Therapeutic Methods

In one aspect, a therapeutic method involves administering to a patient in need thereof an effective amount of a composition that reduces or inhibits ER stress response in mammalian neutrophils or reduces or inhibits LOX-1 expression on neutrophil populations In another embodiment, the antibodies and fragments described herein are useful in an amount that reduces or inhibits the growth or metastasis of cancer cells that express LOX-1. In another embodiment, the amounts of the compositions containing the antibodies, fragments, or inhibits is effective to reduce the growth, spread or amount of LOX-1+ cells in any disorder characterized by cells expressing LOX-1. In one embodiment, the patient has a LOX-1+ associated cancer. In another embodiment, the patient has a LOX-1+ associated disease.

In another aspect, a method of treating a cancer in a subject is provided comprising administering an effective amount of one or more of the antibody or epitope binding fragments thereof as described herein, with an additional inhibitor of LOX-1 expression or inhibitor of the ER stress response. In a further embodiment, B-I09 is administered to the subject. In other embodiments, the composition that reduces or inhibits the ER stress response comprises one or more of the antibody or epitope binding fragment thereof as described herein, and/or an antibody or functional antigen-binding fragment that binds to or inhibits the expression, activity or activation of at least one of sXBP1, DDIT3 (CHOP), ATF4, ATF3, SEC61A ARGI, MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, Fin, CD15, CD66b or CD33.

In still other embodiments, the treatment step may be combined with the diagnostic steps in a combined method. Such a method combines diagnosing and treating a cancer and combines the steps, such as obtaining a biological sample from a subject; detecting whether PMN-MDSC are present in the sample via detecting LOX-1 expressing cells/neutrophils/PMNs; diagnosing the subject with cancer when the presence of LOX-1+ (optionally with any other of the PMN-MDSC signature biomarkers identified herein or in the references cited) is detected at a level that indicates PMN-MDSC are present; and administering an effective amount of a composition that reduces or inhibits ER stress response in mammalian neutrophils or reduces or inhibits LOX-1 expression on neutrophil populations.

In another aspect, provided is a method of treating a cancer in a subject comprising: obtaining a biological sample from a subject; contacting the sample with one or more of the antibody or epitope binding fragment thereof or the composition as described herein; detecting whether LOX-1 positive cells are present in the sample; and the presence of LOX-1+ cell is detected, administering an effective amount of a composition that reduces or inhibits ER stress response in mammalian neutrophils or reduces or inhibits LOX-1 expression on neutrophil populations (for example, B-I09), and/or an identified LOX-1 inhibitor, and/or an identified PMN-MDSC differentiation inhibitor, and/or the antibody or epitope binding fragment thereof. In certain embodiments, the LOX-1 positive cells are LOX-1 positive neutrophils. In certain embodiments, the LOX-1 positive cells are LOX-1 positive MDSC. In certain embodiments, the LOX-1 positive cells are PMN-MDSC.

In yet another aspect, provided is a method of treating a cancer in a subject comprising: obtaining a biological sample from a subject; contacting the sample with one or more of the antibody or epitope binding fragment thereof or the composition as described herein; detecting whether PMN-MDSC are present in the sample; and the presence of LOX-1+ is detected at a level that indicates PMN-MDSC are present, administering an effective amount of a composition that reduces or inhibits ER stress response in mammalian neutrophils or reduces or inhibits LOX-1 expression on neutrophil populations, and/or an identified LOX-1 inhibitor, and/or an identified PMN-MDSC differentiation inhibitor, and/or the antibody or epitope binding fragment thereof hi certain embodiments, the composition that reduces or inhibits the ER stress response comprises one or more of the antibody or epitope binding fragment thereof as described herein, and/or an antibody or functional antigen-binding fragment that binds to or inhibits the expression, activity or activation of at least one of sXBP1, DDIT3 (CHOP), ATF4, ATF3, SEC61A ARGI, MYCN, CSF3, IL3, TGFβ1, TNF, LDL, RAF1, APP, IL6 PDGFBB, EPO, CD40LG, Nek, IL13, AGT, IL1β, ERBB2, MAP2K1, VEGFα, CSF1, FLI1, Fin, CD15, CD66b or CD33.

In certain embodiments, provided is a method of treating a cancer in a subject comprising obtaining a biological sample from the subject; removing and deleting LOX-1+ PMN-MDSC with an effective amount of one or more of the antibody or epitope binding fragment thereof or a composition as described herein; and administering the biological sample substantially free from PMN-MDSCs to the subject.

Whether the LOX-1+ antibodies and/or epitope binding fragments are administered alone to treat the LOX-1+ associated cancer or disease, or are coupled with diagnostic and therapeutic steps as described above, the LOX-1+ antibodies and/or fragments may be administered by any suitable method or route. Similarly, the same or other routes can be used to co-administer other active drugs or therapies in conjunction with the composition described herein. Routes of administration include, for example, systemic, oral, intravenous, intraperitoneal, subcutaneous, intramuscular, or intratumor administration. In certain embodiments, administration involves directly introducing the compositions as described into a tumor microenvironment. In still other embodiments the LOX-1 antibodies or fragments may be coupled with other therapeutic moieties to target the other therapeutic moieties to the environment of LOX-1+ expressing cells or tissues.

### Examples

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill iii the art will understand that certain changes or variations can be made in the disclosed embodiments of the examples and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1: LOX1 Mab Development

Monoclonal antibodies against LOX-1 is developed as summarized as follows and further detailed in the Examples: (1) Lox-1 Immunogen was generated including construction of DNA and production of recombinant Lox1 protein; (2) animal was immunized and seroconversion was evaluated; (3) spleen cells were isolated and antibody secreting cells were enriched; (4) antibody secreting cells were fused with myeloma cells followed by hybridoma positive selection and expansion screen for specificity; (5) variable regions of both heavy and light chains were amplified via PCR and sequenced; and (6) characterization and modification of heavy and light chains were performed for large scale amplification.

Construction of Human Lox1 DNA

The human LOX1 plasmid DNA construct was synthesized. The DNA immunogen was computationally aligned and optimized via codon and RNA optimization for enhanced expression. Additionally, an IgE leader sequence (aa 1-18 of SEQ ID NO: 1) was inserted to enhance immunogen expression. This optimized hLOX1 immunogen (with amino acid sequence of SEQ ID NO: 1) was constructed by subcloning into pMV101 expression vector with the cytomegalovirus immediate-early promoter. The large-scale DNA production was carried out and DNA gel electrophoresis technique used to separate DNA fragments to confirm the insert.

Generation and Characterization of hLOX1 Recombinant Protein.

A recombinant protein spanning the entire human LOX1 sequence was synthesized in-house since commercial reagents only encode portions of the full-length protein. The hLOX1 immunogen was cloned into pET30a Escherichia coli expression vector and produced. Nickel column chromatography method was used to purify the recombinant hLOX1 protein. SDS-PAGE was used to analyze the predicted size of overexpressed proteins in lysates using anti-His tag antibodies.

DNA Immunization and Electroporation Delivery in Mice

Six- to eight-week old female BALB/c mice (The Jackson Laboratory, ME, USA) were housed in the Wistar Institute Animal Facility in a light-cycled, temperature- and humidity-controlled condition. All animal studies were performed in accordance with the recommendations and guidelines from the National Institute of Health (NIH) and the Wistar Institute Institutional Animal Care and Use Committee.

Mice were injected by a 30 cc syringe with 50 ug of DNA diluted in 30 ug of sterile water into the anterior tibialis (TA) muscle. The injection site is immediately electroporated using the CELLECTRA electroporation (EP) delivery device (Inovio Pharmaceuticals, PA, USA). This 26-gauge, stainless steel three-pronged probe is inserted 2 mm into the injection site into the TA muscle and delivered 0.1 Amps of triangulated square-wave pulses for 52 msec twice. Blood via the submandibular method is collected prior to the DNA injection and EP delivery to assess antibody levels in circulation. Mice under all experimental manipulations were anesthetized with 2-5% isoflurane (Phoenix, Mo., USA).

In addition to the DNA immunization as described above, mice were subcutaneously injected 5 ug of hLOX1 recombinant protein mixed with incomplete Freund's adjuvant (Sigma, USA) in a total volume of 100 ul per injection.

All animal studies were performed in accordance with the recommendations and guidelines from the National Institute of Health (NIH) and the Wistar Institute Institutional Animal Care and Use Committee.

ELISA for the Detection of Antibodies and Seroconversion

Flat-bottomed MaxiSorp 96-well ELIS plates (ThermoFisher, USA) were coated with 1 μg/ml of hLOX1 recombinant protein diluted in PBS and incubated at 4° C. overnight. Plates were washed 4 times with PBS buffer solution (PBS+ 0.01% Tween-20 (ThermoFisher, USA)) and blocked with 10% FBS diluted in PBS for 1 hour at room temperature. On a separate, non-absorbent U-bottom 96-well plate, serum samples are diluted on a half-log scale starting with the initial dilution of 1:50. One hundred microliter (μl) of the diluted samples in each well are transferred to the Maxisorp plate after 5 washes. The plate is incubated for 2-hours room temperature, then washed 5 times with PBST. The plate is then incubated with HRP-labeled goat anti-mouse IgG (Sigma-Aldrich, USA) for one hour. The plate is washed 5 times and 100 μl of 3,3′5,5′-Tetramethylbenzidine (TMB) Substrate (Sigma-Aldrich, USA) is added to each well for 10 minutes before the reaction being stopped with 2M H₂SO₄ solution. The plate is read at 450 nm by Biotek ELISA plate reader. The antibody endpoint titer is defined as the highest dilution of a sample with OD values >(mean+3SD) of vehicle sample. Samples with a titer less than 50 were given the endpoint titer of 1.

Lox-1 Hybridoma Fusion & Characterization of Specificity of hLOX-1 mAbs

Based on the ELISA result for the highest-binding sera, immunogen-boosted mice were preliminarily screened for the highest OD value as well as the highest endpoint titer then sacrificed to harvest the spleen. Single-cell suspensions of splenocytes in RIO were prepared using a Stomacher 80 paddle blender (AJ. Seward and Co. Ltd.) for 30 seconds on high speed. The samples were filtered through 45 mm nylon filters and the cell pellets were collected at 1,500 g centrifuged 10 min at 4° C. The collected cell pellets were lysed using ammonium-chloride-potassium lysis buffer (ACK) (Life Technologies, USA). The splenocytes in single-cell suspension was sent to Fox Chase Cell Culture Center (Philadelphia, Pa.) for a fusion process with P3X63.Ag.6.5.3 myeloma cells. The initial fusion produces approximately 1400 hybridoma candidates. After antibody screening through ELISA as previously mentioned, approximately 50-60 antibody-producing mouse hybridoma clones were identified as having antibody binding at least four-fold greater than the background level reactivity. The cell lines of top 5 hybridoma candidates are amplified, and the extracted RNA of each cell line is sequenced for HL and V_{L} variable regions using PCR.

Total RNA was isolated from the hybridoma cells following the technical manual of TRIzol® Reagent (TRIzol® Reagent (Ambion, Cat. No. 15596-026). Total RNA was then reverse-transcribed into cDNA using either isotype-specific anti-sense primers or universal primers with 1st Strand cDNA Synthesis Kit. (PrimeScript™ 1st Strand cDNA Synthesis Kit, Takara, Cat. No.: 6110A). Antibody fragments of heavy chain and light chain were amplified by rapid amplification of cDNA ends (RACE). Amplified antibody fragments were cloned into a standard cloning vector separately. Colony PCR was performed to screen for clones with inserts of correct sizes. Individual positive clones with correct VH and VL insert sizes were sequenced.

Comparison of the hLox-1 IgG Avidity Test Results for Hybridoma.

Microtiter plates previously coated with recombinant Lox-1 antigens were washed 3 times with PBS plus 0.05% tween 20 (PBST). Hybridomas 1B6, 2A10, 3D8, 3E6, 4D6, 5C2, 5F5, 6A10, 8E7, 9E12, 10H11, 11A6, 12D9, 11C5, 12A10, 12E4, 14A12, and 12G6 samples were diluted as indicated and added (100 μl/well). The avidity of antibodies against hLOX-1 protein was determined by a particle disruption ELISA using 4M Urea. The binding of hLOX1 protein and incubation with mAbs were performed followed by a 5-minute incubation with urea after washing five times with 0.05% PBST. Control (urea-untreated) wells were treated with PBS during the 5-minute incubation. The anti-human IgG conjugated with horseradish peroxidase (HRP) was added with the dilution of 1/5000 in PBS for 1 hour. Afterwards, the plate is washed 5 times, and 100 μl of 3,3′5,5′-Tetramethylbenzidine (TMB) Substrate (Sigma-Aldrich, USA) is added to each well for 10 minutes before the reaction being stopped with 2M H₂SO₄ solution. The plate is read at 450 nm by Biotek ELISA plate reader.

Binding Properties of hLOX-1 mAbs-Western Blot Analysis

Immunological reactivity of hLox-1 antibodies was characterized by Western blot analysis against native and denatured full-length recombinant proteins. Protein samples were electrophoretically separated under native or denaturing conditions on SDS-PAGE, performed using Novex gels with reagents as followed: 4-10% mini-gel, 10× Native-PAGE running buffer and 2× Native-PAGE: sample buffer. Denatured protein was heated in a heat block for 10 minutes at 57° C. Two micrograms (2 μg) of recombinant full length hLOX1 protein or non-specific POWV-Env viral protein was loaded to each well along with a protein size marker as a ladder. Gels were run at 150 V for 1 hour in a running buffer. Protein was transferred to a nitrocellulose membrane using the iBlot 2 Gel Transfer Device (Life Technologies). Membranes were blocked in Odyssey blocking buffer (Licor) for 1 hour at room temperature. Indicated hybridomas were diluted 1:250 in 0.5× Odyssey blocking buffer with 0.1% Tween-20 (BioRad) and incubated with the membranes overnight at 4° C. Membranes were washed and then incubated with the appropriate secondary antibody (goat anti-mouse IRDye680RD) for hybridoma samples for 1 hour at room temperature. After washing with PBST 5 times, membranes were imaged on the Odyssey infrared imager.

In summary, an immunogen strategy with a DNA immunization followed by a recombinant protein boost approach was developed to generate monoclonal antibodies (mAbs) against human LOX-1. Over 1000 clones were screened and 20 monoclones of binders were identified by ELISA. The antibodies were then characterized for Avidity and conformational binding by ELISA and Western Blotting (WB). Clones 6A10 and 12D9 were finalized as having strong avidity. Also, the preserved binding epitopes are highly conserved by both native as well as denatured conditions.

All the group of clones are studied in Fluorescence-activated cell sorting (FACS) against human cell targets. Without wishing to be bound by the theory, native conditions are likely most important for Fluorescence-activated cell sorting (FACS), cell staining and functional assays, while WB denatured antibodies likely more important in diagnostic assays.

Functionality of antibodies and their ability to function in immune assays are investigated in cancer models described below.

### Example 2: Uses of Anti-LOX1 Antibodies

Expression of LOX1 on PMN-MDSC by flow cytometry using the antibodies described herein or another available anti-LOX-1 monoclonal antibody (for example, clone 15C4; Biolegend Inc., San Diego, Calif.) is analyzed in blood samples from patients different types of cancer, for example, head and neck, breast, non-small lung, or colon cancer. Blood samples form healthy donors are served as negative control. PMN-MSC are identified using classical definition, i.e., cells with CD11b+ CD14− CD15+ and CD33+ from the low-density mononuclear cells fraction.

Additionally, blood sample from heathy donor and cancer patients are incubated with C-reactive protein (CRP), or Oxidized-low density lipoprotein (ox-LDL) or other factors which induces expression of LOX1 (for example, as identified in US Patent Application Publication with Publication No. 20180059115). The antibodies as described herein are then used to evaluate up-regulation of LOX1 expression.

Further, the antibodies as described herein are used to treat LOX1+ PMN together with a factor which induces expression of LOX1. LOX1 expression and ROS production are then evaluated.

In certain embodiments, neutrophils from healthy donors are treated with 100 ug/ml immune complexes (for example, one or more of the described LOX-1 antibodies) for 18 hours to induce upregulation of LOX-1.

In certain embodiments, one or more of the LOX-1 antibodies or epitope binding fragments is added at different concentrations (from 5 μg to 50 μg/ml) together with a reagent to measure reactive oxygen species (ROS) response (for example, 2′,7′—dichlorofluorescin diacetate, i.e., DCFDA). 30 minutes later, LOX-1 ligand oxidized LDL is added, and ROS production is measured. Reduction in ROS production indicates anti-ROS effects of the antibody or epitope binding fragment.

Suppressive activity of PMN-MDSC is assessed in mixed leukocyte reaction where T cells from one donor are incubated at 10:1 ratio with dendritic cells from another donor. PMN-MDSC after treatment described above are added at different ratios to T cells (1:1-1:4) and T cell proliferation is measured 5 days later using ³H-thymidine uptake.

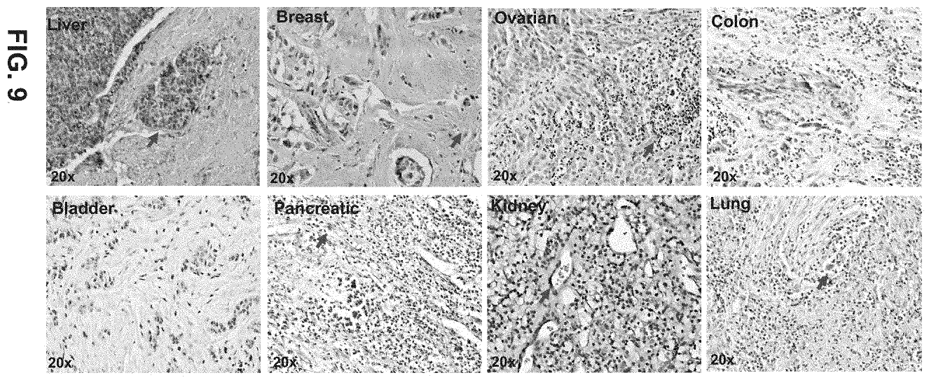

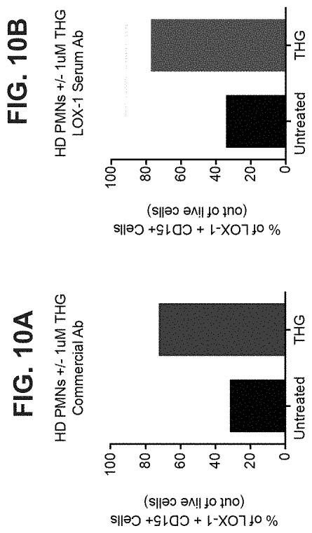

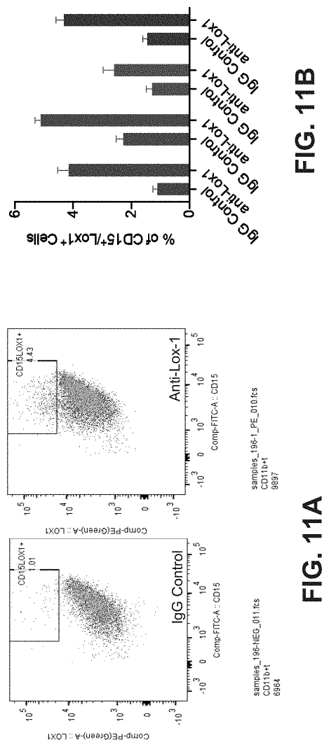

An immunohistochemical analysis of LOX- expression in tumor tissues was performed. 5 μm sized paraffin embedded tissue sections were deparaffinized. Standard IHC protocol was followed to stain the tumor tissue samples using the monoclonal antibody against LOX-1 (IgG1) for the detection of LOX-1 followed by staining with hematoxylin-eosin. LOX-1 expression was observed in tumor tissues from the liver, breast, ovarian, pancreatic, kidney and lung tissues at 20×, but not in colon or bladder tumor tissues. (see FIG. 9).

All documents, including websites, cited in this specification are incorporated herein by reference. The Sequence Listing filed herewith, labelled “WST183PCT_ST25.txt”, and the sequences and text therein are incorporated by reference. While invention has been described with reference to particular embodiments, it will be appreciated that modifications can be made without departing from the spirit of the invention. Such modifications are intended to fall within the scope of the appended claims.

TABLE 6
(Sequence Listing Free Text)
The following information is provided for sequences
containing free text under numeric identifier <223>.

| SEQ ID NO: | |
|---|---|
| (containing | |
| free text) | Free text under <223> |
| 1 | <223> recombinant hLOX-1 protein |
| | <222> (1)..(18) |
| | <223> Signal peptide |
| | <222> (19)..(290) |
| | <223> human LOX-1 protein |
| 2 | <223> Coding sequence for variable region of the 6A10 |
| | heavy chain. |
| | <222> (1)..(57) |
| | <223> signal sequence |
| | <222> (58)..(147) |
| | <223> Framework 1 |
| | <222> (148)..(162) |
| | <223> CDR1 |
| | <222> (163)..(204) |
| | <223> Framework 2 |
| | <222> (205)..(255) |
| | <223> CDR2 |
| | <222> (256)..(351) |
| | <223> Framework 3 |
| | <222> (352)..(363) |
| | <223> CDR3 |
| | <222> (364)..(396) |
| | <223> Framework 4 |
| 3 | <223> Amino acid sequence of variable region of the 6A10 |
| | heavy chain. |
| | <222> (1)..(19) |
| | <223> signal seqeunce |
| | <222> (20)..(49) |
| | <223> FR1 |
| | <222> (50)..(54) |
| | <223> CDR1 |
| | <222> (55)..(68) |
| | <223> FR2 |
| | <222> (69)..(85) |
| | <223> CDR2 |
| | <222> (86)..(117) |
| | <223> FR3 |
| | <222> (118)..(121) |
| | <223> CDR3 |
| | <222> (122)..(132) |
| | <223> FR4 |
| 4 | <223> Coding sequence for variable region of the 6A10 |
| | light chain. |
| | <222> (1)..(60) |
| | <223> signal sequence |
| | <222> (61)..(129) |
| | <223> Framework 1 |
| | <222> (130)..(162) |
| | <223> CDR1 |
| | <222> (163)..(207) |
| | <223> Framework 2 |
| | <222> (208)..(228) |
| | <223> CDR2 |
| | <222> (229)..(324) |
| | <223> Framework 3 |
| | <222> (325)..(354) |
| | <223> CDR3 |
| | <222> (355)..(384) |
| | <223> Framework 4 |
| 5 | <223> Amino acid sequence of variable region of the 6A10 |
| | light chain. |
| | <222> (1)..(20) |
| | <223> signal seqeunce |
| | <222> (21)..(43) |
| | <223> FR1 |
| | <222> (44)..(54) |
| | <223> CDR1 |
| | <222> (55)..(69) |
| | <223> FR2 |
| | <222> (70)..(76) |
| | <223> CDR2 |
| | <222> (77)..(108) |
| | <223> FR3 |
| | <222> (109)..(118) |
| | <223> CDR3 |
| | <222> (119)..(128) |
| | <223> FR4 |
| 6 | <223> Coding sequence for variable region of the 9E12 |
| | heavy chain. |
| | <222> (1)..(57) |
| | <223> signal sequence |
| | <222> (58)..(147) |
| | <223> FR1 |
| | <222> (58)..(147) |
| | <223> FR1 |
| | <222> (148)..(162) |
| | <223> CDR1 |
| | <222> (163)..(204) |
| | <223> FR2 |
| | <222> (205)..(255) |
| | <223> CDR2 |
| | <222> (256)..(351) |
| | <223> FR3 |
| | <222> (352)..(360) |
| | <223> CDR3 |
| | <222> (361)..(393) |
| | <223> FR4 |
| 7 | <223> Amino acid sequence of variable region of the 9E12 |
| | heavy chain. |
| | <222> (1)..(19) |
| | <223> Signal |
| | <222> (20)..(49) |
| | <223> FR1 |
| | <222> (50)..(54) |
| | <223> CDR1 |
| | <222> (55)..(68) |
| | <223> FR2 |
| | <222> (69)..(85) |
| | <223> CDR2 |
| | <222> (86)..(117) |
| | <223> FR3 |
| | <222> (118)..(120) |
| | <223> CDR3 |
| | <222> (121)..(131) |
| | <223> FR4 |
| 8 | <223> Coding sequence for variable region of the 9E12 |
| | light chain. |
| | <222> (1)..(57) |
| | <223> Signal |
| | <222> (58)..(126) |
| | <223> FR1 |
| | <222> (127)..(174) |
| | <223> CDR1 |
| | <222> (175)..(219) |
| | <223> FR2 |
| | <222> (220)..(240) |
| | <223> CDR2 |
| | <222> (241)..(336) |
| | <223> FR3 |
| | <222> (337)..(363) |
| | <223> CDR3 |
| | <222> (364)..(393) |
| | <223> FR4 |
| 9 | <223> Amino acid sequence of variable region of the 9E12 |
| | light chain |
| | .<222> (1)..(19) |
| | <223> signal peptide |
| | <222> (20)..(42) |
| | <223> FR1 |
| | <222> (43)..(58) |
| | <223> CDR1 |
| | <222> (59)..(73) |
| | <223> FR2 |
| | <222> (74)..(80) |
| | <223> CDR2 |
| | <222> (81)..(112) |
| | <223> FR3 |
| | <222> (113)..(121) |
| | <223> CDR3 |
| | <222> (122)..(131) |
| | <223> FR4 |
| 10 | <223> Coding sequence for variable region of the 12D9 |
| | heavy chain. |
| | <222> (1)..(57) |
| | <223> signal |
| | <222> (58)..(147) |
| | <223> FR1 |
| | <222> (148)..(162) |
| | <223> CDR1 |
| | <222> (163)..(204) |
| | <223> FR2 |
| | <222> (205)..(255) |
| | <223> CDR3 |
| | <222> (256)..(351) |
| | <223> FR3 |
| | <222> (352)..(378) |
| | <223> CDR3 |
| | <222> (379)..(411) |
| | <223> FR4 |
| 11 | <223> Amino acid sequence of variable region of the 12D9 |
| | heavy chain. |
| | <222> (1)..(19) |
| | <223> signal |
| | <222> (20)..(49) |
| | <223> FR1 |
| | <222> (50)..(54) |
| | <223> CDR1 |
| | <222> (55)..(68) |
| | <223> FR2 |
| | <222> (86)..(117) |
| | <223> FR3 |
| | <222> (118)..(126) |
| | <223> CDR3 |
| | <222> (127)..(137) |
| | <223> FR4 |
| 12 | <223> Coding sequence for variable region of the 12D9 |
| | light chain. |
| | <222> (1)..(60) |
| | <223> signal |
| | <222> (61)..(129) |
| | <223> FR1 |
| | <222> (130)..(162) |
| | <223> CDR1 |
| | <222> (163)..(207) |
| | <223> FR2 |
| | <222> (208)..(228) |
| | <223> CDR2 |
| | <222> (229)..(324) |
| | <223> FR3 |
| | <222> (325)..(351) |
| | <223> CDR3 |
| | <222> (352)..(381) |
| | <223> FR4 |
| 13 | <223> Amino acid sequence of variable region of the 12D9 |
| | light chain |
| | <222> (1)..(20) |
| | <223> signal |
| | <222> (21)..(43) |
| | <223> FR1 |
| | <222> (44)..(54) |
| | <223> CDR1 |
| | <222> (55)..(69) |
| | <223> FR2 |
| | <222> (70)..(76) |
| | <223> CDR2 |
| | <222> (77)..(108) |
| | <223> FR3 |
| | <222> (109)..(117) |
| | <223> CDR3 |
| | <222> (118)..(127) |
| | <223> FR4 |
| 14 | <223> Coding sequence for variable region of the 12E4 |
| | heavy chain. |
| | <222> (1)..(57) |
| | <223> Signal |
| | <222> (58)..(147) |
| | <223> FR1 |
| | <222> (148)..(162) |
| | <223> CDR1 |
| | <222> (163)..(204) |
| | <223> FR2 |
| | <222> (205)..(255) |
| | <223> CDR2 |
| | <222> (256)..(351) |
| | <223> FR3 |
| | <222> (352)..(375) |
| | <223> CDR3 |
| | <222> (376)..(408) |
| | <223> FR4 |
| 15 | <223> Amino acid sequence of variable region of the 12E4 |
| | heavy chain. |
| | <222> (1)..(19) |
| | <223> signal |
| | <222> (20)..(49) |
| | <223> FR1 |
| | <222> (50)..(54) |
| | <223> CDR1 |
| | <222> (55)..(68) |
| | <223> FR2 |
| | <222> (69)..(85) |
| | <223> CDR2 |
| | <222> (86)..(117) |
| | <223> FR3 |
| | <222> (118)..(125) |
| | <223> CDR3 |
| | <222> (126)..(136) |
| | <223> FR4 |
| 16 | <223> Coding sequence for variable region of the 12E4 |
| | light chain. |
| | <222> (1)..(57) |
| | <223> signal |
| | <222> (58)..(126) |
| | <223> FR1 |
| | <222> (127)..(159) |
| | <223> CDR1 |
| | <222> (160)..(204) |
| | <223> FR2 |
| | <222> (205)..(225) |
| | <223> CDR2 |
| | <222> (226)..(321) |
| | <223> FR3 |
| | <222> (322)..(348) |
| | <223> CDR3 |
| | <222> (349)..(378) |
| | <223> FR4 |
| 17 | <223> Amino acid sequence of variable region of the 12E4 |
| | light chain. |
| | <222> (1)..(19) |
| | <223> signal |
| | <222> (20)..(42) |
| | <223> FR1 |
| | <222> (43)..(53) |
| | <223> CDR1 |
| | <222> (54)..(68) |
| | <223> FR2 |
| | <222> (69)..(75) |
| | <223> CDR2 |
| | <222> (76)..(107) |
| | <223> FR3 |
| | <222> (108)..(116) |
| | <223> CDR3 |
| | <222> (117)..(126) |
| | <223> FR4 |
| 18 | <223> Coding sequence for variable region of the 4D6 |
| | heavy chain. |
| | <222> (1)..(57) |
| | <223> signal |
| | <222> (58)..(147) |
| | <223> FR1 |
| | <222> (148)..(162) |
| | <223> CDR1 |
| | <222> (168)..(204) |
| | <223> FR2 |
| | <222> (205)..(255) |
| | <223> CDR2 |
| | <222> (256)..(351) |
| | <223> FR3 |
| | <222> (352)..(375) |
| | <223> CDR3 |
| | <222> (376)..(408) |
| | <223> FR4 |
| 19 | <223> Amino acid sequence of variable region of the 4D6 |
| | heavy chain |
| | <222> (1)..(19) |
| | <223> signal |
| | <222> (20)..(49) |
| | <223> FR1 |
| | <222> (50)..(54) |
| | <223> CDR1 |
| | <222> (55)..(68) |
| | <223> FR2 |
| | <222> (69)..(85) |
| | <223> CDR2 |
| | <222> (86)..(117) |
| | <223> FR3 |
| | <222> (118)..(125) |
| | <223> CDR3 |
| | <222> (126)..(136) |
| | <223> FR4 |
| 20 | <223> Coding sequence for variable region of the 4D6 |
| | light chain, clone 1. |
| | <222> (1)..(60) |
| | <223> Signal |
| | <222> (61)..(129) |
| | <223> FR1 |
| | <222> (130)..(162) |
| | <223> CDR1 |
| | <222> (163)..(207) |
| | <223> FR2 |
| | <222> (208)..(228) |
| | <223> CDR2 |
| | <222> (229)..(324) |
| | <223> FR3 |
| | <222> (325)..(351) |
| | <223> CDR3 |
| | <222> (352)..(381) |
| | <223> FR4 |
| 21 | <223> Amino acid sequence of variable region of the 4D6 |
| | light chain, clone 1. |
| | <222> (1)..(20) |
| | <223> Signal |
| | <222> (21)..(43) |
| | <223> FR1 |
| | <222> (44)..(54) |
| | <223> CDR1 |
| | <222> (55)..(69) |
| | <223> FR2 |
| | <222> (70)..(76) |
| | <223> CDR2 |
| | <222> (77)..(108) |
| | <223> FR3 |
| | <222> (109)..(117) |
| | <223> CDR3 |
| | <222> (118)..(127) |
| | <223> FR4 |
| 22 | <223> Coding sequence for variable region of the 4D6 |
| | light chain, clone 2. |
| | <222> (1)..(60) |
| | <223> Signal |
| | <222> (61)..(129) |
| | <223> FR1 |
| | <222> (130)..(174) |
| | <223> CDR1 |
| | <222> (175)..(219) |
| | <223> FR2 |
| | <222> (220)..(240) |
| | <223> CDR2 |
| | <222> (241)..(336) |
| | <223> FR3 |
| | <222> (337)..(363) |
| | <223> CDR3 |
| | <222> (364)..(393) |
| | <223> FR4 |
| 23 | <223> Amino acid sequence of variable region of the 4D6 |
| | light chain, clone 2. |
| | <222> (1)..(20) |
| | <223> Signal |
| | <222> (21)..(43) |
| | <223> FR1 |
| | <222> (44)..(58) |
| | <223> CDR1 |
| | <222> (59)..(73) |
| | <223> FR2 |
| | <222> (74)..(80) |
| | <223> CDR2 |
| | <222> (81)..(112) |
| | <223> FR3 |
| | <222> (113)..(121) |
| | <223> CDR3 |
| | <222> (122)..(131) |
| | <223> FR4 |
| 24 | <223> Coding sequence for variable region of the 3D8 |
| | heavy chain. |
| | <222> (1)..(57) |
| | <223> signal |
| | <222> (58)..(147) |
| | <223> FR1 |
| | <222> (148)..(162) |
| | <223> CDR1 |
| | <222> (163)..(204) |
| | <223> FR2 |
| | <222> (205)..(255) |
| | <223> CDR2 |
| | <222> (256)..(351) |
| | <223> FR3 |
| | <222> (352)..(378) |
| | <223> CDR3 |
| | <222> (379)..(411) |
| | <223> FR4 |
| 25 | <223> Amino acid sequence of variable region of the 3D8 |
| | heavy chain. |
| | <222> (1)..(19) |
| | <223> Signal |
| | <222> (20)..(49) |
| | <223> FR1 |
| | <222> (50)..(54) |
| | <223> C DR1 |
| | <222> (55)..(68) |
| | <223> FR2 |
| | <222> (69)..(85) |
| | <223> CDR2 |
| | <222> (86)..(117) |
| | <223> FR3 |
| | <222> (118)..(126) |
| | <223> CDR3 |
| | <222> (127)..(137) |
| | <223> FR4 |
| 26 | <223> Coding sequence for variable region of the 3D8 |
| | light chain. |
| | <222> (1)..(60) |
| | <223> signal |
| | <222> (61)..(129) |
| | <223> FR1 |
| | <222> (130)..(162) |
| | <223> CDR1 |
| | <222> (163)..(207) |
| | <223> FR2 |
| | <222> (208)..(228) |
| | <223> CDR2 |
| | <222> (229)..(324) |
| | <223> FR3 |
| | <222> (325)..(351) |
| | <223> CDR3 |
| | <222> (352)..(381) |
| | <223> FR4 |
| 27 | <223> Amino acid sequence of variable region of the 3D8 |
| | light chain. |
| | <222> (1)..(20) |
| | <223> signal |
| | <222> (21)..(43) |
| | <223> FR1 |
| | <222> (44)..(54) |
| | <223> CDR1 |
| | <222> (55)..(69) |
| | <223> FR2 |
| | <222> (70)..(76) |
| | <223> CDR2 |
| | <222> (77)..(108) |
| | <223> FR3 |
| | <222> (109)..(117) |
| | <223> CDR3 |
| | <222> (118)..(127) |
| | <223> FR4 |
| 28 | <223> a coding sequence for hLOX-1 |

1. A recombinant antibody or an epitope binding fragment thereof that specifically binds to a lectin-like oxidized low-density lipoprotein (LDL) receptor-1 (LOX-1) epitope, said antibody or fragment comprising at least one of:
(a) one or more of heavy chain complementarity-determining regions (CDRs) encoded by CDR-coding sequences of 6A10, 3D8, 9E12, 12D9, 12E4 or 4D6 identified in Table 1, or a nucleic acid sequence at least 85% identical thereto; or
(b) one or more of heavy chain CDRs having CDR amino acid sequences of 6A10, 3D8, 9E12, 12D9, 12E4 or 4D6 identified in Table 2, or an amino acid sequence at least 85% identical thereto; or
(c) one or more of light chain CDRs encoded by CDR-coding nucleic acid sequences of 6A10, 3D8, 9E12, 12D9, 12E4 or 4D6 identified in Table 3, or a nucleic acid sequence at least 85% identical thereto; or
(d) one or more of light chain CDRs having CDR amino acid sequences of 6A10, 3D8, 9E12, 12D9, 12E4 or 4D6 clone 1 or 4D6 clone 2, identified in Table 4, or an amino acid sequence at least 85% identical thereto.

2. The recombinant antibody or an epitope binding fragment according to claim 1, said antibody or fragment comprising at least one of:
(a) a heavy chain variable region having an amino acid sequence of SEQ ID NOs: 3, 7, 11, 15, 19, or 25, or an amino acid sequence at least 85% identical thereto; or
(b) a light chain variable region sequence having an amino acid sequence of SEQ ID NOs: 5, 9, 13, 17, 21, 23, or 27 or an amino acid sequence at least 85% identical thereto; or
(c) a heavy chain variable region encoded by a nucleic acid sequence of SEQ ID NOs: 2, 6, 10, 14, 18, or 24, or a nucleic acid sequence at least 85% identical thereto; or
(d) a light chain variable region encoded by a nucleic acid sequence of SEQ ID NOs: 4, 8, 12, 16, 20, 22, or 26, or a nucleic acid sequence at least 85% identical thereto.

3.-16. (canceled)

17. The antibody or epitope binding fragment thereof according to claim 1, which is an IgG or comprises an IgG backbone.

18. (canceled)

19. The antibody or epitope binding fragment thereof according to claim 1, wherein the epitope is an epitope of LOX-1 protein in native conformation, optionally wherein the antibody or epitope binding fragment thereof does not specifically bind to an epitope of LOX-1 protein in denatured condition.

20. The antibody or epitope binding fragment thereof according to claim 1, wherein the epitope is an epitope of LOX-1 protein in denatured condition.

21. The antibody or epitope binding fragment thereof according to claim 1, wherein said antibody is a chimeric antibody, a humanized antibody, a human antibody, a CDR-grafted antibody, a multi-specific binding construct that can bind two or more targets, a dual specific antibody, a bi-specific antibody, an affinity matured antibody, a single-domain antibody (sdAb), a single antibody chain, an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)₂ construct, a monovalent or bivalent construct from which domains non-essential to monoclonal antibody function have been removed, a single-chain molecule containing one light chain variable region (VL), one heavy chain variable region (VH) antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, or a monobody.

22. The antibody or epitope binding fragment thereof according to any claim 1 which is coupled covalently or non-covalently to a detectable label, optionally the detectable label is selected from an enzyme, a fluorescent label, a radioisotope, or a chemiluminescent label.

23. The antibody or epitope biding fragment thereof according to claim 1, which is coupled covalently or non-covalently to a chemotherapy drug or to a radioactive particle.

24. The antibody or epitope binding fragment thereof according to claim 1, which is bi-specific and further comprises an epitope binding fragment which specifically binds to a cell surface biomarker of PMN-MDSC other than LOX-1.

25. (canceled)

26. A diagnostic composition comprising one, two, three, four, or more of the antibody or epitope binding fragment thereof according to claim 1.

27. The composition according to claim 26, wherein the antibody or epitope binding fragment thereof is immobilized on a substrate, optionally the substrate is a plate, an enzyme linked immunosorbent assay (ELISA) plate, a slide, a pipette, a bead, a magnetic bead, a chip, or a microchip.

28. (canceled)

29. A method of diagnosing a cancer or monitoring progression or metastasis of a cancer in a subject comprising:
(a) obtaining a biological sample from the subject;
(b) contacting the sample with the diagnostic composition according to claim 26;
(c) detecting and optionally distinguishing LOX-1 positive cells from LOX-1 negative cells in the sample, and
(d) diagnosing the subject with cancer or cancer metastasis if number of the LOX-1 positive cells is above that of a negative control, or determining cancer progression (for example, tumor size) in the subject by counting the LOX-1 positive cells and comparing it to a control.

30. The method according to claim 29, wherein the contacting step (b) further comprises contacting the sample with a ligand that specifically binds to or forms a complex with a neutrophil biomarker to identify neutrophil or polymorphonuclear neutrophils (PMNs), optionally wherein the neutrophil biomarker is CD15 or CD66b.

31.-32. (canceled)

33. The method according to claim 29, wherein step (c) comprises detecting and optionally distinguishing LOX-1 positive PMNs from LOX-1 negative cells in the sample, and wherein step (d) comprises diagnosing the subject with cancer or cancer metastasis if number of the LOX-1 positive PMNs is above that of a negative control, or determining cancer progression (for example, tumor size) in the subject by counting the LOX-1 positive PMNs and comparing it to a control.

34. (canceled)

35. The method according to claim 29, wherein step (c) comprises washing to reduce or eliminate the LOX-1 negative cells and other debris in the sample.

36. (canceled)

37. The method according to claim 29, further comprising a step of identifying cells with biomarkers shared by both PMN-MDSCs and PMNs, and isolating cells of both PMN-MDSCs and PMNs prior to the contacting step of (b).

38. The method according to claim 29, wherein the biological sample is a fluid sample or a tumor tissue, optionally the biological sample is whole blood and wherein the method further comprises destroying or lysing any red blood cells in the sample.

39. The method according to claim 29, further comprising collecting LOX-1 negative cells which contains PMNs and being substantially free from PMN-MDSCs.

40. The method according to claim 29, wherein a population of LOX-1 positive cells^{cells} greater than 1% of the total neutrophil population in the sample of a subject indicates presence, progression or metastasis of a cancer.

41-45. (canceled)

46. A method of treating a cancer in a subject comprising:
(a) obtaining a biological sample from a subject;
(b) contacting the sample with one or more of the antibody or epitope binding fragment thereof according to claim 1;
(c) detecting whether LOX-1 positive cells (for example, PMN-MDSCs) are present in the sample; and
(d) when presence of LOX-1 is detected, administering an effective amount of a composition that reduces or inhibits ER stress response in mammalian neutrophils or reduces or inhibits LOX-1 expression on neutrophil populations, or administering an effective amount of the antibody or epitope binding fragment thereof according to claim 1.

47.-51. (canceled)


SEQUENCE LISTING (28 sequences)

SEQ ID NO: 1 - protein, 290 aa, Artificial Sequence
  recombinant hLOX-1 protein.
  SIGNAL (1)..(18): Signal peptide
  MISC_FEATURE (19)..(290): human LOX-1 protein
MDWTWILFLVAAATRVHSTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 2 - DNA, 396 bp, Artificial Sequence
  Coding sequence for variable region of the 6A10 heavy chain.
  misc_feature (1)..(57): signal sequence
  misc_feature (58)..(147): Framework 1
  misc_feature (148)..(162): CDR1
  misc_feature (163)..(204): Framework 2
  misc_feature (205)..(255): CDR2
  misc_feature (256)..(351): Framework 3
  misc_feature (352)..(363): CDR3
  misc_feature (364)..(396): Framework 4
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctactacatgcactgggtgaagcaaagccatgtaaagagccttgagtggattggacgtattaatccttacaatggtgctactagctacaaccagaatttcaaggacaaggccagcttgactgtagataagtcctccagcacagcctacatggagctccacagcctgacatctgaggactctgcagtctattactgtgcccccgacgtcgcttactggggccaagggactctggtcactgtctctgca

SEQ ID NO: 3 - protein, 132 aa, Artificial Sequence
  Amino acid sequence of variable region of the 6A10 heavy chain.
  SIGNAL (1)..(19): signal seqeunce
  MISC_FEATURE (20)..(49): FR1
  MISC_FEATURE (50)..(54): CDR1
  MISC_FEATURE (55)..(68): FR2
  MISC_FEATURE (69)..(85): CDR2
  MISC_FEATURE (86)..(117): FR3
  MISC_FEATURE (118)..(121): CDR3
  MISC_FEATURE (122)..(132): FR4
MGWSWIFLFLLSGTAGVLSEVQLQQSGPELVKPGASVKISCKASGYSFTGYYMHWVKQSHVKSLEWIGRINPYNGATSYNQNFKDKASLTVDKSSSTAYMELHSLTSEDSAVYYCAPDVAYWGQGTLVTVSA

SEQ ID NO: 4 - DNA, 384 bp, Artificial Sequence
  Coding sequence for variable region of the 6A10 light chain.
  misc_feature (1)..(60): signal sequence
  misc_feature (61)..(129): Framework 1
  misc_feature (130)..(162): CDR1
  misc_feature (163)..(207): Framework 2
  misc_feature (208)..(228): CDR2
  misc_feature (229)..(324): Framework 3
  misc_feature (325)..(354): CDR3
  misc_feature (355)..(384): Framework 4
atgaggacccctgctcagtttcttggaatcttgttgctctggtttccaggtatcaaatgtgacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttcctccgtggacgttcggtggaggcaccaagctggaaatcaaa

SEQ ID NO: 5 - protein, 128 aa, Artificial Sequence
  Amino acid sequence of variable region of the 6A10 light chain.
  SIGNAL (1)..(20): signal seqeunce
  MISC_FEATURE (21)..(43): FR1
  MISC_FEATURE (44)..(54): CDR1
  MISC_FEATURE (55)..(69): FR2
  MISC_FEATURE (70)..(76): CDR2
  MISC_FEATURE (77)..(108): FR3
  MISC_FEATURE (109)..(118): CDR3
  MISC_FEATURE (119)..(128): FR4
MRTPAQFLGILLLWFPGIKCDIKMTQSPSSMYASLGERVTITCKASQDINSYLSWFQQKPGKSPKTLIYRANRLVDGVPSRFSGSGSGQDYSLTISSLEYEDMGIYYCLQYDEFPPWTFGGGTKLEIK

SEQ ID NO: 6 - DNA, 393 bp, Artificial Sequence
  Coding sequence for variable region of the 9E12 heavy chain.
  misc_feature (1)..(57): signal sequence
  misc_feature (58)..(147): FR1
  misc_feature (58)..(147): FR1
  misc_feature (148)..(162): CDR1
  misc_feature (163)..(204): FR2
  misc_feature (205)..(255): CDR2
  misc_feature (256)..(351): FR3
  misc_feature (352)..(360): CDR3
  misc_feature (361)..(393): FR4
atgggatggagctggatctttctctttctcctgtcaggaactgcaggtgtcctctctcaggtccaactgcagcagcctggggctgagctggtgaagcctggggcttcagtgaagatgtcctgcaaggcttctggctacaccttcaccagctactggatgcactgggtgaagcagaggcctggacaaggccttgagtggatcggaacgattgatccttcagatagttatactagctacaatcaaaagttcaagggcaaggccacattgactgtagacacatcctccagcacagcctacatgcagctcagcagcctgacatctgaggactctgcggtctattactgtgaagggtttgactactggggccaaggcaccactctcacagtctcctca

SEQ ID NO: 7 - protein, 131 aa, Artificial Sequence
  Amino acid sequence of variable region of the 9E12 heavy chain.
  MISC_FEATURE (1)..(19): Signal
  MISC_FEATURE (20)..(49): FR1
  MISC_FEATURE (50)..(54): CDR1
  MISC_FEATURE (55)..(68): FR2
  MISC_FEATURE (69)..(85): CDR2
  MISC_FEATURE (86)..(117): FR3
  MISC_FEATURE (118)..(120): CDR3
  MISC_FEATURE (121)..(131): FR4
MRWSCIILFLVATATGVNSQVQLQQPGAELVKPGASVKMSCKASGYTFTSYWMHWVKQRPGQGLEWIGTIDPSDSYTSYNQKFKGKATLTVDTSSSTAYMQLSSLTSEDSAVYYCEGFDYWGQGTTLTVSS

SEQ ID NO: 8 - DNA, 393 bp, Artificial Sequence
  Coding sequence for variable region of the 9E12 light chain.
  misc_feature (1)..(57): Signal
  misc_feature (58)..(126): FR1
  misc_feature (127)..(174): CDR1
  misc_feature (175)..(219): FR2
  misc_feature (220)..(240): CDR2
  misc_feature (241)..(336): FR3
  misc_feature (337)..(363): CDR3
  misc_feature (364)..(393): FR4
atgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccattcacgttcggctcggggacaaagttggaaataaaa

SEQ ID NO: 9 - protein, 131 aa, Artificial Sequence
  Amino acid sequence of variable region of the 9E12 light chain.
  MISC_FEATURE (1)..(19): signal peptide
  MISC_FEATURE (20)..(42): FR1
  MISC_FEATURE (43)..(58): CDR1
  MISC_FEATURE (59)..(73): FR2
  MISC_FEATURE (74)..(80): CDR2
  MISC_FEATURE (81)..(112): FR3
  MISC_FEATURE (113)..(121): CDR3
  MISC_FEATURE (122)..(131): FR4
MSPAQFLFLLVLWIRETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPFTFGSGTKLEIK

SEQ ID NO: 10 - DNA, 411 bp, Artificial Sequence
  Coding sequence for variable region of the 12D9 heavy chain.
  misc_feature (1)..(57): signal
  misc_feature (58)..(147): FR1
  misc_feature (148)..(162): CDR1
  misc_feature (163)..(204): FR2
  misc_feature (205)..(255): CDR3
  misc_feature (256)..(351): FR3
  misc_feature (352)..(378): CDR3
  misc_feature (379)..(411): FR4
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcagactccaggaaagggtttaaagtggatgggctggataaacacctacactggagagccaacatatactgatgacttcaagggacggtttgccttctctttggaaacctctgccagcactgcctctttgcagatcatcaacctcaaaaatgaggacacggctacatatttctgtgcaagggggggcgatttctatgctatggactactggggtcaaggaacctcagtcaccgtctcctca

SEQ ID NO: 11 - protein, 137 aa, Artificial Sequence
  Amino acid sequence of variable region of the 12D9 heavy chain.
  MISC_FEATURE (1)..(19): signal
  MISC_FEATURE (20)..(49): FR1
  MISC_FEATURE (50)..(54): CDR1
  MISC_FEATURE (55)..(68): FR2
  MISC_FEATURE (86)..(117): FR3
  MISC_FEATURE (118)..(126): CDR3
  MISC_FEATURE (127)..(137): FR4
MAWVWTLLFLMAAAQSAQAQIQLVQSGPELKKPGETVKISCKASGYTFTNYGMNWVKQTPGKGLKWMGWINTYTGEPTYTDDFKGRFAFSLETSASTASLQIINLKNEDTATYFCARGGDFYAMDYWGQGTSVTVSS

SEQ ID NO: 12 - DNA, 381 bp, Artificial Sequence
  Coding sequence for variable region of the 12D9 light chain.
  misc_feature (1)..(60): signal
  misc_feature (61)..(129): FR1
  misc_feature (130)..(162): CDR1
  misc_feature (163)..(207): FR2
  misc_feature (208)..(228): CDR2
  misc_feature (229)..(324): FR3
  misc_feature (325)..(351): CDR3
  misc_feature (352)..(381): FR4
atggaatcacagactctggtcttcatatccatactgctctggttatatggtgctgatgggaacatcgtaatgacccaatctcccaaatccatgtccatgtcagtaggagagagggtcaccttgagctgcaaggccagtgagaatgtgggtacttatgtatcctggtatcaacagaaaccagagcagtctcctaaactggtgatattcggggcatccaaccggaacactggggtccccgatcgcttcacaggcagtggatttgcaacagatttcactctgaccatcagcagtgtgcaggctgaagaccttggagattatcactgtggacagagttccaactatccatacacgttcggaggggggaccaagctggaaataaaa

SEQ ID NO: 13 - protein, 127 aa, Artificial Sequence
  Amino acid sequence of variable region of the 12D9 light chain.
  MISC_FEATURE (1)..(20): signal
  MISC_FEATURE (21)..(43): FR1
  MISC_FEATURE (44)..(54): CDR1
  MISC_FEATURE (55)..(69): FR2
  MISC_FEATURE (70)..(76): CDR2
  MISC_FEATURE (77)..(108): FR3
  MISC_FEATURE (109)..(117): CDR3
  MISC_FEATURE (118)..(127): FR4
MESQTLVFISILLWLYGADGNIVMTQSPKSMSMSVGERVTLSCKASENVGTYVSWYQQKPEQSPKLVIFGASNRNTGVPDRFTGSGFATDFTLTISSVQAEDLGDYHCGQSSNYPYTFGGGTKLEIK

SEQ ID NO: 14 - DNA, 408 bp, Artificial Sequence
  Coding sequence for variable region of the 12E4 heavy chain.
  misc_feature (1)..(57): Signal
  misc_feature (58)..(147): FR1
  misc_feature (148)..(162): CDR1
  misc_feature (163)..(204): FR2
  misc_feature (205)..(255): CDR2
  misc_feature (256)..(351): FR3
  misc_feature (352)..(375): CDR3
  misc_feature (376)..(408): FR4
atgggatggagctggatctttctcttcctcctgtcaggaactgcaggtgtccactctgaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactagctacaaccagaagttcaagggcaaggccacattaactgtagacaagtcatccagcacagcctacatggagctcctcagtctgacatctgaggactctgcagtctattactgtgcaagagggggcttctatgctatggactactggggtcaaggaacctcagtcaccgtctcctca

SEQ ID NO: 15 - protein, 136 aa, Artificial Sequence
  Amino acid sequence of variable region of the 12E4 heavy chain.
  MISC_FEATURE (1)..(19): signal
  MISC_FEATURE (20)..(49): FR1
  MISC_FEATURE (50)..(54): CDR1
  MISC_FEATURE (55)..(68): FR2
  MISC_FEATURE (69)..(85): CDR2
  MISC_FEATURE (86)..(117): FR3
  MISC_FEATURE (118)..(125): CDR3
  MISC_FEATURE (126)..(136): FR4
MGWSWIFLFLLSGTAGVHSEVQLQQSGPELVKPGASMKISCKASGYSFTGYTMNWVKQSHGKNLEWIGLINPYNGGTSYNQKFKGKATLTVDKSSSTAYMELLSLTSEDSAVYYCARGGFYAMDYWGQGTSVTVSS

SEQ ID NO: 16 - DNA, 378 bp, Artificial Sequence
  Coding sequence for variable region of the 12E4 light chain.
  misc_feature (1)..(57): signal
  misc_feature (58)..(126): FR1
  misc_feature (127)..(159): CDR1
  misc_feature (160)..(204): FR2
  misc_feature (205)..(225): CDR2
  misc_feature (226)..(321): FR3
  misc_feature (322)..(348): CDR3
  misc_feature (349)..(378): FR4
atgtcctctgctcagttccttggtctcctgttgctctgttttcaaggtaccagatgtgatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtacacgttcggaggggggaccaagctggaaataaaa

SEQ ID NO: 17 - protein, 126 aa, Artificial Sequence
  Amino acid sequence of variable region of the 12E4 light chain.
  MISC_FEATURE (1)..(19): signal
  MISC_FEATURE (20)..(42): FR1
  MISC_FEATURE (43)..(53): CDR1
  MISC_FEATURE (54)..(68): FR2
  MISC_FEATURE (69)..(75): CDR2
  MISC_FEATURE (76)..(107): FR3
  MISC_FEATURE (108)..(116): CDR3
  MISC_FEATURE (117)..(126): FR4
MSSAQFLGLLLLCFQGTRCDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIK

SEQ ID NO: 18 - DNA, 408 bp, Artificial Sequence
  Coding sequence for variable region of the 4D6 heavy chain.
  misc_feature (1)..(57): signal
  misc_feature (58)..(147): FR1
  misc_feature (148)..(162): CDR1
  misc_feature (168)..(204): FR2
  misc_feature (205)..(255): CDR2
  misc_feature (256)..(351): FR3
  misc_feature (352)..(375): CDR3
  misc_feature (376)..(408): FR4
atggaatggacctgggtctttctcttcctcctgtcagtaactgcaggtgtccgctcccaggttcagctgcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtgatagtacttactacaatgagaacttcaagggcaaggccacattcactgcagatacatcctccaacacagcctacatacaactcagcagcctgacatctgaggactctgccgtctattactgtgcaagtgacagctcgggcttgtttgcttactggggccaagggactctggtcactgtctctgca

SEQ ID NO: 19 - protein, 136 aa, Artificial Sequence
  Amino acid sequence of variable region of the 4D6 heavy chain.
  MISC_FEATURE (1)..(19): signal
  MISC_FEATURE (20)..(49): FR1
  MISC_FEATURE (50)..(54): CDR1
  MISC_FEATURE (55)..(68): FR2
  MISC_FEATURE (69)..(85): CDR2
  MISC_FEATURE (86)..(117): FR3
  MISC_FEATURE (118)..(125): CDR3
  MISC_FEATURE (126)..(136): FR4
MEWTWVFLFLLSVTAGVRSQVQLQQSGAELMKPGASVKISCKATGYTFSSYWIEWVKQRPGHGLEWIGEILPGSDSTYYNENFKGKATFTADTSSNTAYIQLSSLTSEDSAVYYCASDSSGLFAYWGQGTLVTVSA

SEQ ID NO: 20 - DNA, 381 bp, Artificial Sequence
  Coding sequence for variable region of the 4D6 light chain, clone 1.
  misc_feature (1)..(60): Signal
  misc_feature (61)..(129): FR1
  misc_feature (130)..(162): CDR1
  misc_feature (163)..(207): FR2
  misc_feature (208)..(228): CDR2
  misc_feature (229)..(324): FR3
  misc_feature (325)..(351): CDR3
  misc_feature (352)..(381): FR4
atgaagtcacagacccaggtcttcgtatttctactgctctgtgtgtctggtgctcatgggagtattgtgatgacccagactcccaaattcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctccgctcacgttcggtgctgggaccaagctggagctgaaa

SEQ ID NO: 21 - protein, 127 aa, Artificial Sequence
  Amino acid sequence of variable region of the 4D6 light chain, clone 1.
  MISC_FEATURE (1)..(20): Signal
  MISC_FEATURE (21)..(43): FR1
  MISC_FEATURE (44)..(54): CDR1
  MISC_FEATURE (55)..(69): FR2
  MISC_FEATURE (70)..(76): CDR2
  MISC_FEATURE (77)..(108): FR3
  MISC_FEATURE (109)..(117): CDR3
  MISC_FEATURE (118)..(127): FR4
MKSQTQVFVFLLLCVSGAHGSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGAGTKLELK

SEQ ID NO: 22 - DNA, 393 bp, Artificial Sequence
  Coding sequence for variable region of the 4D6 light chain, clone 2.
  misc_feature (1)..(60): Signal
  misc_feature (61)..(129): FR1
  misc_feature (130)..(174): CDR1
  misc_feature (175)..(219): FR2
  misc_feature (220)..(240): CDR2
  misc_feature (241)..(336): FR3
  misc_feature (337)..(363): CDR3
  misc_feature (364)..(393): FR4
atggagacagacccactcctgctatgggtgctgctgctctgggttccaggctccactggtgacattgtgctgacccaatctccagcttctttggctgtgtctctaggacagagggccaccatatcctgccaagccagcgaaagtgtcagttttgctgatacaagtttaatgcactggtaccaacagaaaccaggacagccacccaaactcctcatctatcgtgcatccaacctagaatctggagtccctgccaggttcactggcagtgggtctgagtcagacttcactctcaccatcgatcctgtggaggaagatgatgctgcaatgtattactgtatgcaaagtatggaagatccattcacgttcggctcggggacaaagttggaaataaaa

SEQ ID NO: 23 - protein, 131 aa, Artificial Sequence
  Amino acid sequence of variable region of the 4D6 light chain, clone 2.
  MISC_FEATURE (1)..(20): Signal
  MISC_FEATURE (21)..(43): FR1
  MISC_FEATURE (44)..(58): CDR1
  MISC_FEATURE (59)..(73): FR2
  MISC_FEATURE (74)..(80): CDR2
  MISC_FEATURE (81)..(112): FR3
  MISC_FEATURE (113)..(121): CDR3
  MISC_FEATURE (122)..(131): FR4
METDPLLLWVLLLWVPGSTGDIVLTQSPASLAVSLGQRATISCQASESVSFADTSLMHWYQQKPGQPPKLLIYRASNLESGVPARFTGSGSESDFTLTIDPVEEDDAAMYYCMQSMEDPFTFGSGTKLEIK

SEQ ID NO: 24 - DNA, 411 bp, Artificial Sequence
  Coding sequence for variable region of the 3D8 heavy chain.
  misc_feature (1)..(57): signal
  misc_feature (58)..(147): FR1
  misc_feature (148)..(162): CDR1
  misc_feature (163)..(204): FR2
  misc_feature (205)..(255): CDR2
  misc_feature (256)..(351): FR3
  misc_feature (352)..(378): CDR3
  misc_feature (379)..(411): FR4
atggaatggagttggatatttctctttctcctgtcaggaactgcaggtgtccactctgaggtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaaggggggtctatgatggttaccctgactactggggccaaggcaccactctcacagtctcctca

SEQ ID NO: 25 - protein, 137 aa, Artificial Sequence
  Amino acid sequence of variable region of the 3D8 heavy chain.
  MISC_FEATURE (1)..(19): Signal
  MISC_FEATURE (20)..(49): FR1
  MISC_FEATURE (50)..(54): C DR1
  MISC_FEATURE (55)..(68): FR2
  MISC_FEATURE (69)..(85): CDR2
  MISC_FEATURE (86)..(117): FR3
  MISC_FEATURE (118)..(126): CDR3
  MISC_FEATURE (127)..(137): FR4
MEWSWIFLFLLSGTAGVHSEVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGVYDGYPDYWGQGTTLTVSS

SEQ ID NO: 26 - DNA, 381 bp, Artificial Sequence
  Coding sequence for variable region of the 3D8 light chain.
  misc_feature (1)..(60): signal
  misc_feature (61)..(129): FR1
  misc_feature (130)..(162): CDR1
  misc_feature (163)..(207): FR2
  misc_feature (208)..(228): CDR2
  misc_feature (229)..(324): FR3
  misc_feature (325)..(351): CDR3
  misc_feature (352)..(381): FR4
atggtgtccacttctcagctccttggacttttgcttttctggacttcagcctccagatgtgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgtacacgttcggaggggggaccaagctggaaataaaa

SEQ ID NO: 27 - protein, 127 aa, Artificial Sequence
  Amino acid sequence of variable region of the 3D8 light chain.
  MISC_FEATURE (1)..(20): signal
  MISC_FEATURE (21)..(43): FR1
  MISC_FEATURE (44)..(54): CDR1
  MISC_FEATURE (55)..(69): FR2
  MISC_FEATURE (70)..(76): CDR2
  MISC_FEATURE (77)..(108): FR3
  MISC_FEATURE (109)..(117): CDR3
  MISC_FEATURE (118)..(127): FR4
MVSTSQLLGLLLFWTSASRCDIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 28 - DNA, 2214 bp, Artificial Sequence
  a coding sequence for hLOX-1
atggactggacctggagaatcctgttcctggtggcagcagcaaccggaacacacgccgagatccagctgcagcagacaggaccagagctggtgaagcctggagcctctgtgaagatcagctgcaaggcctccggctaccctttcaccgactatatcatggtgtgggtgaagcagtctcacggcaagagcctggagtggatcggcaacatcagcccatactatggcaccacaaactacaatctgaagtttaagggcaaggccaccctgacagtggataagagctcctctaccgcctatatgcagctgaactctctgacaagcgaggactccgccgtgtactattgcgccagatcccccaattgggatggagcatggttcgcacactggggacagggcgccctggtgaccgtgagctccgcctctacaaagggccctagcgtgtttccactggcaccctctagcaagagcacctccggcggcacagccgccctgggctgtctggtgaaggattacttccccgagcctgtgaccgtgtcttggaacagcggcgccctgaccagcggagtgcacacatttcctgccgtgctgcagtcctctggcctgtactccctgagctccgtggtgaccgtgccatctagctccctgggcacccagacatatatctgcaacgtgaatcacaagccttctaatacaaaggtggataagaaggtggagccaaagagctgtgacaagacccacacatgccctccctgtccagcaccagagctggcaggagcaccaagcgtgttcctgtttccacccaagcccaaggacacactgatgatctcccggaccccagaggtgacatgcgtggtggtggacgtgagccacgaggaccccgaggtgaagttcaactggtacgtggatggcgtggaggtgcacaatgccaagaccaagcccagggaggagcagtacaactccacctatagagtggtgtctgtgctgacagtgctgcaccaggactggctgaacggcaaggagtataagtgcaaggtgagcaataaggccctgcctgccccaatcgagaagaccatctccaaggcaaagggacagcctcgggagccacaggtgtacacactgcctccaagccgcgacgagctgaccaagaaccaggtgtccctgacatgtctggtgaagggcttctatcccagcgatatcgccgtggagtgggagtccaatggccagcctgagaacaattacaagaccacaccccctgtgctggactccgatggctctttctttctgtattctaagctgaccgtggataagagccgctggcagcagggcaacgtgttttcttgcagcgtgatgcacgaggccctgcacaatcactacacccagaagtccctgtctctgagccctggcaagaggggaaggaagaggagatccggctctggcgccacaaacttcagcctgctgaagcaggcaggcgacgtggaggagaatccaggacctatggtgctgcagacccaggtgtttatcagcctgctgctgtggatctccggcgcctacggcgatatcgtgctgacccagagcccagcatccctggccgtgtctctgggacagcgggccacaatcagctgtaaggccagccagtccgtggactacgatggcgactcctatatgaactggtttcagcagaagcccggccagccacccaagctgctgatctacgccgcctccaatctggagtctggcatccccgcacgcttctctggcagcggctccggaaccgacttcaccctgaacatccaccctgtggaggaggaggacgcagcaacctactattgccagcagtccaatgaggaccccttcaccttcggcagcggcacaaagctggagatcaagaggaccgtggccgccccctccgtgttcatctttcctccatctgacgagcagctgaagagcggcacagcctccgtggtgtgcctgctgaacaatttctaccctagagaggccaaggtgcagtggaaggtggataacgccctgcagtctggcaatagccaggagtccgtgaccgagcaggattctaaggacagcacatattccctgtctagcaccctgacactgagcaaggccgactacgagaagcacaaggtgtatgcatgcgaggtgacccaccagggactgtcctctccagtgacaaagtcctttaacagaggcgagtgttgataa